(19) United States
(12) Patent Application Publication
(10) Pub. No.: US 2025/0304585 A1
(43) Pub. Date: Oct. 2, 2025

(54) SULFAMATE BASED COVALENT LIGAND-DIRECTED RELEASE (COLDR) COMPOUNDS

(71) Applicant: YEDA RESEARCH AND DEVELOPMENT CO. LTD., Rehovot (IL)

(72) Inventors: Nir LONDON, Rehovot (IL); Rambabu REDDI, Rehovot (IL)

(73) Assignee: YEDA RESEARCH AND DEVELOPMENT CO. LTD., Rehovot (IL)

(21) Appl. No.: 18/864,916

(22) PCT Filed: Jun. 14, 2023
(86) PCT No.: PCT/IL2023/050613

§ 371 Date: Nov. 12, 2024

Related U.S. Application Data
(60) Provisional application No. 63/351,927, filed on Jun. 14, 2022.
(60) Provisional application No. 63/384,955, filed on Nov. 24, 2022.

(51) Int. Cl. C07D 473/00; A61K 31/52; A61P 35/02

(52) U.S. Cl. CPC C07D 473/00; A61K 31/52; A61P 35/02

(57) ABSTRACT

Provided herein electrophiles (I) comprising a sulfamate group for covalent ligand-directed release chemistry (CoLDR). Formula(I).

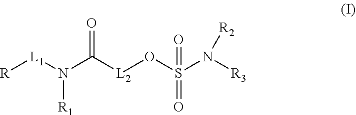

### FIELD OF INTEREST

This invention is directed to electrophiles comprising a sulfamate group for covalent ligand-directed release chemistry (CoLDR).

### BACKGROUND

Electrophilic small molecules that are able to form covalent bonds with nucleophilic amino acids like cysteine, lysine and tyrosine, play a pivotal role in chemical biology. Such electrophiles have been successfully used in bioconjugation for the synthesis of antibody-drug conjugates, used as probes for chemoproteomics activity based protein profiling (ABPP), and as covalent warheads in the design of targeted covalent inhibitors (TCIs).

Highly reactive and residue-selective electrophiles are useful for bioconjugation and proteomics applications, while low reactivity and highly stable electrophiles are suitable for targeted covalent inhibitors (TCIs). Relatively few electrophiles meet the criteria to be used in TCIs. In spite of the therapeutic benefits of covalent inhibitors like enhanced and sustained pharmacological potency and protein isoform selectivity compared to their reversible counterparts, their potential toxicity due to the off-target reactivity is a key concern. Some of the most commonly used electrophiles in designing targeted covalent inhibitors are acrylamides and chloroacetamides which react with cysteines. While acrylamide-based electrophiles are known to be able to achieve sufficiently low reactivity, chloroacetamides are more reactive¹⁻⁴ as covalent ‘warheads’. This greatly limits their application in designing targeted covalent inhibitors (TCIs). Consequently, fluorochloro-acetamide⁵, α-substituted chloroacetamides^{6,7} and di- and tri halo acetamide⁸ warheads have been reported as less reactive alternatives (FIG. 1A). Although these warheads showed improved selectivity, it was typically at the cost of reduced potency. Tunability of the electrophile reactivity can help to find the optimal balance between selectivity and potency. However, there are very few degrees of freedom with chloroacetamides.

Several strategies were reported for the functionalization of covalent binders beyond just enzyme inhibition. In this context, covalent inhibitor-based fluorescent turn-on probes have been developed and used in protein profiling and sensing applicaitons⁹⁻¹¹. Tamura et al¹² recently developed N-acyl-N-alkyl sulfonamide (NASA) electrophiles which has been used for site-selective labeling of a protein of interest (POI) while eliminating the recognition element¹². These chemistries end up covalently bound to the POI but do not release ‘payloads’ as a result. Applicant has previously developed substituted methacrylamides as an electrophilic warhead which enabled covalent ligand directed release (CoLDR). Using this chemistry, fluorescent or chemiluminescent payloads were released in their active form upon reacting with the target cysteine¹³. Substituted methacrylamides also allowed the site-specific labeling of proteins in their active form¹⁴. However, this chemistry is limited to acrylamide-based covalent inhibitors.

Sulfamates (—O—SO₂—NR—; R=H, alkyl, aryl) are prevalent in medicinal chemistry and many bioactive and drug molecules contain this functionality.

Provided herein α-sulfamate acetamides as highly stable warheads with tunable reactivity and similar geometry to chloroacetamides. (FIG. 1A, right).

The sulfamates provided herein, expand the CoLDR chemistry concept to analogs of α-halo acetamide electrophiles.

### REFERENCES

1. Flanagan, M. E. et al. Chemical and computational methods for the characterization of covalent reactive groups for the prospective design of irreversible inhibitors. J. Med Chem. 57, 10072-10079 (2014).
2. Resnick, E. et al. Rapid Covalent-Probe Discovery by Electrophile-Fragment Screening. J. Am. Chem. Soc. 141, 8951-8968 (2019).
3. Gabizon, R., Resnick, E. & London, N. CHAPTER 4: Best Practices for Design and Characterization of Covalent Chemical Probes. in The Discovery and Utility of Chemical Probes in Target Discovery 69-99 (2020).
4. Henning, N. J. et al. Discovery of a Covalent FEM1B Recruiter for Targeted Protein Degradation Applications. J Am. Chem. Soc. 144, 701-708 (2022).
5. Shindo, N. et al. Selective and reversible modification of kinase cysteines with chlorofluoroacetamides. Nat. Chem. Biol. 15, 250-258 (2019).
6. Gehringer, M. & Laufer, S. A. Emerging and Re-Emerging Warheads for Targeted Covalent Inhibitors: Applications in Medicinal Chemistry and Chemical Biology. J. Med. Chem. 62, 5673-5724 (2019).
7. Allimuthu, D. & Adams, D. J. 2-Chloropropionamide As a Low-Reactivity Electrophile for Irreversible Small-Molecule Probe Identification. ACS Chem. Biol. 12, 2124-2131 (2017).
8. Ma, C. et al. Discovery of Di- and Trihaloacetamides as Covalent SARS-CoV-2 Main Protease Inhibitors with High Target Specificity. J. Am. Chem. Soc. 143, 20697-20709 (2021).
9. Kojima, H. et al. Cyclization Reaction-Based Turn-on Probe for Covalent Labeling of Target Proteins. Cell Chem Biol 27, 334-349.e11 (2020).
10. Chen, J., Wang, X., He, F. & Pan, Z. Development of a Selective Labeling Probe for Bruton's Tyrosine Kinase Quantification in Live Cells. Bioconjugate Chemistry vol. 29 1640-1645 (2018).
11. Wang, X., Ma, N., Wu, R., Ding, K. & Li, Z. A novel reactive turn-on probe capable of selective profiling and no-wash imaging of Bruton's tyrosine kinase in live cells. Chemical Communications 55 3473-3476 (2019).
12. Tamura, T. et al. Rapid labelling and covalent inhibition of intracellular native proteins using ligand-directed N-acyl-N-alkyl sulfonamide. Nature Communications vol. 9 (2018).
13. Reddi, R. N. et al. Tunable Methacrylamides for Covalent Ligand Directed Release Chemistry. Journal of the American Chemical Society vol. 143 4979-4992 (2021).
14. Reddi, R. N. et al. Site-Specific Labeling of Endogenous Proteins Using CoLDR Chemistry. J. Am. Chem. Soc. 143, 20095-20108 (2021).

### SUMMARY

In some embodiments provided herein a Covalent Ligand Directed Releasing (CoLDR) compound or pharmaceutically acceptable salt thereof, wherein the CoLDR compound is represented by the structure of formula I.

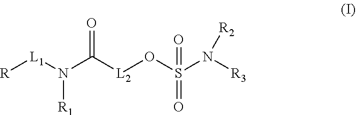

wherein:
R is a protein binding ligand, wherein the nitrogen (NR₁) is linked to the protein binding ligand via L₁ linker or L₁ is a bond and the nitrogen is an atom within the protein binding ligand;
each of R₁ and R₂ are independently H, substituted or unsubstituted: linear or branched alkyl, linear or branched alkenyl, linear or branched alkynyl, aryl, alkylaryl, cycloalkyl, heterocycloalkyl or heteroaryl;
R₃ is H, substituted or unsubstituted: linear or branched alkyl, linear or branched alkenyl, linear or branched alkynyl, aryl, alkyl aryl, cycloalkyl, heterocycloalkyl, heteroaryl a fluorescent probe, a chemiluminescent probe or a radiolabeled probe, a bioactive group; or R₂ and R₃ can form together a five or six membered ring with the nitrogen;
L₁ is a bond, or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group; and
L₂ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group.

In some embodiments provided herein a Covalent Ligand Directed Releasing (CoLDR) compound or pharmaceutically acceptable salt thereof, wherein the CoLDR compound is represented by the structure of formula II:

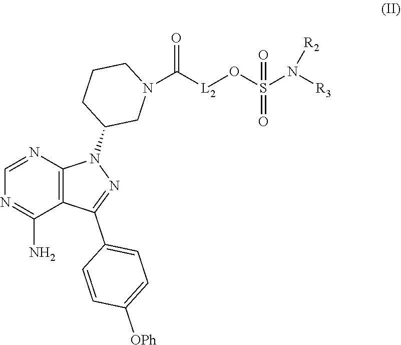

wherein L₂, R₂ and R₃ are as defined for the structure of formula I.

In some embodiments, provided herein a Covalent Ligand Directed Releasing (CoLDR) compound or pharmaceutically acceptable salt thereof wherein the CoLDR compound is represented represented by the structure of formula III:

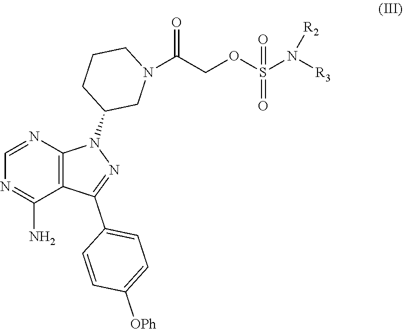

wherein R₂ and R₃ are as defined for the structure of formula I.

In some embodiments provided herein a pharmaceutical composition comprising the Covalent Ligand Directed Releasing (CoLDR) compound of formula I, II or III and a pharmaceutical acceptable carrier.

In some embodiments, provided herein a protein sensor or a protein label comprising a Covalent Ligand Directed Releasing (CoLDR) Compound of formula I, II or III, wherein R₃ is a fluorescent probe or a chemiluminescent probe, wherein, upon interaction between a protein and the protein binding ligand, the fluorescent probe or the chemiluminescent probe is released and the protein binding ligand is covalently attached to the protein and thereby results in change in fluorescence or chemiluminescence of the probes.

### BRIEF DESCRIPTION OF THE DRAWINGS

The subject matter regarded as the analog compounds and uses thereof is particularly pointed out and distinctly claimed in the concluding portion of the specification. The synthetic analog compounds and uses thereof, however, both as to organization and method of operation, together with objects, features, and advantages thereof, may best be understood by reference to the following detailed description when read with the accompanying drawings in which:

FIGS. 1A and 1B present Sulfamate acetamides as electrophiles for targeted covalent inhibitors and CoLDR chemistry: FIG. 1A: Reactivity pattern of α-substituted acetamides. FIG. 1B: Schematic representation of the reaction of a target cysteine with α-sulfamate acetamides through CoLDR chemistry.

FIGS. 2A-2E: present Ibrutinib sulfamates as potent BTK inhibitors: FIG. 2A: Chemical structures of Ibrutinib, 3a-3g. FIG. 2B: Deconvoluted LC/MS spectrum of BTK (2 μM) incubated with 3c (2 μM) at pH 8, 25° C., 30 min. The adduct mass corresponds to a labeling event in which methyl sulfamoic acid was released, validating the proposed mechanism. FIG. 2C: % of labeling of BTK (2 μM) with the probes (3a-3e; 2 μM) at 10 min and 30 min in 20 mM Tris buffer at pH 8, 25° C. FIG. 2D: In vitro kinase activity assay (0.5 nM BTK, 5 μM ATP) for 3a-3g (IC50 of 3a=0.0294 μM, 3b=0.0464 μM, 3c=0.00864 μM, 3d=0.0242 μM, 3e=0.0949 μM, 3g=0.582 μM). FIG. 2E: The correlation of GSH half-life (t_{1/2}) of Ibrutinib sulfamates with measured IC₅₀s in a kinase inhibition assay.

FIGS. 3A-3B present GSH reactivity of Ibrutinib derivatives: FIG. 3A: Rates of depletion of compounds (3a-3e) in a reaction between 10 μM compound and 5 mM GSH in PBS buffer at pH 8, 14° C. (n=2) for 6 h. FIG. 3B: Extrapolated GSH half-lives (t_{1/2}) of the compounds.

FIGS. 4A-4C present Metabolic stability assay for Ibrutinib sulfamates. FIG. 4A: Time-dependent metabolic stability of Ibrutinib, 3a, 3c, and 3d. FIG. 4B, FIG. 4C: Zoom-in of the 5 min and 15 min time points, respectively (n=2).

FIGS. 5A-5G present Ibrutinib sulfamate acetamide analogs are highly potent in cells and in vivo. FIG. 5A: Dose-dependent BTK activity assay in Mino cells as measured by autophosphorylation of BTK. The cells were incubated for 2 h with either 0.1% DMSO, various concentrations of Ibrutinib, or 3a-3d. The cells were activated with anti-IgM and BTK autophosphorylation was quantified by western blot and normalized with respect to β-actin. IC₅₀s were calculated by fitting the data to a dose-response curve using the Prism software. FIG. 5B: Dose-dependent inhibition of B-cell response (as measured by CD86 expression) after anti-IgM-induced activation and treatment with Ibrutinib analogs (3a-3d) for 24 h (n=3; error bars indicate standard deviation). FIG. 5C: Dose-dependent inhibition of pBTK and its downstream pathways (pPLCγ2, pAkt, and pERK) by Ibrutinib derivatives (3a, 3c, 3d & 3e) in CLL patient samples. CLL cells (20×10⁶/mL) were incubated with Ibrutinib or Ibrutinib-based compounds at the indicated doses at 37° C. DMSO treated cells served as controls. After 2 hours of incubation, the cells were either stimulated with goat F(ab′)2 anti-human IgM (10 μg/mL) for 15 minutes or left untreated. Proteins were then extracted and subjected to Western blot analysis. FIG. 5D: Schematic representation of the in vivo mice experiment. Cells isolated from old TCL1 mice spleens, with a malignant cell population higher than 60%, were injected into the tail vein of 6-weeks-old recipient mice. The mice were given a solution containing sulfamate 3c (0.16 mg/mL in 1% cyclodextrin water) ad libitum in the drinking water. Progression of the disease was followed in the peripheral blood (PB) by using flow cytometry for quantification of the IgM+/CD5+ population. FIG. 5E: The IgM+/CD5+ cell population is significantly lowered in 3c treated mice (n=5) compared to untreated (n=3) **p=0.002 for days 7^{th} and 15^{th} (single tailed student's T-test) FIG. 5F: BTK engagement of compound 3c in vivo. Dissected spleens were extracted with RIPA buffer and incubated with an Ibrutinib alkyne analog (‘probe-4’) [Lanning, B. R. et al. A Road Map to Evaluate the Proteome-Wide Selectivity of Covalent Kinase Inhibitors. Nat. Chem. Biol. 2014, 10 (9), 760-767 which is incorporated herein by reference] for 1 h followed by CuAAC reaction with TAMRA-azide in lysate before imaging. FIG. 5G: IsoDTB ABPP experiment with Ibrutinib and sulfamate 3c. Mino cells were treated with 1 μM of either Ibrutinib, or 3c for 2 h followed by incubation of Iodo-acetamide alkyne and CuAAC click reaction with heavy/light isoDTB tags. The labeled peptides were pulled-down with streptavidin beads and quantified via LC/MS/MS (n=4). Proteins in the box have a heavy to light (H/L) ratio≥3. Only peptides detected in at least three out of four repetitions are presented. FIG. 5H. Selectivity of Ibrutinib, 3c, and 3d quantified via a competitive pull-down proteomics experiment. Mino cells are treated with 1 μM compound for 1 h and 10 μM Ibrutinib-alkyne for an additional 1 h (n=4). Proteins were quantified using label-free quantification. Proteins in the box show a significant change (Fold change>2; p<0.05).

FIG. 6 presents dose-dependent inhibition of B cell response (as measured by % CD86 expression) after anti-IgM-induced activation and treatment with Ibrutinib analogs (3a-3g) for 24 h (n=3; error bars indicate standard deviation).

FIGS. 7A-7H present sulfamate chemistry based Covalent Ligand Directed Release (CoLDR) probes. FIG. 7A: Chemical structure of Ibrutinib-based “turn-on” releasing probe (3 h). FIG. 7B: Time dependent increase of fluorescence intensity (representing the release of the coumarin moiety) measured at Ex/Em=385/435 nm (n=3). The compound in and of itself (2 μM) is not fluorescent. Upon mixing of probe and target (2 μM), we see an increase in fluorescence. Pre-incubation of the protein with Ibrutinib prevents the fluorescence. FIG. 7C: Deconvoluted LC/MS spectra for BTK incubated with 3 h at the end of the fluorescence measurement. The adduct mass corresponds to a labeling event in which the coumarin sulfamate (indicated as X) moiety was released, validating the proposed mechanism. FIG. 7D: Schematic representation of the tagging of proteins with release of ligand. The target cysteine reaction at electrophilic sulfamates center followed by the concomitant release of ligand through CoLDR chemistry. FIG. 7E: Chemical structures of Ibrutinib-directed sulfamates with methyl and alkyne tag. FIG. 7F: Deconvoluted LC/MS spectrum shows the labeling of alkyne probe (3j) and demonstrates Ibr-H leaving. FIG. 7G: Cellular labeling profile of 3j (100 nM) after 2 h of incubation with Mino cells. The samples were further reacted with TAMRA-azide in lysate before imaging. An arrow indicates BTK's MW. Upon competition with Ibrutinib (preincubated for 30 min; 1 μM) BTK labeling by 3j is lost. FIG. 7H: BTK activity assay: Mino cells were incubated for 2 h with either DMSO or 1 μM 3j, and then incubated for 45 min with Ibrutinib (100 nM). The cells were washed before induction of BTK activity by anti-IgM. The CoLDR probe was able to rescue BTK activity from inhibition by Ibrutinib.

FIGS. 8A-8B present a CoLDR sulfamate probe. FIG. 8A: Synthesis of CoLDR probe 3 h. FIG. 8B: LC/MS trace of the reaction of compound (100 μM) with 5 mM GSH at pH 8, 37° C. 4-nitrocyano benzene has been used as a reference. The spectrum shows the formation of GSH adduct with the release of coumarin moiety.

FIGS. 9A-9B present labeling and GSH reactivity assay of CoLDR probes. FIG. 9A: Deconvoluted MS spectra (intact protein LC/MS) of 2 μM BTK incubated with 200 μM 3i at pH 8.0, 25° C., 40 min. FIG. 9B: UV spectra (220-400 nm) of the LC/MS analysis of 5 mM GSH incubated with 100 μM of compounds 3j at the last measured point of the GSH t_{1/2} experiment, similar to FIGS. 3A-3B.

It will be appreciated that for simplicity and clarity of illustration, elements shown in the figures have not necessarily been drawn to scale. Further, where considered appropriate, reference numerals may be repeated among the figures to indicate corresponding or analogous elements.

### DETAILED DESCRIPTION

This invention is directed to sulfamate acetamide compounds as electrophilic warheads with varied reactivity, in the context of targeted covalent inhibitors.

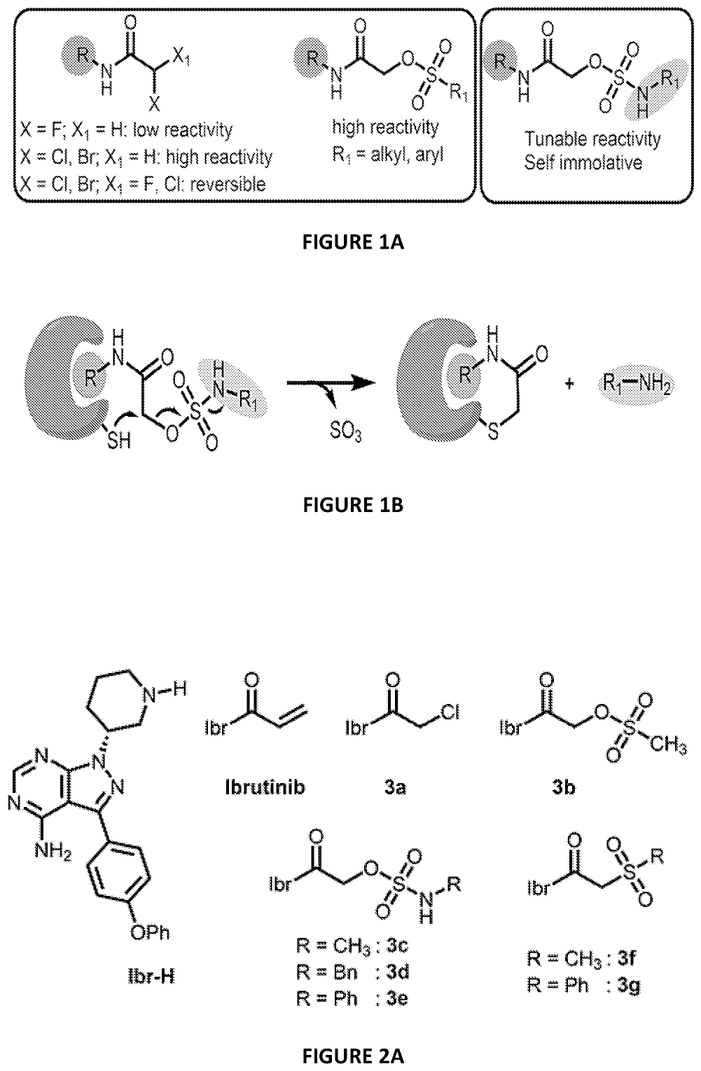

Sulfamate compounds can have varied reactivity based on the nature of the amine group and can act as electrophilic warheads. Further, when these electrophiles react with a nucleophile (such as thiol, an amine or a hydroxyl group), they release sulfamic acid which will dissociate into sulfur trioxide and a free amine (FIG. 1i). This ‘self immolative’ property position them for use in covalent ligand-directed release chemistry.

Provided herein sulfamate acetamide as an electrophilic warhead with varied reactivity, specifically in the context of covalent inhibitors of BTK (Ibrutinib). Since they release an amine functional group after the formation of a covalent bond with a target cysteine, the amine can be used as a ‘payload’ such as a fluorescent turn-on probe for BTK. In addition, the sulfamates compounds provided herein can be used for ligand-directed site-specific traceless labeling of BTK in its active form.

In some embodiments, provided herein a compound or a pharmaceutically acceptable salt thereof, wherein the compound is represented by the structure of formula IA:

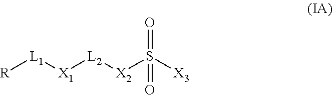

wherein:
X₁ is a bond, N(R₁), N(R₃), O, CH₂, N(R₁)C(O), amide, ketone (C═O), ester, urea or thiourea;
X₂ is a bond, N(R₂), N(R₃) or O;
X₃ is N(R₂)(R₃), O-alkyl, O-alkenyl, O-alkynyl or O—CH₂CCH.
R is a protein binding ligand;
each of R₁ and R₂ are independently H, substituted or unsubstituted: linear or branched alkyl, linear or branched alkenyl, linear or branched alkynyl, aryl, alkyl aryl, cycloalkyl, heterocycloalkyl or heteroaryl;
R₃ is absent, H, substituted or unsubstituted: linear or branched alkyl, linear or branched alkenyl, linear or branched alkynyl, aryl, alkyl aryl, cycloalkyl, heterocycloalkyl, heteroaryl a fluorescent probe, a chemiluminescent probe, a radiolabeled probe or a bioactive group; or R₂ and R₃ can form together a five or six membered ring with the nitrogen;
L₁ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group; and
L₂ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group.

In some embodiments provided herein a compound or pharmaceutically acceptable salt thereof, wherein the compound is represented by the structure of formula I:

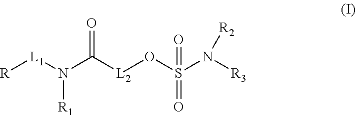

wherein:
R is a protein binding ligand, wherein the nitrogen (NR₁) is linked to the protein binding ligand via L₁ linker or L₁ is a bond and the nitrogen is an atom within the protein binding ligand;
each of R₁ and R₂ are independently H, substituted or unsubstituted: linear or branched alkyl, linear or branched alkenyl, linear or branched alkynyl, aryl, alkyl aryl, cycloalkyl, heterocycloalkyl or heteroaryl;
R₃ is H, substituted or unsubstituted: linear or branched alkyl, linear or branched alkenyl, linear or branched alkynyl, aryl, alkyl aryl, cycloalkyl, heterocycloalkyl, heteroaryl a fluorescent probe, a chemiluminescent probe or a radiolabeled probe or a bioactive group; or R₂ and R₃ can form together a five or six membered ring with the nitrogen;
L₁ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group; and
L₂ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group.

In some embodiments provided herein a compound or pharmaceutically acceptable salt thereof, wherein the compound is represented by the structure of formula IIA:

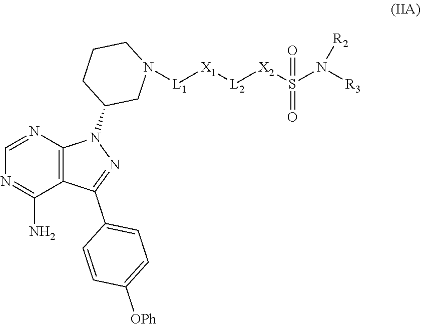

wherein:
X₁ is a bond, N(R₁), N(R₃), O, CH₂, N(R₁)C(O), amide, ketone (C═O), ester, urea or thiourea;
X₂ is a bond, N(R₂), NR₃ or 0;
each of R₁ and R₂ are independently H, substituted or unsubstituted: linear or branched alkyl, linear or branched alkenyl, linear or branched alkynyl, aryl, alkyl aryl, cycloalkyl, heterocycloalkyl or heteroaryl;
R₃ is absent, H, substituted or unsubstituted: linear or branched alkyl, linear or branched alkenyl, linear or branched alkynyl, aryl, alkyl aryl, cycloalkyl, heterocycloalkyl, heteroaryl a fluorescent probe, a chemiluminescent probe or a radiolabeled probe or a bioactive group; or R₂ and R₃ can form together a five or six membered ring with the nitrogen;
L₁ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group; and
L₂ is a bond or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group.

In some embodiments provided herein a compound or pharmaceutically acceptable salt thereof, wherein the compound is represented by the structure of formula II:

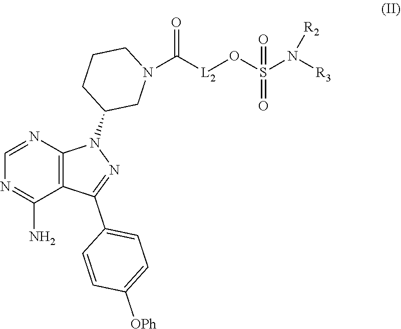

wherein L₂, R₂ and R₃ are as defined for the structure of formula I.

In some embodiments provided herein a compound or pharmaceutically acceptable salt thereof, wherein the compound is represented by the structure of formula III:

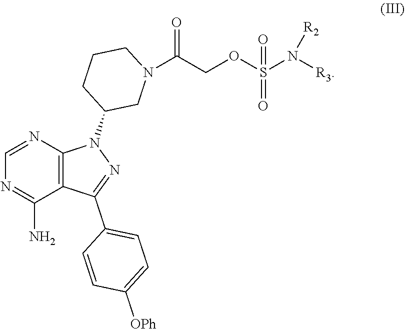

wherein R₂ and R₃ are as defined for the structure of formula I.

In some embodiments provided herein a compound or pharmaceutically acceptable salt thereof, wherein the compound is by the following structures:

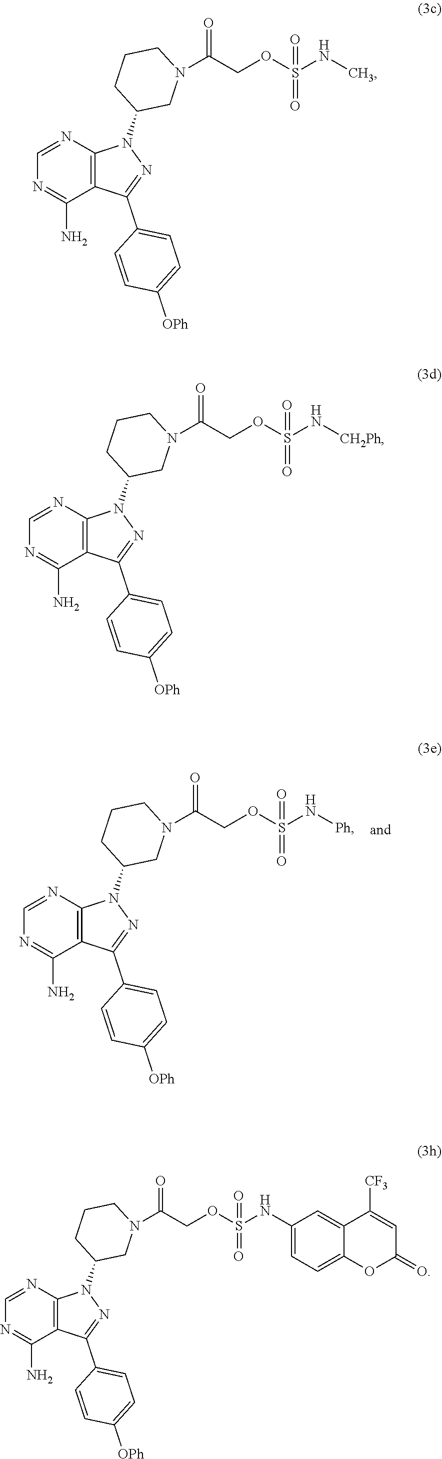

In some embodiments, provided herein a compound of formula I, IA, II, IIA or III wherein the compound is a Covalent Ligand Directed Releasing (CoLDR) Compound.

In some embodiments, provided herein a compound of formula 3c, 3d, 3e, 3 h wherein the compound is a Covalent Ligand Directed Releasing (CoLDR) Compound.

In some embodiments, provided herein a Covalent Ligand Directed Releasing (CoLDR) Compound of formula I, IA, II, IIA or III, wherein upon interaction between a protein and the protein binding ligand (such as the ibrutinib group), SO₃N(R₂)(R₃) is released (FIG. 1). In another embodiment, wherein upon interaction between a protein and the protein binding ligand, a functionalized amine [NH(R₂)(R₃)] is released and sulfur trioxide amine functionality with the release of sulfur trioxide (FIG. 1).

In some embodiments, provided herein a compound of formula I, IA, II, IIA or III, wherein a covalent bond is formed between a protein and the protein binding ligand. In some embodiments, provided herein a compound of formula I, IA, II, HA or III, wherein a covalent bond is formed between a protein and the protein binding ligand as targeted covalent inhibitors. In some embodiments, provided herein a Covalent Ligand Directed Releasing (CoLDR) compound of formula I, IA, II, IIA or III, wherein a covalent bond is formed between a protein and the protein binding ligand. In other embodiments, the covalent bond is formed via a nucleophilic moiety of the protein being a thiol, an amine or a hydroxyl group and the alpha sulfamate acetamide of the compounds of formula I, IA, II, IIA or III.

In some embodiments, provided herein a pharmaceutical composition comprising the compounds of this invention. In some embodiments, provided herein a pharmaceutical composition comprising a compound of formula I, IA, II, IIA or III and a pharmaceutically acceptable carrier.

In some embodiments, provided herein a protein sensor or a protein label comprising a compound of formula I, IA, II, IIA or III, wherein R₃ is a fluorescent probe or a chemiluminescent probe. In some embodiments, provided herein a protein sensor or a protein label comprising a Covalent Ligand Directed Releasing (CoLDR) Compound of formula I, IA, II, IIA or III, wherein R₃ is a fluorescent probe or a chemiluminescent probe, wherein, upon interaction between a protein and the protein binding ligand, the fluorescent probe or the chemiluminescent probe is released and the protein binding ligand is covalently attached to the protein and thereby results in change in fluorescence or chemiluminescence of the probes. In other embodiments, a covalent bond is formed between the protein and the protein binding ligand. In other embodiments, the covalent bond is formed via a nucleophilic group of the protein being a thiol, an amine or a hydroxyl group and the alpha sulfamate acetamide of the CoLDR Compound of formula I, IA, II, IIA or III.

In some embodiments, the sulfamate acetamide compounds provided herein are Covalent Ligand Directed Releasing (CoLDR) Compounds possessing (1) a protein binding ligand and (2) a sulfamate substituted by a fluorescent probe, a chemiluminescent probe or a radiolabeled probe, a bioactive group; wherein the protein binding ligand is covalently linked to a protein and the fluorescent, the chemiluminescent or the radiolabeled probe or any bioactive group is released, upon binding to the protein.

The Covalent Ligand Directed Releasing (CoLDR) Compounds of this invention can be used to modulate the reactivity of selective covalent inhibitors, sensors, diagnostics or can be used as turn-on probes against proteins.

In some embodiments, the compounds of formula I, IA, II, IIA or III of this invention comprise: (1) a protein binding ligand (R or ibrutinib group) and (2) a fluorescent, a chemiluminescent, a radiolabeled probe, a hydrophobic tag or a bioactive group (R₃).

In some embodiments, the compound of formula I, IA, II, IIA or III comprises a protein binding ligand (R or ibrutinib group). In another embodiment, the protein binding ligand (R of formula I, IA) comprises afatinib, Ibrutinib, Evobrutinib, AMG-510, Mpro inhibitors, PL pro inhibitor or derivatives thereof. In another embodiment, a non-limiting example of a protein binding ligand is afatinib or poziotinib or osimertinib or neratinib and its targeted protein is EGFR. In another embodiment, a non-limiting example of a protein binding ligand is Ibrutinib or zanubrutinib or evobrutinib or remibrutinib or spebrutinib and its targeted protein is BTK or BLK. In another embodiment, a non-limiting example of a protein binding ligand is AMG-510 or ARS-1620 or MRTX849 and its targeted protein is K-Ras^{G12C}. In another embodiment, a non-limiting example of a protein binding ligand is PF-06651600 and its protein target is JAK3. In another embodiment, a non-limiting example of a protein binding ligand is Futibatinib or FIIN1 or FIIN2 or FIIN3, PRN1371 and its protein target is FGFR. In another embodiment, a non-limiting example of a protein binding ligand is NU6300 and its protein target is CDK2. In another embodiment, a non-limiting example of a protein binding ligand is THZ1 and its protein target is CDK7. In another embodiment, a non-limiting example of a protein binding ligand is THZ531 and its protein target is CDK12 or CDK13. In another embodiment, a non-limiting example of a protein binding ligand is CNX-1351 and its protein target is PI3Kα. In another embodiment, a non-limiting example of a protein binding ligand is JNK-IN-8 (or derivatives or analogs thereof) and its protein target is JNK. In another embodiment, a non-limiting example of a protein binding ligand is MKK7-COV-3 (or derivatives or analogs thereof) and its protein target is MKK7. In another embodiment, a non-limiting example of a protein binding ligand is CC-90003 and its protein target is ERK1 or ERK2. In another embodiment, a non-limiting example of a protein binding ligand is E6201 and its protein target is MEK1. In another embodiments, Mpro inhibitors are presented in Table 2.

In some embodiments, the compounds provided herein of formula I, IA, II, IIA or III comprise a bioactive group (R₃ of formula I, IA, II, IIA or III). In other embodiments, the bioactive group (R₃ of formula I, IA, II, IIA or III) includes, but not limited to an approved drug, a targeted inhibitor, a cytotoxic, a chemotherapeutic, amino acid side chains, a protein binding ligand, a radiopharmaceutical, substructure or derivative thereof or any chemical modification that elicits a biological perturbation. In another embodiments, N(R₂)(R₃) is part of a protein binding ligand.

“Targeted Inhibitor” as referred herein is a small molecule that shows selective binding of a specific protein or specific protein family. Non limiting examples of targeted inhibitor include: AMG-510, CCT251545, A-366, CPI-169, T0901317, BAY-3827, CM11, Veliparib, BI-1935, SD-36, XMD-12, TH5427, AMG232, 25CN-NBOH, GSK2334470, UNC0642, MRK-740, GSK343, BYL-719, MK-5108, RO5353, AX15836, PD0332991, EPZ015666, Luminespib, CPI-360, OICR-9429, PT2399, S63845, Venetoclax, THZ531, CGI1746, (R)-PFI-2, MI-77301, EPZ004777, Linsitinib, Ruxolitinib, FS-694, CPI-0610, CP-724714, GSK481, BTZO-1, MT1, MS023, SCH772984, BAY-1816032, FM-381, Niraparib, UNC1215, SR-318, MRTX849, A-196, CCT251236, JQ1, CH5424802, AT1, BAY-598, UCSF7447, AM-6761, VX-745, PFI-1, PFI-3, GSK4027, SGC0946, SGC707, EED226, BGJ-398, BLU9931, Tofacitinib, GDC-0879, P505-15, PF-CBP1, AMG900, Skepinone-L, AZD2014, GSK484, CHIR-99021, (R)-9s, UCSF4226, NVS-PAK1-1, EI1, KZR-504, AZD1152, SGX-523, CCT241533, RG7388, VH298, PF-477736, BMS-911543, AB680, BAY1125976, GSK583, BI-2545, EPZ-5676, G-5555, A-395, GNF-5, Romidepsin, EPZ011989, ULK-101, THPP-1, D0264, BAY-707, MZ1, UNC1999, WEHI-539, NVP-AEW541, THZ1, AMG-18, JNK-IN-8, BiBET, EPZ-6438, GSK-J4, CCT244747, CPI-1612, KI-696, PF3644022, SGC-CBP30, Tubacin, Selumetinib, Rapamycin, GSK591, ML323, ABBV-744, AC220, Talazoparib, PDD00017273, Filgotinib, A-485, RG7112, BAZ2-ICR, MI-888, BMX-IN-1, BI-9564, PF-3758309, BAY-985, MCC950, UNC2025, AZD-6482, RGFP966, Bistramide A, Ogerin, I-BRD9, I-CBP112, Eleutherobin, GSK864, Salvinorin A, MLi-2, ICI-199441, BIX-02188, Olaparib, A-1155463, WZ4003, KH-CB19, Tubastatin A, AMG 176, eCF309, E7449, AZ191, BAY-826, R02468, ABT-100, XMD8-87, NI-57, NMS-P118, GW3965, eCF506, ACY-738, BAY-549, HG-9-91-01, WM-1119, T-26c, AZ6102, Glyburide, Pevonedistat, GNE7915, Relacatib, Bafetinib, Pictilisib, Afatinib, VE-821, A-1210477, AVL-292, XMD8-92, RUSKI-201, UNC3866, MPSI-IN-1, GNE-2861, ST0609, AZ0108, I-BET151, BAY-885, 2-MT 63, DDR1-IN-1, EPZ020411, CPI-1205, TP-004, Repaglinide, L-Moses, LXR-623, GSK-5959, CPI-637, GPR40ant39, UNC0638, GSK2801, M-808, JAK3i, CX-4945, RSL3, BAY-299, Cotransin, MIV-6R, CP-673451, AC-4-130, LLY-507, ABPA3, TP-020, PF-4800567, Englerin A, LP99, JQEZ5, BI2536, AGI-6780, KU-60019, DS-437, BMS-265246, CMLD-2, BI-D1870, AGI-5198, WH-4-023, Cortistatin A, NI-42, BIX-01294, TX1-85-1, CFI-400945, (R)-Zinc-3573, URMC-099, XAV939, JW55, TTT20171, Imatinib, dTRIM24, MBM-55, MZP-54, TBK1 PROTAC 3i, GNE-049, WZ4002, NCT-505, SR9238, U18666A, NIK SMI1, TL13-112, GSK2982772, MD-224, LNP-023, AMG-337, MK-8033, AZD3988, RU.521, dBET6, ARS-1620, MLT-748, GDC-0834, LSN 3213128, GSK2033, PT2385, Adavosertib, VZ185, GSK2194069, MG-277, TAK-243, A-770041, GNF-5837, GSK2973980A, THAL-SNS-032, dTAG-13, GNE-781, EML631, QC6352, Capmatinib, PF-06869206, BSJ-03-123, Asciminib, SB-612111 or TH1760, TP-024.

“An approved drug” as referred herein is any chemical entity the received the U.S. Food and Drug Administration, China Food and Drug Administration, European Medicines Agency or any regulatory agency, approval for usage in human.

“A toxin” and “A cytotoxic” as referred herein is a compound with non-selective cell killing activity.

Non limiting examples of “A chemotherapeutic” include: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine or Vindesine.

“A radiolabeled probe” or “radiopharmaceuticals” include any probe or pharmaceutical, respectively which possess a radioactive isotope. Non limiting examples of radiopharmaceuticals include: 177Lu-PSMA-617 (lutetium Lu 177 vipivotide tetraxetan). 177 Lu PSMA-617 is a radiolabeled drug that target prostate-specific membrane antigen (PSMA) in prostate cancer. PSMA is a membrane bound glycoprotein which is over expressed in prostate cancer. Lutetium-177 once internalized into the cell irreversibly sequestered within the targeted tumor cell. It emits radiation over a millimeter range that is ideal for eradication of the cancer cells. The therapeutic candidate acts by binding to the PSMA expressing cancer cells and exhibit cytotoxicity. Lutetium Lu-177 dotatate or Lutetium (177Lu) oxodotreotide (Lutathera): Lutetium Lu 177 dotatate binds to somatostatin receptors with highest affinity for subtype 2 receptors (SSRT2). Upon binding to somatostatin receptor expressing cells, including malignant somatostatin receptor-positive tumors, the compound is internalized. The beta emission from Lu 177 induces cellular damage by formation of free radicals in somatostatin receptor-positive cells and in neighboring cells. Radium-223 chloride (Xofigo): The active moiety of radium Ra 223 dichloride is the alpha particle-emitting isotope radium-223, which mimics calcium and forms complexes with the bone mineral hydroxyapatite at areas of increased bone turnover, such as bone metastases. The high linear energy transfer of alpha emitters (80 keV/micrometer) leads to a high frequency of double-strand DNA breaks in adjacent cells, resulting in an anti-tumor effect on bone metastases. The alpha particle range from radium-223 dichloride is less than 100 micrometers (less than 10 cell diameters) which limits damage to the surrounding normal tissue.

In some embodiments, the compounds provided herein of formula I, IA, II, IIA or III comprise a fluorescent, a chemiluminescent or a radiolabeled probe (R₃ of formula I, IA, II, IIA or III). In other embodiments, the fluorescent probe comprises non limited examples of rhodamine, cyanine, coumarin, Nile red, Nile blue, dansyl, umberiferon, bodipy, environment sensitive fluorophore or derivative thereof. In other embodiments, the chemiluminescent probe comprises dioxetane-based compounds, 2,3-dihydrophthalazinedione such as luciferin and luminol or derivative thereof. In other embodiments the radiolabeled probe includes any ligand possessing a radioactive isotope.

In some embodiments, provided herein a compound of formula I, IA, II, IIA or III comprising a protein binding ligand (R or formula I, IA or the ibrutinib group of formula II, IIA or III), and a fluorescent, a chemiluminescent, a radiolabeled probe or a bioactive group (R₃ of formula I, IA, II, IIA or III). In another embodiment, a NH(R₂)(R₃) group and sulfur trioxide is released upon binding to the protein, while the protein binding ligand is covalently linked to the protein. In another embodiment, the covalent bond is formed via a nucleophilic moiety of the protein and the carbon directly connected to the sulfamate of the compound structures of formula I, IA, II, IIA, III. In another embodiment, the nucleophilic moiety of the protein is a thiol, an amine or a hydroxyl.

In some embodiments R₁ and R₂ of the compound of formula I, IA, II, IIA or III are each independently H, substituted or unsubstituted: linear or branched alkyl, linear or branched alkenyl, linear or branched alkynyl, aryl, alkyl aryl, cycloalkyl, heterocycloalkyl, heteroaryl.

In other embodiments, R₁ is H. In other embodiments, R₁ is substituted or unsubstituted linear alkyl. In other embodiments, R₁ is substituted or unsubstituted branched alkyl. In other embodiments, R₁ is substituted or unsubstituted linear alkenyl. In other embodiments, R₁ is substituted or unsubstituted branched alkenyl. In other embodiments, R₁ is substituted or unsubstituted linear alkynyl. In other embodiments, R₁ is substituted or unsubstituted branched alkynyl. In other embodiments, R₁ is substituted or unsubstituted aryl. In other embodiments, R₁ is substituted or unsubstituted alkyl aryl. In other embodiments, R₁ is substituted or unsubstituted cycloalkyl. In other embodiments, R₁ is substituted or unsubstituted heterocycloalkyl. In other embodiments, R₁ is substituted or unsubstituted heteroaryl.

In another embodiment, R₁ is C₁-C₆ alkyl. In another embodiment, R₁ is H or C₁-C₆ alkyl. In another embodiment, R₁ is C₁-C₃ alkyl. In another embodiment, R₁ is H or C₁-C₃ alkyl. In another embodiment, R₁ is C₁-C₆ alkyl which is unsubstituted or substituted by C₄-C₇ cycloalkyl, halo, hydroxy, alkoxy, cyano, or oxo. In another embodiment, R₁ is C₁-C₆ alkyl which is unsubstituted or substituted by C₅-C₆ cycloalkyl, halo, hydroxy, alkoxy, cyano, or oxo. In another embodiment, R₁ is unsubstituted C₅-C₆ alkyl. In another embodiment, R₁ is C₁-C₃ alkyl which is unsubstituted or substituted by C₅-C₆ cycloalkyl, halo, hydroxy, alkoxy, cyano, or oxo. In another embodiment, R₁ is C₁-C₂ alkyl which is unsubstituted or substituted by C₅-C₆ cycloalkyl, halo, hydroxy, alkoxy, cyano, or oxo. In another embodiment, R₁ is C₁-C₂ alkyl which is substituted by C₅-C₆ cycloalkyl, halo, hydroxy, alkoxy, cyano, or oxo. In another embodiment, R₁ is unsubstituted C₅-C₆ alkyl or C₁-C₂ alkyl which is substituted by C₅-C₆ cycloalkyl, halo, hydroxy, alkoxy, cyano, or oxo.

In other embodiments, R₂ is H. In other embodiments, R₂ is substituted or unsubstituted linear alkyl. In other embodiments, R₂ is substituted or unsubstituted branched alkyl. In other embodiments, R₂ is substituted or unsubstituted linear alkenyl. In other embodiments, R₂ is substituted or unsubstituted branched alkenyl. In other embodiments, R₂ is substituted or unsubstituted linear alkynyl. In other embodiments, R₂ is substituted or unsubstituted branched alkynyl. In other embodiments, R₂ is substituted or unsubstituted aryl. In other embodiments, R₂ is substituted or unsubstituted alkyl aryl. In other embodiments, R₂ is substituted or unsubstituted cycloalkyl. In other embodiments, R₂ is substituted or unsubstituted heterocycloalkyl. In other embodiments, R₂ is substituted or unsubstituted heteroaryl. In another embodiment, R₂ is C₁-C₆ alkyl. In another embodiment, R₂ is H or C₁-C₆ alkyl. In another embodiment, R₂ is C₁-C₃ alkyl. In another embodiment, R₂ is H or C₁-C₃ alkyl. In another embodiment, R₂ is C₁-C₆ alkyl which is unsubstituted or substituted by C₄-C₇ cycloalkyl, halo, hydroxy, alkoxy, cyano, or oxo. In another embodiment, R₂ is C₁-C₆ alkyl which is unsubstituted or substituted by C₅-C₆ cycloalkyl, halo, hydroxy, alkoxy, cyano, or oxo. In another embodiment, R₂ is unsubstituted C₅-C₆ alkyl. In another embodiment, R₂ is C₁-C₃ alkyl which is unsubstituted or substituted by C₅-C₆ cycloalkyl, halo, hydroxy, alkoxy, cyano, or oxo. In another embodiment, R₂ is C₁-C₂ alkyl which is unsubstituted or substituted by C₅-C₆ cycloalkyl, halo, hydroxy, alkoxy, cyano, or oxo. In another embodiment, R₂ is C₁-C₂ alkyl which is substituted by C₅-C₆ cycloalkyl, halo, hydroxy, alkoxy, cyano, or oxo. In another embodiment, R₂ is unsubstituted C₅-C₆ alkyl or C₁-C₂ alkyl which is substituted by C₅-C₆ cycloalkyl, halo, hydroxy, alkoxy, cyano, or oxo.

In some embodiments R₃ of the compound of formula I, IA, II, IIA or III is H, substituted or unsubstituted: linear or branched alkyl, linear or branched alkenyl, linear or branched alkynyl, aryl, alkyl aryl, cycloalkyl, heterocycloalkyl, heteroaryl a fluorescent probe, a chemiluminescent probe or a radiolabeled probe, a bioactive group. In other embodiments, R₃ is H. In other embodiments, R₃ is substituted or unsubstituted linear alkyl. In other embodiments, R₃ is substituted or unsubstituted branched alkyl. In other embodiments, R₃ is substituted or unsubstituted linear alkenyl. In other embodiments, R₃ is substituted or unsubstituted branched alkenyl. In other embodiments, R₃ is substituted or unsubstituted linear alkynyl. In other embodiments, R₃ is substituted or unsubstituted branched alkynyl. In other embodiments, R₃ is substituted or unsubstituted aryl. In other embodiments, R₃ is substituted or unsubstituted alkyl aryl. In other embodiments, R₃ is substituted or unsubstituted cycloalkyl. In other embodiments, R₃ is substituted or unsubstituted heterocycloalkyl. In other embodiments, R₃ is substituted or unsubstituted heteroaryl. In other embodiments, R₃ is a fluorescent probe. In other embodiments, R₃ is a chemiluminescent probe. In other embodiments, R₃ is a radiolabeled probe. In other embodiments, R₃ is a bioactive group. In another embodiment, R₃ is C₁-C₆ alkyl. In another embodiment, R₃ is H or C₁-C₆ alkyl. In another embodiment, R₃ is C₁-C₃ alkyl. In another embodiment, R₃ is H or C₁-C₃ alkyl. In another embodiment, R₃ is C₁-C₆ alkyl which is unsubstituted or substituted by C₄-C₇ cycloalkyl, halo, hydroxy, alkoxy, cyano, or oxo. In another embodiment, R₃ is C₁-C₆ alkyl which is unsubstituted or substituted by C₅-C₆ cycloalkyl, halo, hydroxy, alkoxy, cyano, or oxo. In another embodiment, R₃ is unsubstituted C₅-C₆ alkyl. In another embodiment, R₃ is C₁-C₃ alkyl which is unsubstituted or substituted by C₅-C₆ cycloalkyl, halo, hydroxy, alkoxy, cyano, or oxo. In another embodiment, R₃ is C₁-C₂ alkyl which is unsubstituted or substituted by C₅-C₆ cycloalkyl, halo, hydroxy, alkoxy, cyano, or oxo. In another embodiment, R₂ is C₁-C₂ alkyl which is substituted by C₅-C₆ cycloalkyl, halo, hydroxy, alkoxy, cyano, or oxo. In another embodiment, R₃ is unsubstituted C₅-C₆ alkyl or C₁-C₂ alkyl which is substituted by C₅-C₆ cycloalkyl, halo, hydroxy, alkoxy, cyano, or oxo. In some embodiments, R₃ is unsubstituted or substituted with halo, hydroxy, alkoxy, cyano, or oxo.

In some embodiments R₂ and R₃ of formula IA, I, IIA, II or III form together a five or six membered ring together with the nitrogen. The ring can be substituted or unsubstituted. Non limited rings include: pyridine, piperidine, pyperazine, morpholine, pyrrolidine or pyrimidine.

In some embodiments, X₁ of the compound of formula IA or IIA is a bond, N(R₁), N(R₃), O, CH₂, N(R₁)C(O), amide, ketone (C═O), ester, urea or thiourea. In other embodiments, X₁ is a bond. In other embodiments, X₁ is N(R₁). In other embodiments, X₁ is N(R₃). In other embodiments, X₁ is O. In other embodiments, X₁ is CH₂. In other embodiments, X₁ is an N(R₁)C(O). In other embodiments, X₁ is an amide (C(O)R_{y}). In other embodiments, X₁ is a ketone (C═O). In other embodiments, X₁ is an ester (C(O)OR_{y}). In other embodiments, X₁ is a urea (C(O)NRₓR_{y}). In other embodiments, X₁ is a thiourea (C(S)NRₓR_{y}). In some embodiments of the foregoing, each Rx is independently H or is selected from C₁-C₆ alkyl or C₄-C₇ cycloalkyl, each of which is unsubstituted or substituted by halo, hydroxy, alkoxy, cyano, or oxo; and each R_{y} is independently selected from C₁-C₆ alkyl or C₄-C₇ cycloalkyl, each of which is unsubstituted or substituted by halo, hydroxy, alkoxy, cyano, or oxo.

In some embodiments, X₂ of the compound of formula IA or IIA is a bond, N(R₂), N(R₃) or O. In other embodiments, X₂ is a bond. In other embodiments, X₂ is N(R₂). In other embodiments, X₂ is NR₃. In other embodiments, X₂ is O

In some embodiments, X₃ of the compound of formula IA is N(R₂)(R₃), O-alkyl, O-alkenyl, O-alkylnyl, O—CH₂CCH. In other embodiments, X₃ is N(R₂)(R₃). In other embodiments, X₃ is O-alkyl. In other embodiments, X₃ is O-alkenyl. In other embodiments, X₃ is O-alkylnyl. In other embodiments, X₃ is O—CH₂CCH.

In some embodiments, L₁, L₂, X₁ and X₂ of Formula IA are each independently a bond. In some embodiments, L₁, L₂, X₁ and X₂ of Formula IIA are each independently a bond.

In some embodiments L₁ of the compound of formula I, IA or HA is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl or an ether group. In other embodiments, L₁ is a bond. In other embodiments, L₁ is substituted or unsubstituted linear alkylene. In other embodiments, L₁ is substituted or unsubstituted branched alkylene. In other embodiments, L₁ is substituted or unsubstituted linear alkenylene. In other embodiments, L₁ is substituted or unsubstituted branched alkenylene. In other embodiments, L₁ is substituted or unsubstituted linear alkynylene. In other embodiments, L₁ is substituted or unsubstituted branched alkynylene. In other embodiments, L₁ is substituted or unsubstituted cycloalkyl. In other embodiments, L₁ is substituted or unsubstituted heterocyclic. In other embodiments, L₁ is substituted or unsubstituted aryl. In other embodiments, L₁ is substituted or unsubstituted heteroaryl. In other embodiments, L₁ is an ether group. In other embodiment, L₁ is a bond and the nitrogen is an atom within the protein binding ligand.

In some embodiments L₂ of the compound of formula I, IA, II or IIA is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl or an ether group. In other embodiments, L₂is a bond. In other embodiments, L₂ is substituted or unsubstituted linear alkylene. In other embodiments, L₂ is substituted or unsubstituted branched alkylene. In other embodiments, L₂ is substituted or unsubstituted linear alkenylene. In other embodiments, L₂ is substituted or unsubstituted branched alkenylene. In other embodiments, L₂ is substituted or unsubstituted linear alkynylene. In other embodiments, L₂ is substituted or unsubstituted branched alkynylene. In other embodiments, L₂ is substituted or unsubstituted cycloalkyl. In other embodiments, L₂ is substituted or unsubstituted heterocyclic. In other embodiments, L₂ is substituted or unsubstituted aryl. In other embodiments, L₂ is substituted or unsubstituted heteroaryl. In other embodiments, L₂ is an ether group.

In some embodiments, this invention is directed to a prodrug, wherein the prodrug comprises a Covalent Ligand Directed Releasing (CoLDR) Compound represented by the structures of Formula I, IA, II, IIA or III of this invention, wherein R is a protein binding ligand and R₃ is a drug, a targeted inhibitor, a toxin, a radiopharmaceutical or a chemotherapeutic wherein, upon interaction between a protein and the protein binding ligand, the drug or the targeted inhibitor or the toxin or the chemotherapeutic is released.

In some embodiments, provided herein a pharmaceutical composition comprising a prodrug Covalent Ligand Directed Releasing (CoLDR) Compound represented by the structures of Formula I, IA, II, IIA or III, wherein R is a protein binding ligand and R₃ is a drug, a radiopharmaceutical, a targeted inhibitor, a toxin or a chemotherapeutic and a pharmaceutical acceptable carrier.

In another embodiment, a covalent bond is formed between the protein and the protein binding ligand of the Covalent Ligand Directed Releasing (CoLDR) Compounds provided herein. In another embodiment, a covalent bond is formed via a nucleophilic moiety of the protein and carbon connect directly to the sulfamate of the CoLDR compounds provided herein. In another embodiment, the nucleophilic moiety of the protein is a thiol, an amine or a hydroxyl.

In the context of targeted covalent inhibitors, the Ibrutinib sulfamate of the structure of formula II, IIA or III and specifically compounds 3c-3e, showed similar labeling efficiency and inhibition towards BTK compared to Ibrutinib.

While BTK accommodated an electrophile switch from an acrylamide to a chloroacetamide (and sulfamate acetamide), many protein targets do not tolerate covalent binding to both types of electrophiles. Recently, many chloroacetamide inhibitors have been identified by covalent fragment screening. Chloroacetamide electrophiles may have low buffer stability and high reactivity. Hence, α-sulfamate acetamides are promising alternative for chloroacetamide inhibitors based on the fact that they maintain the same binding geometry. This will make them a useful substitution strategy in covalent medicinal chemistry.

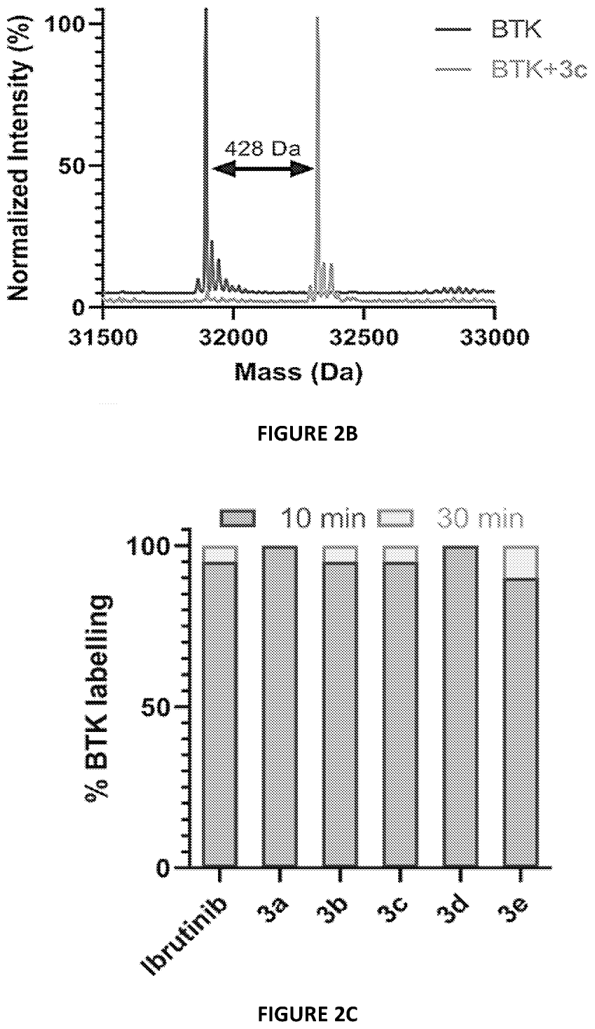

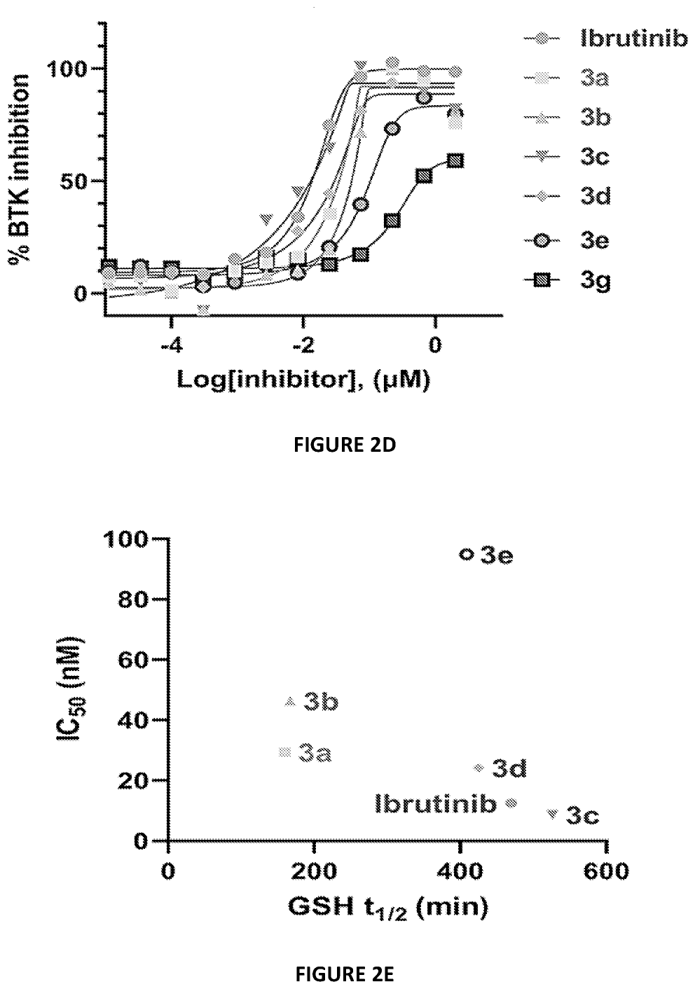

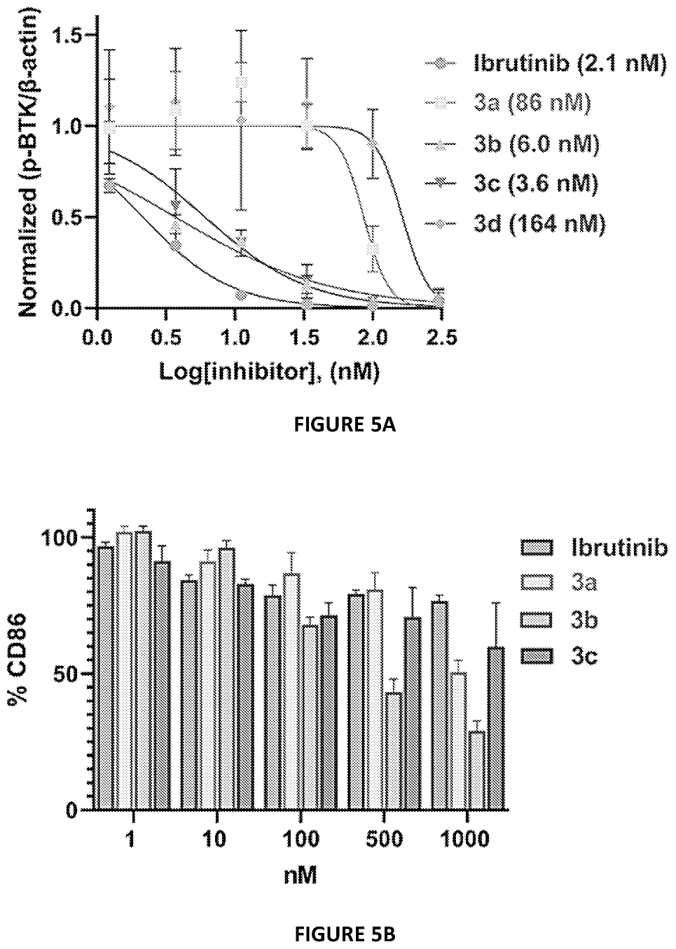

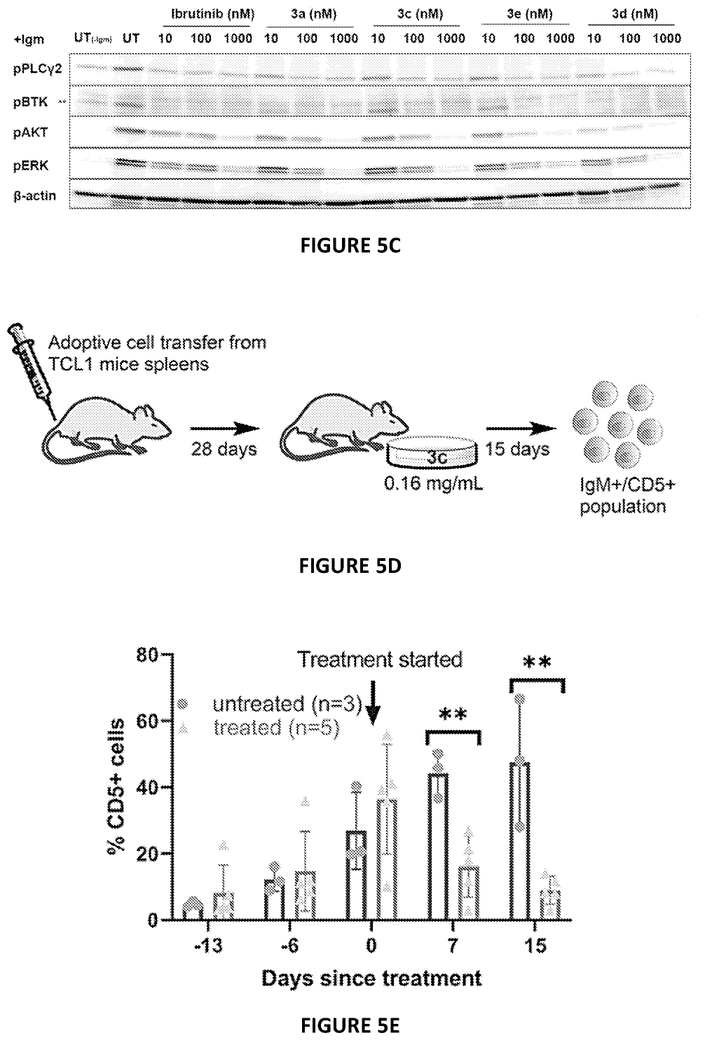

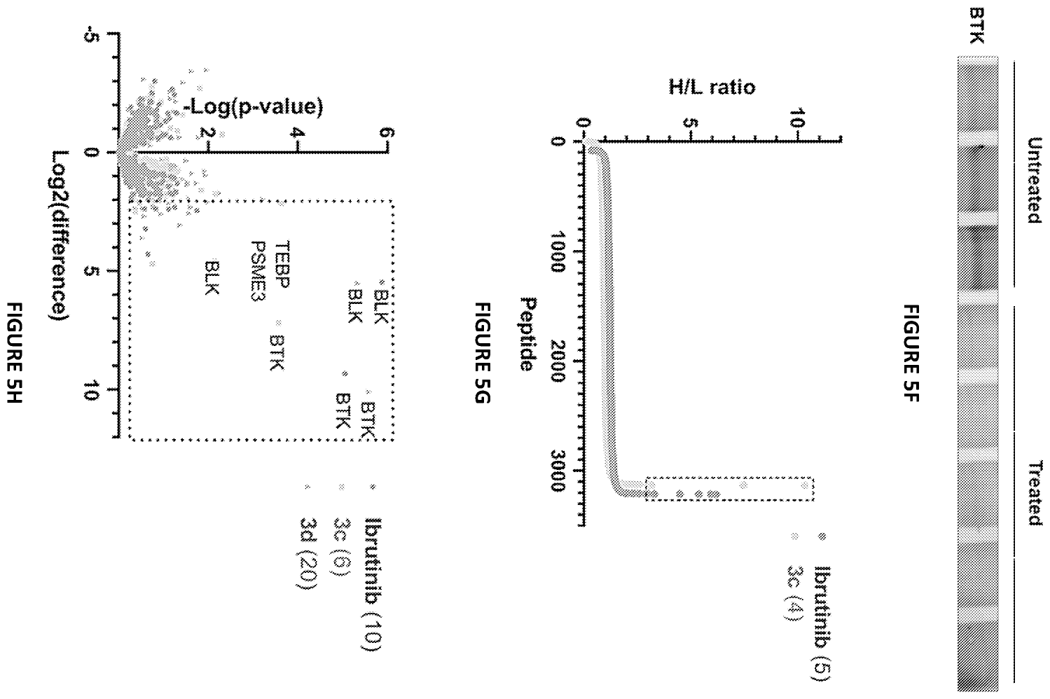

Example 2 demonstrates that the low reactivity of sulfamate acetamides (3c & 3d) showed higher kinase inhibition than chloro- and sulfonate acetamides (FIG. 2D), as well as better cellular pBTK inhibition profile than the chloroacetamide (FIG. 5A). Thus, the sulfamate group contributes to additional recognition of the protein.

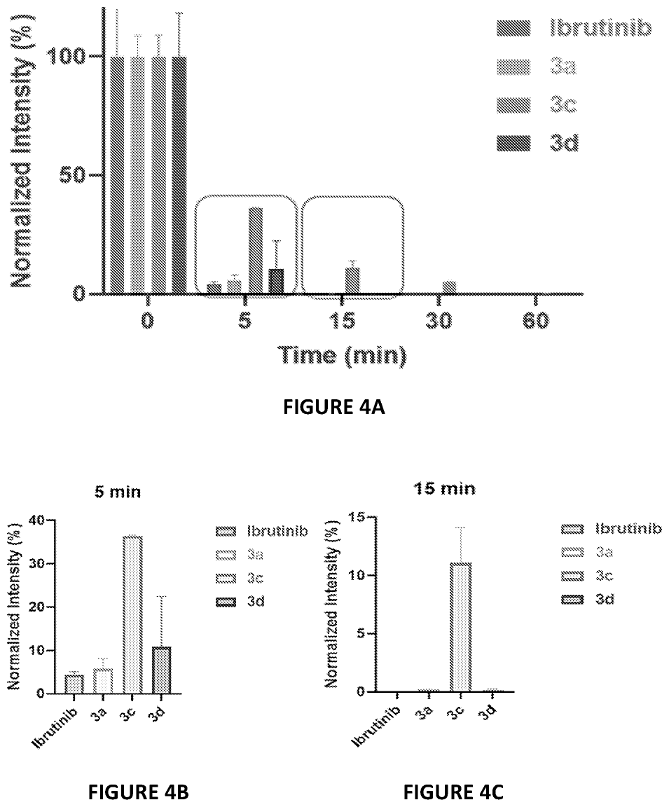

The Ibrutinib methyl sulfamate analog (3c) showed equivalent if not better performance to Ibrutinib in many settings: in vitro kinase activity assay (FIG. 2D), tissue culture, primary mouse B cells (FIG. 5A,5B) and chronic lymphocytic leukemia (CLL) patient samples (FIG. 5C), while displaying similar proteomic selectivity (FIG. 5G) and improved metabolic stability (FIGS. 4A-4C). In Example 3, when 3c was administered orally to mice with an accepted model of chronic lymphocytic leukemia (CLL), a reduction in B-cell numbers and spleen sizes was observed similar to previous reports with Ibrutinibor Acalabrutinib at the same concentration, demonstrating the oral bioavailability of this compound, as well as its suitability for in vivo administration.

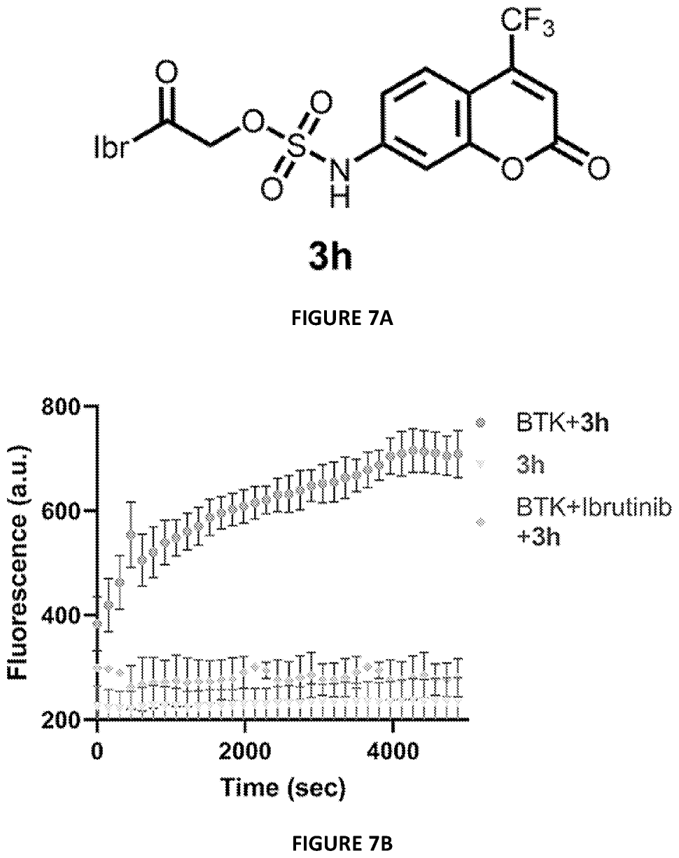

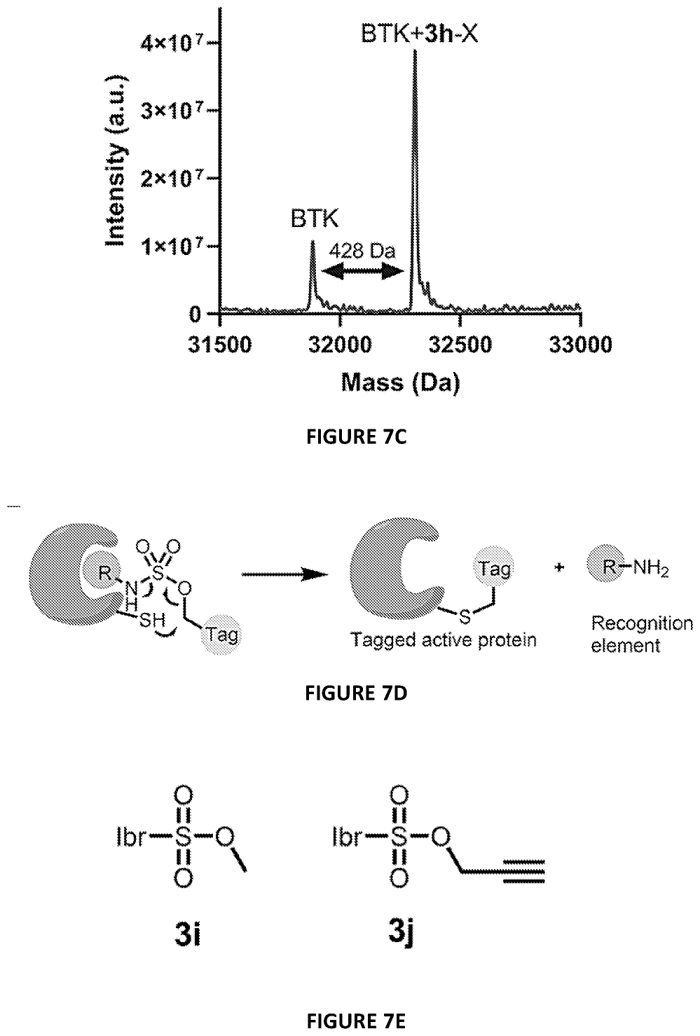

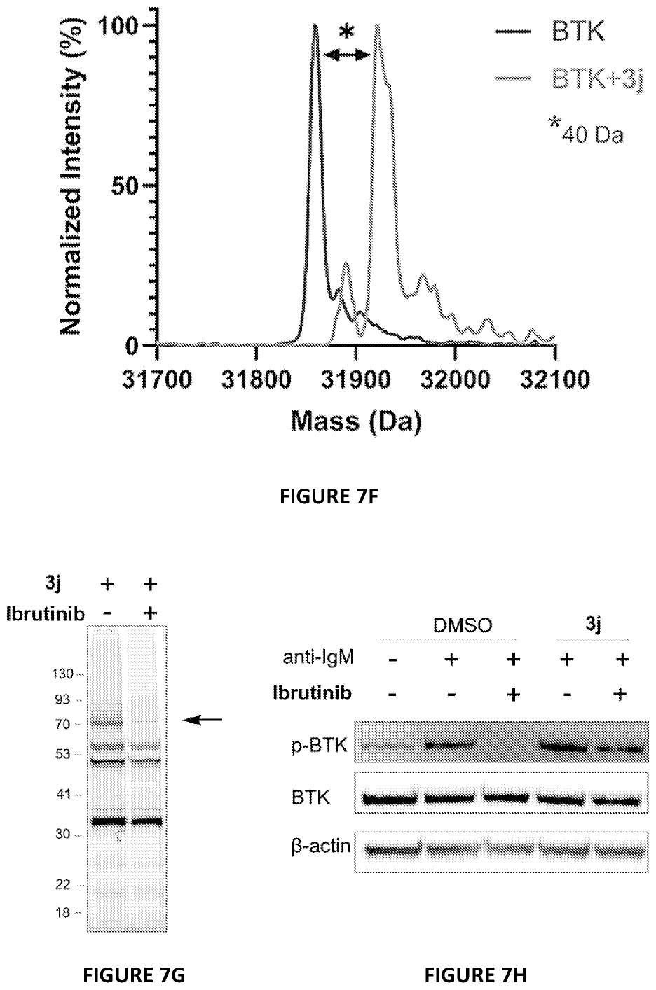

An additional advantage of the sulfamate acetamides as electrophiles provided herein is their potential for functionalization of covalent binders for various chemical biology applications. Sulfamate acetamides allow similar applications in covalent ligand directed chemistry. In the case of Ibrutinib, provided herein the release of 7-amino-4-trifluoro coumarine after reaction with BTK which resulted in enhanced fluorescence (FIG. 7B, Example 4). Since there is a wide scope of compatible leaving group functionalities, numerous potential cargoes should be available for targeted release using this strategy such as pro-drugs, and imaging agents.

Another important application of sulfamate based CoLDR chemistry is site specific labeling of proteins. The ability to functionalize an amine on a covalent inhibitor into a sulfamate allows to release said inhibitor upon covalent binding, while tagging the protein with an arbitrarily small tag.

Example 5 exemplifies site-specific labeling of proteins. Site-specific labeling of endogenous proteins concomitant with the release of a directing ligand allows the tagging of proteins in their active form. Although many ligand-directed chemistries have been reported for tagging the proteins in their apo form, they have disadvantages like targeting amino acids far from the active site, large activating groups, and less than complete control on the site of labeling. The sulfamate chemistry provided herein provides site-specific labeling of proteins where the amine group of the ligand was functionalized with a sulfo group-containing tag. When such a ligand binds to the target protein, the cysteine attacks the electrophilic carbon next to the sulfamate group and eliminates the directing ligand, leaving no, or a very minimal linker to the covalently bound tag (FIG. 7D).

Provided herein installation of a single methyl or propargyl on BTK, while preserving the protein's activity. This could be extended to installation of other functional tags such as fluorophores, bioorthogonal handles, E3 ligase recruiters, or a neo-substrate to potentially induce neo-phosphorylation by BTK.

In some embodiment, this invention provides a protein proximity inducer of a first protein and a second protein comprising a Covalent Ligand Directed Releasing (CoLDR) Compound represented by the structures of formula I, IA II, IIA or III of this invention, wherein R is a protein binding ligand for a first protein and R₃ is another protein binding ligand for a second protein, wherein, upon interaction between the second protein and the corresponding protein binding ligand, N(R₂)(R₃) is released, the second protein is then active and is labeled with R, inducing a new interaction with the first protein.

In another embodiment, a covalent bond is formed between the first protein and the corresponding protein binding ligand. In another embodiment, the covalent bond is formed via a nucleophilic moiety of the first protein and the alpha methylene between the acetamide and the sulfamate of the compounds of formula I, IA, II, IIA or III. In another embodiment, the nucleophilic moiety of the protein is a thiol, an amine or a hydroxyl.

In some embodiments, the Covalent Ligand Directed Releasing (CoLDR) Compound of this invention is used as a protein labeling to diagnose a disease or a targeted protein. The labeling of a targeted protein is done by the changes in the fluorescence or chemiluminescence or radioactivity properties upon binding of the Covalent Ligand Directed Releasing (CoLDR) Compound of this invention to the targeted protein.

In some embodiments, the Covalent Ligand Directed Releasing (CoLDR) Compound of this invention is used as a protein sensor to diagnose a disease or a targeted protein. The sensing of a targeted protein is done by the changes in the fluorescence or chemiluminescence properties or radioactivity properties if a radiolabeled probe/radiopharmaceutical is used upon binding of the Covalent Ligand Directed Releasing (CoLDR) Compound of this invention to the targeted protein.

In some embodiments, the Covalent Ligand Directed Releasing (CoLDR) Compound of this invention is used as prodrug or a drug delivery system, wherein a drug is released upon binding of the Covalent Ligand Directed Releasing (CoLDR) Compound of this invention to a targeted protein.

In some embodiments, the Covalent Ligand Directed Releasing (CoLDR) Compound of this invention is used for protein proximity inducer wherein R of formula I is a protein binding ligand for the first protein and N(R₂)(R₃) constitute or are a part of another protein binding ligand another protein binding ligand for the second protein, wherein, upon interaction between the second protein and the its protein binding ligand, SO₃N(R₂)(R₃) is released, and the second protein is then active and is labeled with R, inducing a new interaction with the first protein.

The prodrugs, drug delivery system, protein sensor, protein proximity inducer or protein labeling of this invention offer several advantages for drug discovery and chemical biology including, predictable attenuation of reactivity, late-stage installation with no additional modifications to the core scaffold, and importantly the ability to functionalize compounds as turn-on probes.

Using the covalent ligand directed release (CoLDR) chemistry provided herein, various potential drug targets like BTK, KRAS, SARS-Cov-2-PLpro were modified with different probes. For BTK selective labelling in cells were shown of both alkyne and fluorophores tags. Protein labelling by traditional affinity methods often inhibits protein activity since the directing ligand permanently occupies the target binding pocket. Using CoLDR chemistry, modification of BTK by the probes provided herein in cells preserves its activity. Further, the half-life of drug targets (such as BTK) in its native environment with minimal perturbation is being determined using the Covalent Ligand Directed Releasing (CoLDR) Compound structures of this invention. Using an environment-sensitive ‘turn-on’ fluorescent probe, the ligand binding to the active site of drug targets (such as BTK) is monitored. In another embodiment the efficient degradation of BTK by CoLDR-based BTK PROTACs (DC₅₀<100 nM), which installed a E3 ligase binder target (e.g. CRBN binder) onto BTK is provided. In another embodiment provided herein an efficient degradation of a protein target by CoLDR-based PROTACs are provided by installing an E3 ligase binder covalently on the target. This type of Proteolysis targeting chimeras (PROTACs) may enable the tuning of degradation kinetics of the target protein while keeping the protein in its active form. This approach joins very few available labeling strategies that maintain the target protein activity and thus makes an important addition to the toolbox of chemical biology.

In some embodiments, the compounds or probes disclosed herein are used to label proteins (non-limiting examples include: BTK, KRAS, and SARS-COV-2-PLpro) to their active site (having hydroxyl, thiol or amine groups). This site-selective labeling comes with many advantages like the development of “turn on” fluorescent probes, half-life identification in the native cellular environment, and PROTACs (Proteolysis targeting chimeras) for degradation.

In some embodiments, the compounds/probes disclosed herein are used for ligand-directed chemistry—for the identification of off-targets of potential covalent inhibitors or for imaging experiments. As these compounds are derived from their corresponding covalent inhibitors, no optimization of linker length is required to label the same functional group (i.e thiol of the cysteine). The importance of these probes is that they don't inhibit the activity of the native protein and their downstream signals after labeling with activity probes. This allows to study the properties of the protein in the native cellular environment.

In some embodiments, the compounds/probes disclosed herein are used for labeling an environmentally sensitive dye (i.e. Nile red) to a protein (i.e. BTK) as a turn-on fluorescent probe, which shows an improvement in the fluorescent intensity. Since environmental sensitive probes give information of the protein structure, and the presence of ligands could change its structure, this method helps to find the structure of the protein in the absence of the ligand. Further, the lack of ligand in the active site keeps the protein active with turn-on fluorescence.

In some embodiments, the compounds/probes disclosed herein are used to find the half-life of a protein in its native cellular environment without interfering with the other biological processes. Several methods like pulse-chase radiolabeling assay and cycloheximide (CHX) assay for the identification of half-life of the protein have been reported. The main disadvantage of the pulse-chase assay is that it includes many steps that can be time-consuming and requires radiolabeling. Furthermore, cycloheximide changes the cellular process by stopping the synthesis of all the proteins. The compounds/probes disclosed herein do not change half-life in cycloheximide assay whereas Ibrutinib reduces its half-life by two hours. The modification of protein half life without affecting its activity may be possible with different functional moieties like PEG linkers, or hydrophobic degraders.

In some embodiments, the compounds/probes disclosed herein are used for the degradation of a protein (i.e BTK) using PROTACs, wherein the covalently attached E3 ligase binder (i.e. CRBN binder) to the protein without the ligand degrades it efficiently. This method could help to tune the protein degradation kinetics without affecting its activity.

In some embodiments, the compounds/probes disclosed herein are used for labeling proteins in native cellular environment which upon labeling releases the ligand thereby stays active. This method enables various applications like half-life identification and targeted degradation of proteins.

In some embodiments, the compounds/probes disclosed herein allow the site-specific cellular labeling of a native protein of interest while sparing its enzymatic activity.

The advantage of the compounds/probes disclosed herein is that there is no need to change the position of the electrophilic carbon, minimizing the risk of interfering with covalent bond formation to the target. It also means that it is known apriori which residue will be labeled with the newly installed tag.

In some embodiments, the use of the compounds/probes disclosed herein for labeling platform provides an environment-sensitive ‘turn-on’ fluorescent probe. In addition to the generation of fluorescence upon binding, the active protein is labeled, and the dye can serve as a reporter for binding events in the protein and perhaps for its conformation. The fact that probes provided herein do not hinder binding to the active site, can facilitate investigation of alternative ligands binding events.

Provided herein, a new platform for site-specific labeling of proteins, that is compatible with cellular conditions and spares the labeled protein's activity. This approach joins very few such available strategies and thus makes an important addition to the toolbox of chemical biology.

As used herein, the term alkyl, used alone or as part of another group, refers, in one embodiment, to a “C1 to C18 alkyl” and denotes linear and branched, saturated or unsaturated (e.g., alkenyl, alkynyl) groups, the latter only when the number of carbon atoms in the alkyl chain is greater than or equal to two, and can contain mixed structures. Non-limiting examples are alkyl groups having from 1 to 6 carbon atoms (C1 to C6 alkyls), or alkyl groups having from 1 to 4 carbon atoms (C1 to C4 alkyls). Examples of saturated alkyl groups include, but are not limited to, methyl, ethyl, n-propyl, isopropyl, n-butyl, iso-butyl, sec-butyl, tert-butyl, amyl, tert-amyl and hexyl. Examples of alkenyl groups include, but are not limited to, vinyl, allyl, butenyl and the like. Examples of alkynyl groups include, but are not limited to, ethynyl, propynyl and the like. Similarly, the term “C1 to C18 alkylene” denotes a bivalent radical of 1 to 18 carbons.

When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, amino halogen, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio or alkylsulfonyl groups. Any substituents can be unsubstituted or further substituted with any one of these aforementioned substituents.

Herein, the term “alkenyl” describes an unsaturated aliphatic hydrocarbon comprise at least one carbon-carbon double bond, including straight chain and branched chain groups. Optionally, the alkenyl group has 2 to 20 carbon atoms, or, the alkenyl is a medium size alkenyl having 2 to 10 carbon atoms, or the alkenyl is a lower alkenyl having 2 to 4 carbon atoms. The alkenyl group may be substituted or non-substituted.

Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Optionally, the alkynyl group has 2 to 20 carbon atoms, or, the alkynyl is a medium size alkynyl having 2 to 10 carbon atoms, or the alkynyl is a lower alkynyl having 2 to 4 carbon atoms. The alkynyl group may be substituted or non-substituted.

Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

As used herein, “alkylene” refers to a linear, branched or cyclic, in certain embodiments linear or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)2 groups, or substituted or unsubstituted nitrogen atoms including —NR— and —N+RR-groups, where the nitrogen substituent(s) is(are) alkyl, aryl, aralkyl, heteroaryl, heteroaralkyl or COR, where R is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH2), ethylene (—CH2CH2-), propylene (—(CH2)3-), methylenedioxy (—O—CH2-O—) and ethylenedioxy (—O—(CH2)2-O—). The term “lower alkylene” refers to alkylene groups having 1 to 6 carbons. In certain embodiments, alkylene groups are lower alkylene, including alkylene of 1 to 3 carbon atoms.

As used herein, “alkenylene” refers to a linear, branched or cyclic, in one embodiment straight or branched, divalent aliphatic hydrocarbon group, in certain embodiments having from 2 to about 20 carbon atoms and at least one double bond, in other embodiments I to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —H═CH—CH2. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, a divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH2-. The term “lower alkynylene” refers to alkynylene groups having 2 to 6 carbons. In certain embodiments, alkynylene groups are lower alkynylene, including alkynylene of 3 to 4 carbon atoms.

The term “aryl” used herein alone or as part of another group denotes an aromatic ring system having from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups such as halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl —OCN, —SCN, —N═C═O, —NCS, —NO, —N3, —OP(═O)(OR*)2, —P(═O)(OR*)2, —P(═O)(O—)2, —P(═O)(OH)2, —P(O)(OR*)(O—), —C(═O)R*, —C(═O)X, —C(S)R*, —C(S)OR*, —C(O)SR*, C(S)SR*, —C(S)NR*2 or —C(═NR*)NR*2 groups, where each R* is independently H, alkyl, aryl, arylalkyl, a heterocycle, or a protecting group or prodrug moiety groups. Any substituents can be unsubstituted or further substituted with any one of these aforementioned substituents.

The term “heteroaryl” refers to an aromatic ring system containing from 5-14 member ring having at least one heteroatom in the ring. Non-limiting examples of suitable heteroatoms which can be included in the aromatic ring include oxygen, sulfur, phospates and nitrogen. Non-limiting examples of heteroaryl rings include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc. The heteroaryl group can be unsubstituted or substituted through available carbon atoms with one or more groups such as. halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, amido, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl, —OCN, —SCN, —N═C═O, —NCS, —NO, —N3, —OP(═O)(OR*)2, —P(═O)(OR*)2, —P(═O)(O-)2, —P(═O)(OH)2, —P(O)(OR*)(O—), —C(═O)R*, —C(═O)X, —C(S)R*, —C(S)OR*, —C(O)SR*, C(S)SR*, —C(S)NR*2 or —C(═NR*)NR*2 groups, where each R* is independently H, alkyl, aryl, arylalkyl, a heterocycle, or a protecting group or prodrug moiety. Any substituents can be unsubstituted or further substituted with any one of these aforementioned substituents.

The invention includes “pharmaceutically acceptable salts” of the compounds of this invention, which may be produced, by reaction of a compound of this invention with an acid or base. Certain compounds, particularly those possessing acid or basic groups, can also be in the form of a salt, optionally a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

The terms “compound” and “probe” are used herein interchangeably.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Various embodiments and aspects of the present invention as delineated hereinabove and as claimed in the claims section below find experimental support in the following examples.

### EXAMPLES

### Methods for Experiments Described in Examples 1-7.

### LC/MS Measurements

LC/MS runs were performed on a Waters ACQUITY UPLC class H instrument, in positive ion mode using electrospray ionization. UPLC separation for small molecules used C18-CSH column of (1.7 μm, 2.1 mm×100 mm) for all the LC/MS based assays. The column was held at 40° C. and the autosampler at 10° C. Mobile phase A was 0.1% formic acid in the water, and mobile phase B was 0.1% formic acid in acetonitrile. The run flow was 0.3 mL/min. The gradient used was 100% A for 2 min, increasing linearly to 90% B for 5 min, holding at 90% B for 1 min, changing to 0% B in 0.1 min, and holding at 0% for 1.9 min. UPLC separation for proteins used a C4 column (300 Å, 1.7 μm, 2.1 mm×100 mm). The column was held at 40° C. and the autosampler at 10° C. Mobile solution A was 0.1% formic acid in the water, and mobile phase B was 0.1% formic acid in acetonitrile. The run flow was 0.4 mL/min with gradient 20% B for 2 min, increasing linearly to 60% B for 3 min, holding at 60% B for 1.5 min, changing to 0% B in 0.1 min, and holding at 0% for 1.4 min. The mass data were collected on a Waters SQD2 detector with an m/z range of 2-3071.98 at a range of m/z of 800-1500 Da for BTK.

### GSH Reactivity Assay for Model Compounds and Ibrutinib Sulfamates

A 100 μM (for 3a-3e) 200 μM (for 1a-1 h) (0.5 μL of 20 mM stock) sample of the electrophile was incubated with 5 mM GSH (5 μL of 100 mM stock, freshly dissolved), 5 mM NaOH (to counter the acidity imparted by GSH) and 100 μM 4-nitrocyano benzene (0.5 μl of 20 mM stock solution) as an internal standard in 100 mM potassium phosphate buffer pH 8.0 and DMF at a ratio of 9:1, respectively. All solvents were bubbled with argon. Reaction mixtures were kept at 10° C. Every 1 h, 5 μL from the reaction mixture were injected into the LC/MS. The reaction was followed by the peak area of the electrophile normalized by the area of the 4-nitrocyano benzene (i.e. by the disappearance of the starting material). The natural logarithm of the results was fitted to linear regression, and t_{1/2} was calculated as t_{1/2}=ln 2/−slope.

### DTNB Thiol Reactivity Assay

The compound 5,5′-dithio-bis(2-nitrobenzoic acid) (DTNB; 50 μM) was incubated with 200 μM tris(2-carboxyethyl)phosphine (TCEP) in 20 mM sodium phosphate buffer pH 7.4 and 150 mM NaCl for 5 min at room temperature, to obtain TNB⁻². Next, 20 μM compounds were subsequently added to TNB⁻² followed by immediate ultraviolet (UV) absorbance measurement at 412 nm and 37° C. UV absorbance was acquired every 15 min for 7 h. The assay was performed in a 384-well plate using a Tecan Spark 10M plate reader. Background absorbance of compounds was subtracted by measuring the absorbance at 412 nm of each compound under the same conditions without DTNB. Compounds were measured in triplicate. The data were fitted to a second-order reaction equation such that the rate constant (K) is the slope of ln([A][B0]/[B][A0]), where [A0] and [B0] are the initial concentrations of the compound (200 μM) and TNB⁻² (100 μM), respectively, and [A] and [B] are the remaining concentrations as a function of time as deduced from spectrometric measurements. Linear regression using Prism was performed to fit the rate against the first 7 h of measurements.

### Buffer Stability Assay for Model Compound, Ibruinib Sulfamates

A sample of the electrophile (200 μM for 1a-1i and 100 μM for 3a-3g) was incubated with 100 μM of 4-nitrocyano benzene as an internal standard in a 100 mM potassium phosphate buffer of pH 8.0. Reaction mixtures were kept at 37° C. with shaking. After 4 days (unless otherwise mentioned), 5 μL from the reaction mixture were injected into the LC/MS to check the stability of the compounds.

### In-Gel Fluorescence Labeling Profile

Mino cells were cultured in RPMI-medium supplemented with 15% FBS and 1% p/s, at 37° C. and 5% CO₂. The cells were treated for 2 h with either 0.1% DMSO or the indicated concentrations of 2a-2c or 3j. For the competition experiment, the cells were pre-incubated for 30 min with 1 μM Ibrutinib followed by 2 h incubation with 100 nM 3j. The cells were lysed with RIPA buffer (Sigma, R0278) and protein concentration was determined using BCA protein assay (Thermo Fisher Scientific, 23225). Lysates were then diluted to 2 mg/mL in PBS. Lysates were clicked to TAMRA-azide (Lumiprobe). “Click” reaction was performed using a final concentration of 4 μM TAMRA-azide, 3 mM CuSO₄, 3 mM Tris(3-hydroxypropyltriazolylmethyl)amine (THPTA, Sigma), and 3.7 mM Sodium L-ascorbate (Sigma) in a final volume of 60 μL. The samples were subjected to precipitation.

Precipitation: lx chloroform, 4× methanol, and 3× water were added to the samples and vortexed thoroughly. The samples were spun down for 10 minutes at 4° C. The top layer was aspirated and the pellet was resuspended in 4× methanol. The sample was vortexed and spun down again for 10 min at 4° C. and removed the solution and dried the pellet for 2 minutes. The pellet was dissolved in 42 μL PBS followed by a 14 μL of 4× sample buffer. The samples were then loaded on a 4-20% Bis-Tris gel (SurePAGE, GeneScript) and imaged at 532 nm using Typhoon FLA 9500 scanner.

### In-Vitro Labeling Experiments

BTK kinase domain was expressed and purified as previously reported [Gabizon, R. et al. Efficient Targeted Degradation via Reversible and Irreversible Covalent PROTACs. J. Am. Chem. Soc. (2020) doi:10.1021/jacs.9b13907 which is incorporated herein by reference]. Binding experiments were performed in Tris 20 mM pH 8.0, and 50 mM NaCl at room temperature. The BTK kinase domain was diluted to 2 μM in the buffer, and 2 μM Ibrutinib derivatives (3a-3g) were added by adding 1/100th volume from a 200 μM solution.

The reaction mixtures, at room temperature for various times, were injected into the LC/MS. For data analysis, the raw spectra were deconvoluted using a 20000:40000 Da and 1 Da resolution. The labeling percentage for a compound was determined as the labeling of a specific compound (alone or together with other compounds) divided by the overall detected protein species.

### In-Vitro Kinase Activity

Kinase reactions are assembled in 384-well plates (Greiner) in a total volume of 20 μL. Test compounds (3a-3g) were diluted in DMSO to a final concentration, while the final concentration of DMSO in all assays was kept at 1%. The compounds were incubated with the kinases for 30 min. A 0.5 nM concentration of BTK in 100 mM HEPES, pH 7.5; 0.1% BSA, 0.01% Triton X-100, 1 mM DTT, 5 mM MgCl₂, were used. The reaction was initiated by 2-fold dilution into a solution containing 5 μM ATP and 1 μM substrate in the kinase buffer.

### BTK Activity in Mino Cells

Mino cells were treated with indicated concentration of the compounds (3a-g, 3j). The cells were then incubated with 10 μg/mL anti-human IgM (Jackson ImmunoResearch, 109-006-129) for 10 min at 37° C. and harvested. The cell pellets were subjected to immunoblotting and performed Western blots for p-BTK, BTK and β-actin.

### Immunoblotting

Cell pellets were washed with ice-cold PBS and lysed using RIPA-buffer (Sigma, R0278). Lysates were clarified at 21,000 g for 15 min at 4° C. and protein concentration was determined using BCA protein assay (Thermo Fisher Scientific, 23225). Samples containing 50 g total protein were prepared with 4×LDS sample buffer (NuPAGE, Thermo Scientific, NP0007) and 20 mM DTT which were then resolved on a 4-20% bis-tris gel (GeneScript SurePAGE, M00657). Proteins were separated by electrophoresis and were then transferred to a nitrocellulose membrane (Bio-Rad, 1704158) using the Trans-Blot Turbo system (Bio-Rad). The membrane was blocked with 5% BSA in TBS-T (w/v) for 1 h at room temperature, washed ×3 times for 5 min with TBS-T and incubated with the following primary antibodies: rabbit anti phospho-BTK (#87141s, cell-signaling, 1:1000, over-night at 4° C.), mouse anti BTK (#56044s, cell-signaling, 1:1000, 1 h at room-temperature), mouse anti β-actin (#3700, cell-signaling, 1:1000, 1 h at room-temperature). Membrane was washed ×3 times for 5 min with TBS-T and incubated with the corresponding HRP-linked secondary antibody (Mouse #7076/Rabbit #7074, cell-signaling) for 1 h at room-temperature. EZ-ECL Kit (Biological Industries, 20-500-1000) was used to detect HRP-activity. The membrane was stripped using Restore stripping buffer (Thermo Fisher Scientific, 21059) after each secondary antibody before blotting with the next one.

### B-Cell Response Experiment

Splenic cells from C57BL/6 mice were isolated by forcing spleen tissue through the mesh into PBS containing 2% fetal calf serum and 1 mM EDTA and red blood cells were depleted by lysis buffer. Cells were cultured in 96-well U-bottom dishes (1×10⁶ cells/mL in RPMI 10% FCS) and incubated with Ibrutinib, IbrCl-1342 in different concentrations (1 nM, 10 nM, 100 nM, 1000 nM) for 24 h at 37° C. in 5% humidified CO₂. Following a 24 h incubation, cells were stimulated with anti-IgM overnight (5 g/mL, Sigma-Aldrich). Subsequently, cells were stained with anti-B220 (clone RA3-6B2, Biolegend) and anti-CD86 (clone GL-1, Biolegend) antibodies (anti-mouse CD86 biolegend 105008 1:400, anti-mouse/human CD45R/B220 biolegend 103212 1:400) for 30 min at 4° C. Single-cell suspensions were analyzed by a flow cytometer (CytoFlex, Beckman Coulter).

### BTK Activity in CLL Patient Samples

CLL cells (20×10⁶/mL) were incubated with Ibrutinib or Ibrutinib-based compounds (3a, 3c-3e), at the indicated doses at 37° C. DMSO treated cells served as controls. After 2 hours of incubation, the cells were stimulated with goat F(ab′)2 anti-human IgM (10 μg/mL) for 15 minutes or left untreated. CLL cells were lysed in RIPA lysis buffer (Cell Signaling Technology, Beverly, MA) containing phosphatase inhibitor cocktail 2 and protease inhibitor cocktail (Sigma-Aldrich, MO, USA). Extract from cell lysates were separated on 4-15% Criterion™ TGX™ Precast Midi Protein Gel (Bio-Rad Laboratories) and transferred electrophoretically to nitrocellulose membrane (Bio-Rad Laboratories). The membranes were incubated with the designated antibodies and HRP conjugated secondary antibodies according to the manufacturer's instructions. Bands were detected using MyECL Imager (Thermo Scientific, Rockford, TL). A Western blot analysis showed PLCγ2, BTK, Akt, and ERK phosphorylation as well as the total amount of these proteins. Actin was used to verify equal loading. More details available in Supplementary information.

### IsoDTB ABPP Sample Preparation

The probe peptide was synthesized using standard solid phase synthesis on rink amide resin. The resin was swelled in dichloromethane for 30 minutes, washed with DMF, and deprotected using 20% piperidine/DMF (3×5 minutes). 2 equivalents Fmoc-(azidolysine)-OH were coupled in DMF using 2 equivalents of HATU and 4 equivalents of diisopropylethylamine for 2 hours with tumbling, followed by 3 washes with DMF and fmoc deprotection using the same method used above. At this step, 2 equivalents of fmoc-Val-OH (for the light probe) or fmoc-Val-OH (13C5, 99%, 15N, 99%; Cambridge isotope laboratories) were coupled using the same method as before, followed by fmoc deprotection. This was followed by coupling to 2 equivalents of desthiobiotin (using the same method), followed by 3 washes with DMF, 3 washes with dichloromethane, and drying in a vacuum desiccator.

The peptides were cleaved from the resin using 95% TFA, 2.5% TIPS and 2.5% water for 3 hours, followed by thorough evaporation of the cleavage mixture using nitrogen bubbling and purification by reverse phase HPLC. The purified peptides were dissolved in DMSO to a concentration of 5 mM and used directly.

The preparation of IsoDTB-ABPP samples was performed essentially as described in Zanon et al. [Zanon, P. R. A., et al, Isotopically Labeled Desthiobiotin Azide (isoDTB) Tags Enable Global Profiling of the Bacterial Cysteinome. Angew. Chem. Int. Ed Engl. 2020, 59 (7) 2829-2836 which is incorporated herein by reference] which is incorporated by reference. Experiments were conducted in quadruplicates. PATU cells were incubated for 2 h with 5 μM compounds 3g (or with DMSO), and collected by centrifuge at 300 g for 5 min followed by ice cold PBS wash. For lysis, samples containing 10 million cells were dispersed in 0.5 mL of RIPA buffer (Sigma, R0278), incubated with occasional vortexing for 30 min on ice, followed by centrifugation at 21,000 g for 15 min. The protein concentration in the samples was determined using BCA assay (Pierce 23227), and each sample was diluted to 1.7 mg/mL using PBS. To each sample, 5 μL of 10 mM IA-alkyne was added, followed by 1 h incubation at room temperature in the dark. 10 μL of 5 mM DesThioTag was added (Light for the compound treated samples, heavy for the DMSO-treated samples), followed by 18 μL of CuSO4:TTHPTA (100 mM), and the click reaction was initiated by addition of 15 μL of 150 mM sodium ascorbate (freshly dissolved in water). The samples were incubated on a rotary shaker for 1 h at room temperature. The compound-treated and DMSO-treated samples were mixed with 4 mL methanol, 1 mL chloroform and 2 mL water on ice, vortexed and centrifuged at 3200 g for 10 min at 4° C. The top layer was aspirated, and 3 mL methanol was added, followed by centrifugation and aspiration of the supernatant. The pellets were air dried and stored at −80° C. until the following treatment.

### Pull-Down Proteomics Experiments

Mino cells were incubated for 1 h with DMSO, Ibrutinib, 3c and 3d followed by the incubation with 10 μM “probe 4” for another hour. The cells were lysed and “clicked” with biotin-azide (Click Chemistry Tools, CAT 1265) and the samples were incubated at room temperature for 1 hour. The samples were then precipitated with methanol: chloroform (1 mL methanol, 250 μL chloroform, 750 μL water), washed with 1 mL of methanol and air-dried. The samples were solubilized and bound to streptavidin agarose beads in PBS for 3 h at 25° C. The beads were washed, centrifuged, and resuspended in Tris 50 mM pH 8 and transferred to a clean Eppendorf tube. After this, the bound proteins were eluted by boiling with 5% SDS then reduced with DTT, alkylated with iodoacetamide, and digested with trypsin. The samples were run on LC/MS/MS. The detailed procedure is available in the supplementary methods section.

### μTCL1 Adoptive Transfer Model

TCL1 mice for this model were generated as previously described. [Hofbauer, J. P. et al. Development of CLL in the TCL1 transgenic mouse model is associated with severe skewing of the T-cell compartment homologous to human CLL. Leukemia 2011, 25 (9) 1452-1458 which is incorporated herein by reference]. For this experiment, TCL1 mice approximately 12 months of age, with a malignant cell population higher than 60% in the PB were sacrificed. Their spleens were excised, and 4×10⁷ cells resuspended in PBS^{-/-} were injected into the tail vein of 6-weeks-old recipient mice. Progression of the disease was followed in the PB by using flow cytometry for the +IgM/+CD5 population. Mice with >30% IgM+/CD5+ cells were considered to be diseased and were used for further analysis.

### Staining for Flow Cytometry.

Isolated cells were stained using specific antibodies (IgM-PE, CD5-APC, BioLegend®) in staining buffer (0.5% bovine serum albumin in phosphate-buffered saline) for 30 min in 4° C. in dark then washed twice. Flow cytometry (FACS) analysis was performed using FACS Canto (BD Biosciences) and data were collected using FACSDIva8 (BD Biosciences). FACS data analysis was done using Flowjo v10.

### BTK Engagement in Treated Mice Spleens

Pellets of harvested spleens were lysed using RIPA buffer (Sigma, R0278), clarified at 21,000 g for 15 minutes at 4° C. and protein concentration was determined using BCA protein assay (Thermo Scientific, 23225). Lysates were diluted to 2 mg/ml, 50 ml per sample, and incubated for 1 h in room temperature with 1 mM probe 4 to label BTK. Lysates were then clicked to TAMRA-azide and imaged using ChemiDoc MP (546 nm) as described in the In-gel fluorescence protocol.

### Example 1

Synthesis of Compounds of this Invention.

Materials and methods: All reagents and solvents were obtained from commercial suppliers unless otherwise mentioned. Ibr-H (CAS 1022150-12-4) was purchased from BLD pharmatech. Deuterated solvents were purchased from Cambridge isotope laboratories and all other reagents are purchased from Sigma Aldrich and used as such without further purification.

Aluminum-backed silica plates (Merck silica gel 60 F254) were used for thin layer chromatography (TLC) to monitor solution phase reactions. The purification of compounds was carried out on a combi flash chromatography and waters RP-HPLC with Prep C18 column. All the compounds used in the reactivity assays/cellular assays were waters RP-HPLC with Prep C18 column. The ¹H-NMR and ¹³CNMR spectra were recorded using a 400 MHz and 500 MHz Bruker advance spectrometers and were calibrated using residual undeuterated solvent as the internal references (CDCl₃: 7.26:; DMSO-d6: 2.50:; D₂O: 4.79; and CD₃OD=3.31:). Chemical shifts are reported in: on a δ scale. The following abbreviations were used to explain NMR peak multiplicities: s=singlet, d=doublet, t=triplet, q=quartet, p=pentet, m=multiplet, br=broad. Most of the Ibrutinib derivatives appeared as a mixture of rotamers. The high-resolution mass spectra were recorded on Waters Xevo G2-XS QTof mass spectrometer using electrospray ionization time-of-flight (ESI-TOF) reflectron experiments.

### Synthesis of (R)-3-(4-phenoxyphenyl)-1-(piperidin-3-yl)-1H-pyrazolo[3,4-d]pyrimidin-4-amine (Ibrutinib)

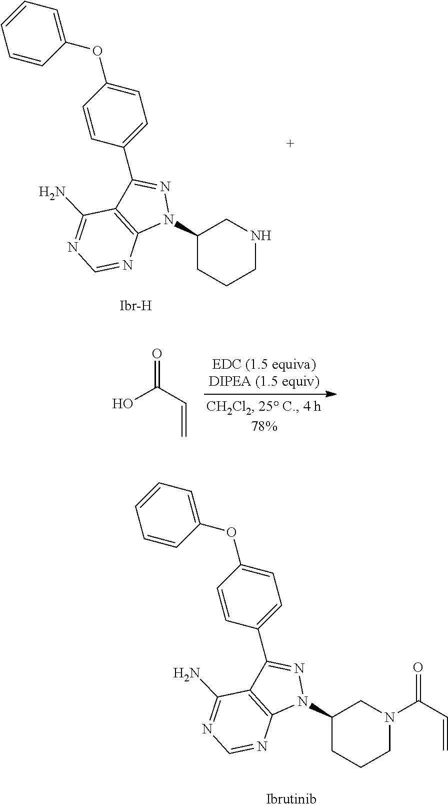

To a stirred solution of acrylic acid (1.02 mL, 15 mmol) in anhydrous CH₂Cl₂ (50 mL), N-(3-dimethylaminopropyl)-N′-ethylcarbodiimide hydrochloride (EDC·HCl) (2.88 g, 15 mmol), N,N-Diisopropylethylamine (2.60 mL, 15 mmol) and Ibr-H (3.87 g, 10 mmol) were added at 0° C. under N₂ atmosphere. The reaction mixture was stirred at room temperature for 4 h. After completion (as monitored by LC-MS), of the reaction, H₂O (30 mL) was added. The organic layer was extracted with CH₂Cl₂ (3×50 mL) and evaporated under vacuo. The crude product was purified by column chromatography over silica gel using EtOAc:MeOH (9:1)/Pet. ether as eluent to give pure Ibrutinib as colorless solid 3.47 g (yield=78%). This compound is reported in the literature.³

¹H NMR (500 MHz, CD₃OD) (as a mixture of rotamers) δ 1.67-1.78 (m, 1H), 2.04-2.15 (m, 1H), 2.26 (dd, J=12.7, 3.6 Hz, 1H), 2.33-2.44 (m, 1H), 3.26 (t, J=10.4, 0.4H) (t, J=11.3 Hz, 0.6H), 3.58 (dd, J=12.2, 10.2 Hz, 0.6H), 3.88 (m, 0.4H), 4.05 (d, J=13.6 Hz, 0.6H), 4.23 (m, 0.8H), 4.56 (d, J=12.0 Hz, 0.6H), 4.88 (m, 1H), 5.76 (d, J=10.7 Hz, 1H), 6.11-6.23 (m, 1H), 6.81 (dd, J=16.7, 10.7 Hz, 1H), 7.10 (d, J=7.7 Hz, 2H), 7.15 (d, J=8.7 Hz, 2H), 7.18 (t, J=7.4 Hz, 1H), 7.41 (t, J=8.0 Hz, 2H), 7.68 (d, J=8.7 Hz, 2H), 8.37-8.46 (m, 1H);

¹³C NMR (126 MHz, CD₃OD) (as a mixture of rotamers) δ 24.2, 25.7, 30.5, 30.7, 43.5, 47.1, 47.1, 50.9, 54.4, 55.0, 98.1, 120.0, 120.7, 125.2, 127.1, 128.9, 131.1, 131.3, 147.9, 148.3, 153.3, 154.5, 157.7, 160.5, 167.9.

HR-MS (m/z): Calculated for C₂₅H₂₄N₆O₂ [M+H]⁺: 441.2039; Found: [M+H]⁺: 441.2030

### (R)-1-(3-(4-amino-3-(4-phenoxyphenyl)-1H-pyrazolo[3,4-d]pyrimidin-1-yl)piperidin-1-yl)-2-chloroethan-1-one (3a)

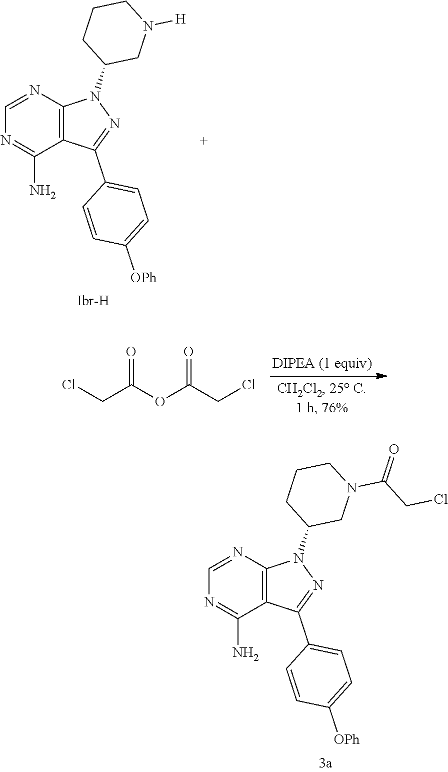 [SMILES: Nc1ncnc2c1c(-c1ccc(Oc3ccccc3)cc1)nn2[C@@H]1CCCNC1 ; O=C(CCl)OC(=O)CCl ; Nc1ncnc2c1c(-c1ccc(Oc3ccccc3)cc1)nn2[C@@H]1CCCN(C(=O)CCl)C1]

To a stirred solution of Ibr-H (387 mg, 1 mmol) in anhydrous DCM (6 mL), DIPEA (178 μL, 1 mmol) and chloroacetic anhydride (170 mg, 1 mmol) were added at 25° C. The reaction mixture was stirred at room temperature for 1 h. After completion of the reaction (as monitored by LC-MS), CH₂Cl₂ was concentrated in vacuo and the crude carboxylic acid was purified using combi flash column chromatography using MeOH:EtOAc (2:8) as eluent to give 3a in 353 mg (yield=76%).

¹H NMR (500 MHz, CD3OD) (as a mixture of rotamers) δ 1.68-1.74 (m, 0.5H), 1.79-1.89 (m, 0.5H), 1.92-2.03 (m, 0.5H), 2.04-2.14 (m, 0.5H), 2.16-2.27 (m, 1H), 2.28-2.43 (m, 1H), 3.11-3.16 (m, 0.5H), 3.46-3.50 (m, 0.5H), 3.84-3.99 (m, 1H), 4.07-4.16 (m, 0.5H), 4.21-4.40 (m, 2H), 4.55 (d, J=16.4 Hz, 0.5H), 4.78-4.84 (m, 0.5H), 4.94-4.98 (m, 1H), 7.10 (d, J=8.0 Hz, 2H), 7.13-7.23 (m, 3H), 7.36-7.46 (m, 2H), 7.63-7.73 (m, 2H), 8.27 (d, J=13.3 Hz, 1H):

¹³C NMR (125 MHz, CD3OD) (as a mixture of rotamers) δ 24.6, 25.8, 31.0, 42.4, 43.8, 47.4, 51.2, 51.9, 53.8, 54.3, 99.3, 119.3, 120.1, 120.7, 125.2, 128.9, 131.2, 131.4, 131.4, 146.3, 155.1, 155.2, 156.2, 156.5, 158.1, 160.1, 168.0.

HR-MS (m/z): calculated for C₂₄H₂₄ClN₆O₂[M+H]+: 463.1649; found: [M+H]+: 463.1645.

### (R)-1-(3-(4-amino-3-(4-phenoxyphenyl)-1H-pyrazolo[3,4-d]pyrimidin-1-yl)piperidin-1-yl)-2-hydroxyethan-1-one (pre-3)

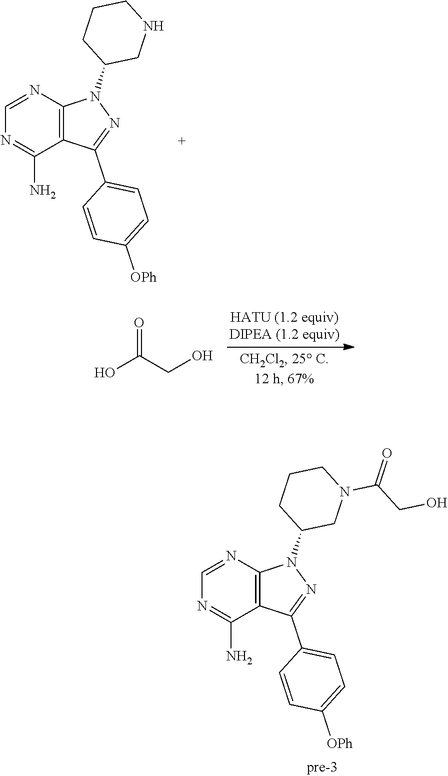

To a stirred solution of hydroxyl acetic acid (225 mg, 3 mmol) in anhydrous DCM (6 mL), HATU (1368 mg, 3.6 mmol) and DIPEA (615 μL, 3.6 mmol) and Ibr-H (1161 mg, 3 mmol) were added at 25° C. The reaction mixture was stirred at room temperature for 12 h. After completion of the reaction (as monitored by LC-MS), the reaction mixture was evaporated under vacuo and the crude product was purified using combi flash column chromatography with MeOH:EtOAc (2:8) as eluent to give Pre-3 was colorless solid in 894 mg (yield=67%).

¹H NMR (500 MHz, CD3OD) δ: 1.47-1.70 (m, 1H), 1.89-1.97 (m, 1H), 2.12 (br. s., 1H), 2.15-2.32 (m, 1H), 3.10 (t, J=11.4 Hz, 1H), 3.35-3.45 (m, 1H), 3.52-3.66 (m, 1H), 4.08-4.30 (m, 2H), 4.42 (d, J=10.3 Hz, 1H), 4.79 (br. s., 1H), 5.20 (br. s., 1H), 6.90-7.14 (m, 5H), 7.27 (t, J=7.7 Hz, 2H), 7.54 (d, J=7.8 Hz, 2H), 8.28 (d, J=19.8 Hz, 1H)

¹³C NMR (126 MHz, CD3OD) δ: 24.4, 25.3, 30.8, 43.4, 45.3, 47.1, 54.4, 54.8, 61.3, 61.5, 98.2, 120.1, 120.7, 125.4, 127.3, 131.2, 131.3, 148.3, 153.3, 154.6, 157.7, 160.5, 172.5, 172.6.

ESI-MS (m/z): calculated for C₂₄H₂₅N₆O₃ [M+H]+: 445.19; found: [M+H]+: 445.16.

### (R)-2-(3-(4-amino-3-(4-phenoxyphenyl)-1H-pyrazolo[3,4-d]pyrimidin-1-yl)piperidin-1-yl)-2-oxoethyl methanesulfonate (3b)

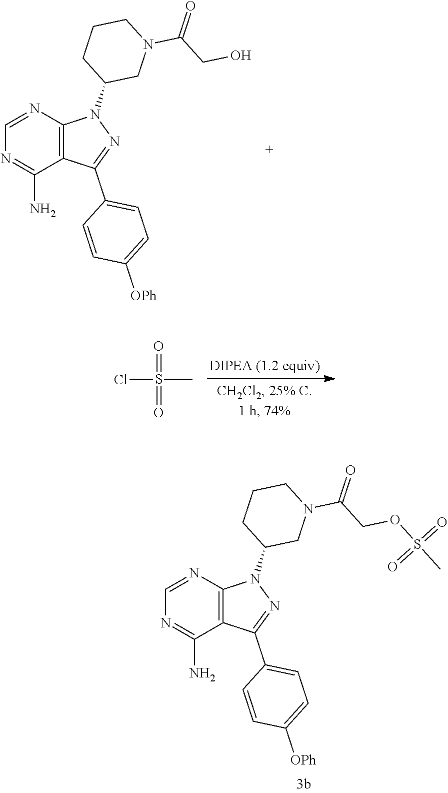

To a stirred solution of Pre-3 (23 mg, 0.05 mmol) in CH₂Cl₂ (1 mL), methane sulfonyl chloride (4.6 μL, 0.06 mmol, d=1.48), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 3b in 19.3 mg (74% yield).

¹H NMR (400 MHz, CD3OD) (as a mixture of rotamers): δ 1.67-1.90 (m, 1H), 1.98-2.20 (m, 1H), 2.21-2.32 (m, 1H), 2.36-2.49 (m, 1H), 3.12-3.23 (m, 3H), 3.35-3.43 (m, 1H), 3.63 (dd, J=12.8, 9.5 Hz, 0.7H), 3.76 (d, J=13.6 Hz, 0.7H), 3.84-3.93 (m, 0.5H), 3.94-4.01 (m, 0.5H), 4.07 (d, J=13.4 Hz, 0.5H), 4.46 (dd, J=12.8, 3.3 Hz, 0.5H), 4.96 (br. s., 1H), 4.99-5.15 (m, 2H), 7.13 (d, J=8.1 Hz, 2H), 7.16-7.25 (m, 3H), 7.46 (s, 2H), 7.71 (d, J=8.4 Hz, 2H), 8.38-8.44 (m, 1H)

¹³C NMR (101 MHz, CD3OD) δ: 24.0, 25.3, 30.7, 38.5, 43.6, 46.1, 47.2, 54.2, 54.6, 67.6, 98.5, 120.2, 120.8, 125.4, 127.6, 131.3, 131.5, 148.3, 149.4, 150.0, 153.6, 157.9, 160.7, 166.8.

HR-MS (m/z): calculated for C₂₅H₂₇N₆O₅S [M+H]+: 523.1764; found: [M+H]+: 523.1762.

### (R)-2-(3-(4-amino-3-(4-phenoxyphenyl)-1H-pyrazolo[3,4-d]pyrimidin-1-yl)piperidin-1-yl)-2-oxoethyl methylsulfamate (3c)

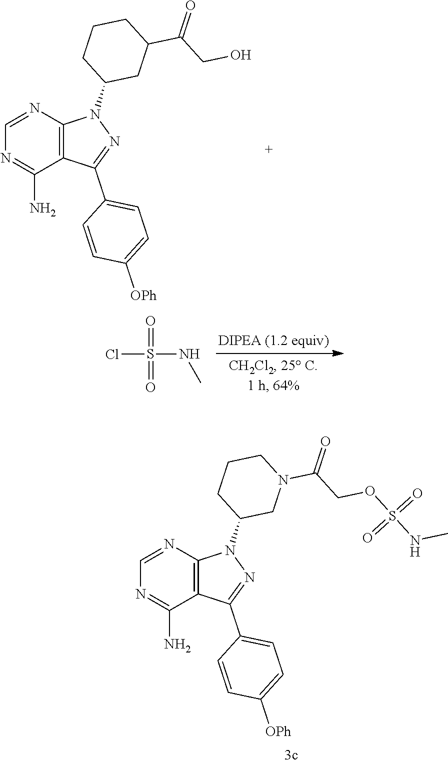

To a stirred solution of Pre-3 (230 mg, 0.5 mmol) in CH₂Cl₂ (1 mL), N-methylsulfamoyl chloride (77.4 mg, 0.6 mmol), and DIPEA (102 μL, 0.6 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 3c in 171 mg (64% yield).

¹H NMR (400 MHz, CD3OD) (as a mixture of rotamers) δ 1.73-1.83 (d, J=10.1 Hz, 1H), 2.02-2.19 (m, 1H), 2.22-2.32 (m, 1H), 2.40 (br. s., 1H), 2.68 (s, 1.2H), 2.74 (s, 1.6H), 3.39 (d, J=12.5 Hz, 0.5H), 3.61 (dd, J=12.2, 10.0 Hz, 0.7H), 3.76-3.94 (m, 1H), 3.97-4.13 (m, 1H), 4.48 (dd, J=12.8, 3.3 Hz, 0.7H), 4.77-4.84 (m, 0.4H), 4.95 (dd, J=9.4, 4.7 Hz, 0.7H), 5.04 (td, J=8.3, 4.2 Hz, 0.5H), 7.13 (d, J=7.9 Hz, 2H), 7.16-7.24 (m, 3H), 7.40-7.47 (m, 2H), 7.71 (d, J=8.4 Hz, 2H), 8.40 (s, 1H):

¹³C NMR (101 MHz, CD3OD) δ: 24.1, 25.4, 29.7, 30.6, 30.7, 43.6, 46.4, 47.2, 54.2, 54.6, 67.4, 67.6, 98.8, 120.2, 120.8, 125.4, 127.7, 127.7, 131.3, 131.5, 149.9, 150.0, 150.6, 153.7, 157.9, 160.7, 166.8, 167.0.

HR-MS (m/z): calculated for C₂₅H₂₈N₇O₅S [M+H]+: 538.1873; found: [M+H]+: 538.1875.

### (R)-2-(3-(4-amino-3-(4-phenoxyphenyl)-1H-pyrazolo[3,4-d]pyrimidin-1-yl)piperidin-1-yl)-2-oxoethyl benzylsulfamate (3d)

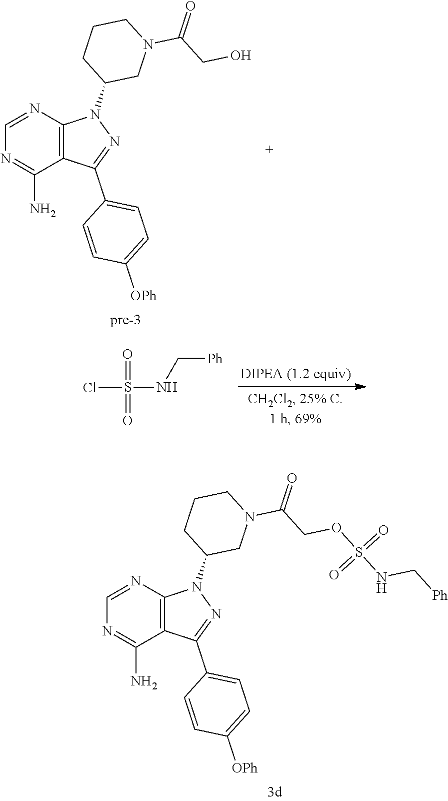 [SMILES: Nc1ncnc2c1c(-c1ccc(Oc3ccccc3)cc1)nn2[C@@H]1CCCN(C(=O)CO)C1 ; O=S(=O)(Cl)NCc1ccccc1 ; Nc1ncnc2c1c(-c1ccc(Oc3ccccc3)cc1)nn2[C@@H]1CCCN(C(=O)COS(=O)(=O)NCc2ccccc2)C1]

To a stirred solution of Pre-3 (23 mg, 0.05 mmol) in CH₂Cl₂ (1 mL), benzylsulfamoyl chloride (12.3 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 3d in 21.1 mg (69% yield).

¹H NMR (400 MHz, CD3OD) δ: 1.75 (dd, J=17.2, 3.7 Hz, 1H), 1.98-2.17 (m, 1H), 2.18-2.32 (m, 1H), 2.32-2.46 (m, 1H), 3.35-3.43 (m, 0.5H), 3.60 (dd, J=12.8, 9.5 Hz, 0.5H), 3.69 (d, J=13.9 Hz, 0.5H), 3.85 (dd, J=13.6, 8.1 Hz, 0.5H), 3.94 (dd, J=13.8, 3.6 Hz, 0.5H), 4.04 (d, J=13.2 Hz, 0.5H), 4.19 (s, 1H), 4.27 (s, 1H), 4.44 (dd, J=12.9, 3.2 Hz, 0.6H), 4.60-4.60 (m, 0.5H) 4.67-4.79 (m, 2H), 4.92-4.98 (m, 0.7H), 4.99-5.07 (m, 0.5H), 7.12 (d, J=7.9 Hz, 2H), 7.14-7.23 (m, 3H), 7.31 (d, J=4.2 Hz, 2H), 7.37 (d, J=4.6 Hz, 2H), 7.40-7.47 (m, 2H), 7.65-7.76 (m, 2H), 8.40 (d, J=12.1 Hz, 1H):

¹³C NMR (101 MHz, CD3OD) δ: 24.0, 25.3, 30.6, 30.7, 43.6, 46.3, 47.2, 50.0, 54.2, 54.6, 67.3, 67.6, 98.8, 120.2, 120.8, 125.4, 127.5, 129.0, 129.2, 129.4, 129.8, 131.3, 131.5, 138.8, 149.3, 150.1, 153.6, 155.3, 157.9, 160.7, 166.8.

HR-MS (m/z): calculated for C₃₁H₃₂N₇O₅S [M+H]+: 614.2186; found: [M+H]+: 614.2188.

### (R)-2-(3-(4-amino-3-(4-phenoxyphenyl)-1H-pyrazolo[3,4-d]pyrimidin-1-yl)piperidin-1-yl)-2-oxoethyl phenylsulfamate (3e)

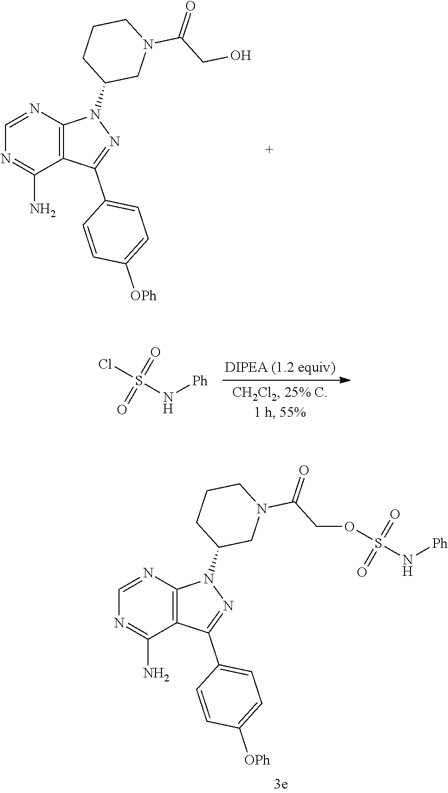 [SMILES: Nc1ncnc2c1c(-c1ccc(Oc3ccccc3)cc1)nn2[C@@H]1CCCN(C(=O)CO)C1 ; O=S(=O)(Cl)Nc1ccccc1 ; Nc1ncnc2c1c(-c1ccc(Oc3ccccc3)cc1)nn2[C@@H]1CCCN(C(=O)COS(=O)(=O)Nc2ccccc2)C1]

To a stirred solution of Pre-3 (23 mg, 0.05 mmol) in CH₂Cl₂ (1 mL), phenylsulfamoyl chloride (11.5 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 3e in 16.4 mg (55% yield).

¹H NMR (500 MHz, CD3OD) (as a mixture of rotamers) δ: 1.60-1.72 (m, 1H), 1.96-2.07 (m, 1H), 2.15-2.25 (m, 1H), 2.29-2.40 (m, 1H), 3.21-3.28 (m, 1H), 3.48 (d, J=13.6 Hz, 1H), 3.73-3.81 (m, 1H), 3.89 (d, J=9.9 Hz, 0.6H), 4.02 (d, J=13.1 Hz, 0.6H), 4.40 (d, J=12.5 Hz, 0.6H), 4.73-4.84 (m, 2H), 7.02 (t, J=7.2 Hz, 1H), 7.09 (d, J=8.0 Hz, 1H), 7.13-7.24 (m, 6H), 7.26 (d, J=8.0 Hz, 1H), 7.35 (t, J=7.8 Hz, 1H), 7.44 (t, J=7.1 Hz, 2H), 7.65-7.74 (m, 2H), 8.37 (d, J=8.0 Hz, 1H).

HR-MS (m/z): calculated for C₃₀H₃₀N₇O₅S [M+H]+: 600.2029; found: [M+H]+: 600.2032.

### Synthesis of (R)-1-(3-(4-amino-3-(4-phenoxyphenyl)-1H-pyrazolo[3,4-d]pyrimidin-1-yl)piperidin-1-yl)-2-(methylsulfonyl)ethan-1-one (3f)

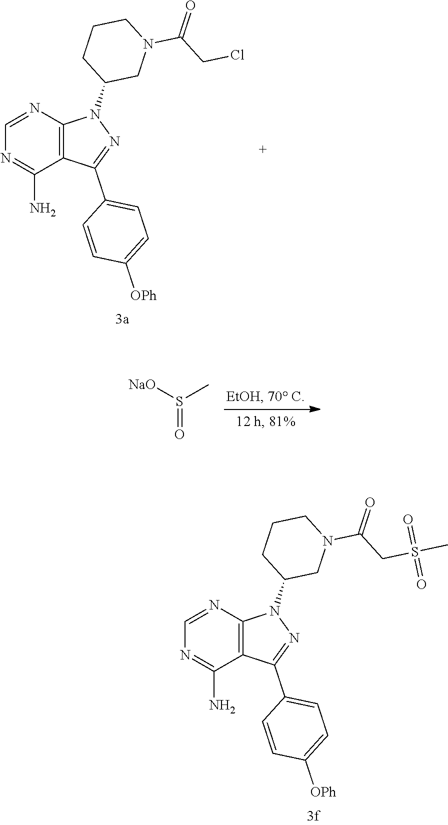

To a stirred solution of 3a (23 mg, 0.05 mmol) in ethanol (1 mL), sodium methanesulfinate (10.2 mg, 0.1 mmol) was added at 25° C. The reaction mixture was stirred at room temperature for 12 h at 70° C. After completion of the reaction (as monitored by LC-MS), ethanol was concentrated in vacuo and the crude product was purified by preparative HPLC using water:ACN (0.1% formic acid) solvent gradient to afford white solid 3f in 20.4 mg (81% yield).

¹H NMR (400 MHz, CD3OD) (as a mixture of rotamers) δ: 1.71-1.77 (m, 0.5H), 1.83-1.94 (m, 0.5H), 1.97-2.03 (m, 0.5H), 2.06-2.15 (m, 0.5H), 2.21-2.31 (m, 1H), 2.32-2.44 (m, 1H), 3.12 (d, J=2.6 Hz, 3H), 3.17-3.24 (m, 0.5H), 3.38-3.47 (m, 0.5H), 3.58 (dd, J=12.7, 10.0 Hz, 0.6H), 3.95-4.09 (m, 1H), 4.23-4.38 (m, 0.5H), 4.32 (d, J=14.5 Hz, 1H), 4.39-4.52 (m, 1.5H), 4.60 (dd, J=12.8, 3.5 Hz, 0.6H), 4.91-4.98 (m, 0.6H), 5.06-5.12 (m, 1H), 7.13 (d, J=8.1 Hz, 2H), 7.16-7.24 (m, 3H), 7.36-7.47 (m, 2H), 7.71 (dd, J=8.6, 4.2 Hz, 2H), 8.41 (d, J=3.7 Hz, 1H).

¹³C NMR (101 MHz, CD3OD) (as a mixture of rotamers) δ: 24.4, 25.5, 31.0, 42.3, 43.7, 47.2, 51.7, 54.4, 55.0, 58.3, 98.5, 120.2, 120.8, 125.4, 127.6, 131.3, 131.5, 137.5 (m) 148.3, 149.5, 149.7, 153.6, 153.8, 157.9, 160.7, 163.5.

ESI-MS (m/z): calculated for C₂₅H₂₇N₆O₄S [M+H]+: 507.18; found: [M+H]+: 507.19.

### Synthesis of (R)-1-(3-(4-amino-3-(4-phenoxyphenyl)-1H-pyrazolo[3,4-d]pyrimidin-1-yl)piperidin-1-yl)-2-(phenylsulfonyl)ethan-1-one (3g)

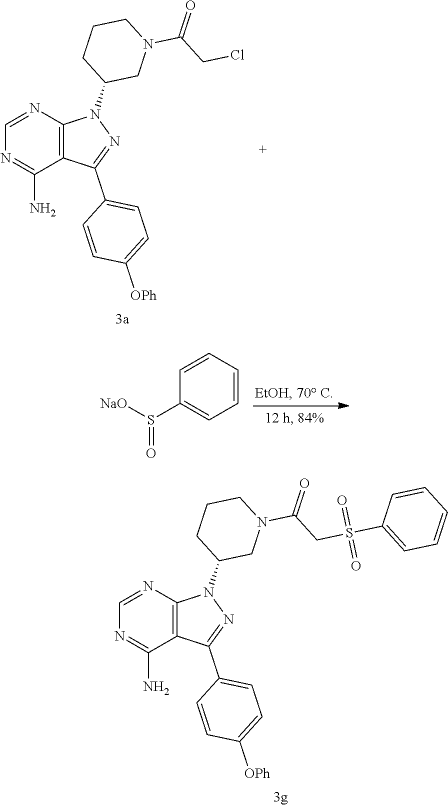

To a stirred solution of 3a (23 mg, 0.05 mmol) in ethanol (1 mL), sodium phenylsulfinate (10.2 mg, 0.1 mmol) was added at 25° C. The reaction mixture was stirred at room temperature for 12 h at 70° C. After completion of the reaction (as monitored by LC-MS), ethanol was concentrated in vacuo and the crude product was purified by preparative HPLC using water:ACN (0.1% formic acid) solvent gradient to afford white solid 3g in 23.8 mg (84% yield).

¹H NMR (500 MHz, CD3OD) δ: 1.61-1.72 (m, 0.5H), 1.76-1.86 (m, 0.5H), 1.93-2.01 (m, 0.5H), 2.04-2.12 (m, 0.5H), 2.19-2.29 (m, 1H), 2.29-2.41 (m, 1H), 3.09-3.14 (m, 0.5H), 3.35 (m, 0.5H), 3.50 (dd, J=12.8, 10.0 Hz, 0.5H), 4.03 (d, J=13.9 Hz, 0.6H), 4.19-4.29 (m, 1H), 4.37-4.40 (m, 0.5H), 4.51 (d, J=14.3 Hz, 1H), 4.58-4.68 (m, 1H), 4.80-4.81 (m, 0.5H), 5.05-5.15 (m, 0.5H), 7.09-7.15 (m, 2H), 7.15-7.24 (m, 3H), 7.40-7.47 (m, 2H), 7.60-7.68 (m, 2H), 7.71 (d, J=8.5 Hz, 2H), 7.73-7.79 (m, 1H), 7.93 (d, J=8.4 Hz, 1H), 7.98 (d, J=8.5 Hz, 1H), 8.43 (d, J=10.7 Hz, 1H):

¹³C NMR (126 MHz, CD3OD) δ: 24.4, 25.5, 30.7, 31.0, 43.8, 47.3, 51.8, 54.5, 55.1, 60.3, 98.4, 98.5, 120.2, 120.9, 125.4, 127.4, 129.6, 130.6, 131.3, 131.4, 131.5, 135.6, 140.7, 140.9, 148.4, 148.5, 148.8, 149.0, 153.5, 153.7, 154.9, 155.1, 157.9, 160.8, 162.7, 162.8.

ESI-MS (m/z): calculated for C₃₀H₂₉N₆O₄S [M+H]+: 569.20; found: [M+H]+: 569.29.

### Synthesis of 3 h

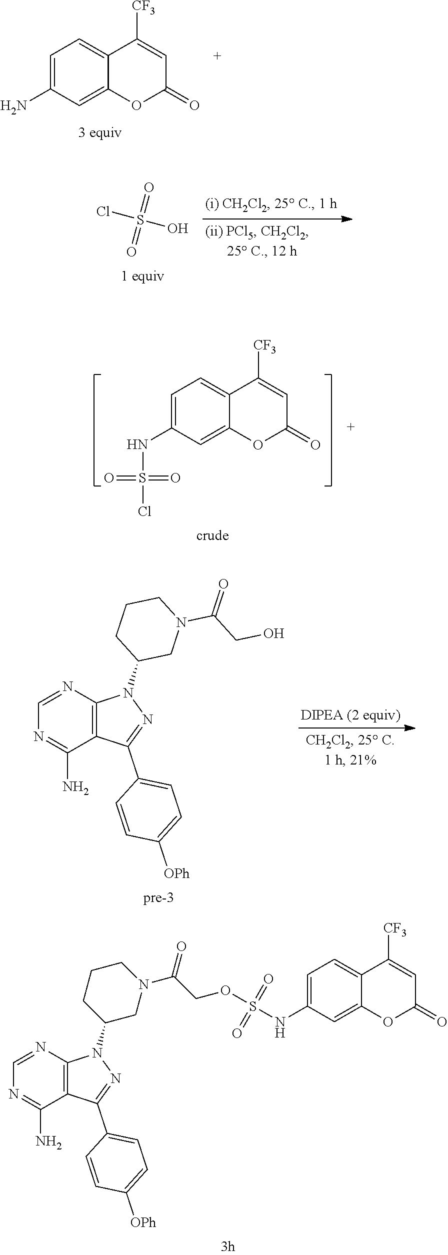

To a stirred solution of amine (69 mg, 0.3 mmol) in CH₂Cl₂ (1 mL), chloro methane sulfonyl chloride (7.7 μL, 0.1 mmol,) was added at 0° C. The reaction mixture was stirred at room temperature for 1 h. After completion of the reaction organic layer was concentrated in vacuo. The crude product was dissolved CH₂Cl₂ (1 mL) and PCl₅ (20 mg, 0.1 mmol) was added at 0° C. The reaction mixture was stirred at room temperature for 2 h. After completion of the reaction (as monitored by LC-MS), the reaction mixture is filtered and washed with dichloromethane. The filtrate was concentrated and used as such for the next reaction.

To a stirred solution of Pre-3 (23 mg, 0.05 mmol) in CH₂Cl₂ (1 mL), 7-amino, 4-trifluoro coumarine sulfamoyl chloride (0.1 mmol), and DIPEA (17.5 μL, 0.1 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 3 h in 7.5 mg (21% yield).

HR-MS (m/z): calculated for C₃₄H₂₉F₃N₇O₇S [M+H]+: 736.1801; found: [M+H]+: 736.1802.

### Synthesis of methyl (R)-3-(4-amino-3-(4-phenoxyphenyl)-1H-pyrazolo[3,4-d]pyrimidin-1-yl)piperidine-1-sulfonate (3i)

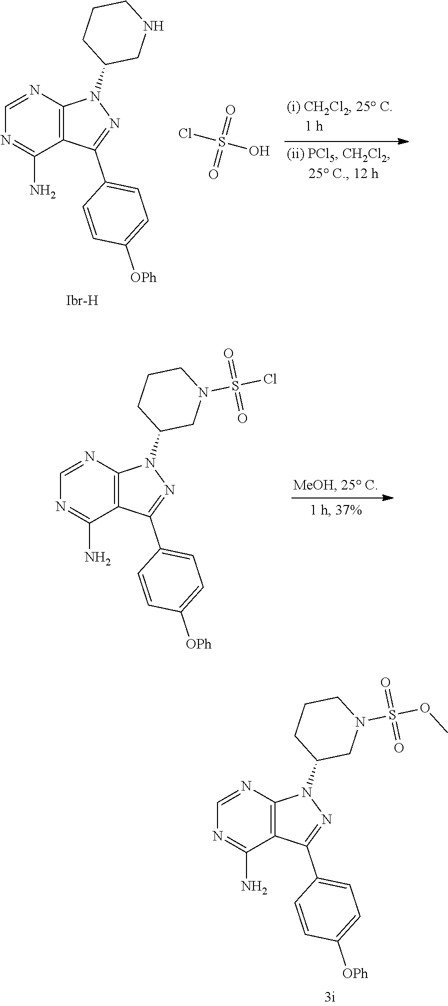 [SMILES: Nc1ncnc2c1c(-c1ccc(Oc3ccccc3)cc1)nn2[C@@H]1CCCNC1 ; O=S(=O)(O)Cl ; Nc1ncnc2c1c(-c1ccc(Oc3ccccc3)cc1)nn2[C@@H]1CCCN(S(=O)(=O)Cl)C1 ; COS(=O)(=O)N1CCC[C@@H](n2nc(-c3ccc(Oc4ccccc4)cc3)c3c(N)ncnc32)C1]

To a stirred solution of amine (116 mg, 0.3 mmol) in CH₂Cl₂ (1 mL), chloro methane sulfonyl chloride (7.7 μL, 0.1 mmol,) was added at 0° C. The reaction mixture was stirred at room temperature for 1 h. After completion of the reaction organic layer was concentrated in vacuo. The crude product was dissolved CH₂Cl₂ (1 mL) and PCl₅ (20 mg, 0.1 mmol) was added at 0° C. The reaction mixture was stirred at room temperature for 2 h. After completion of the reaction (as monitored by LC-MS), the reaction mixture is filtered and washed with dichloromethane. The filtrate was concentrated and used as such for the next reaction. ⅓^{rd} of the crude product was dissolved in MeOH and allowed it to stir for 1 hour. After completion of the reaction (as monitored by LC-MS), the reaction mixture is concentrated in vacuo and the crude product was purified by preparative HPLC using water:ACN (0.1% formic acid) solvent gradient to afford white solid 3i in 7.5 mg (21% yield).

¹H NMR (500 MHz, CD₃OD) δ: 1.91 (q, J=12.7 Hz, 1H), 2.02-2.10 (m, 1H), 2.19-2.26 (m, 1H), 2.26-2.33 (m, 1H), 3.07 (t, J=12.2 Hz, 1H), 3.52 (t, J=11.3 Hz, 1H), 3.81 (d, J=12.7 Hz, 1H), 3.88 (s, 3H), 3.97 (d, J=11.7 Hz, 1H), 5.07 (br. s., 1H), 7.13 (d, J=7.7 Hz, 2H), 7.16-7.27 (m, 4H), 7.44 (t, J=7.3 Hz, 2H), 7.71 (d, J=7.3 Hz, 2H), 8.40 (s, 1H):

¹³C NMR (126 MHz, CD₃OD) δ: 23.5, 28.8, 46.4, 49.7, 52.9, 56.1, 97.2, 118.6, 119.3, 123.9, 126.2, 129.7, 129.9, 146.4, 149.4, 152.5, 156.4, 159.1.

HR-MS (m/z): calculated for C₂₃H₂₅N₆O₄S [M+H]+: 481.1658; found: [M+H]+: 481.1655.

### Synthesis of prop-2-yn-1-yl (R)-3-(4-amino-3-(4-phenoxyphenyl)-1H-pyrazolo[3,4-d]pyrimidin-1-yl)piperidine-1-sulfonate (3j)

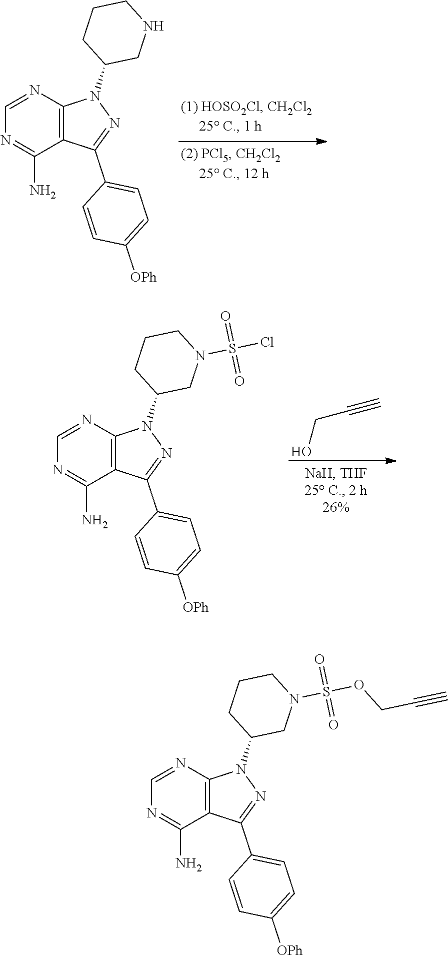

The synthesized Ibrutinib sulfamoyl chloride (⅓^{rd} of the crude from the previous step, 0.1 mmol) was dissolved in THF (0.3 mL) followed by the addition of propargyl alcohol (27.2 mg, 10 mmol) and sodium hydride (10 mmol, 40 mg) at 0° C. The reaction mixture was stirred at room temperature for 2 h. After completion of the reaction (as monitored by LC-MS), the reaction mixture is filtered and washed with dichloromethane. The filtrate was concentrated and purified by preparative HPLC using water:ACN (0.1% formic acid) solvent gradient to afford white solid 3j in 13.1 mg (6% yield).

¹H NMR (500 MHz, CD₃OD) δ: 1.84-1.97 (m, 1H), 2.03-2.10 (m, 1H), 2.21 (dd, J=12.7, 3.2 Hz, 1H), 2.31 (qd, J=12.2, 3.8 Hz, 1H), 3.10 (td, J=12.2, 2.5 Hz, 1H), 3.23 (t, J=2.3 Hz, 1H), 3.56 (t, J=11.3 Hz, 1H), 3.81 (d, J=12.7 Hz, 1H), 3.97 (dd, J=11.9, 3.9 Hz, 1H), 4.83 (d, J=2.3 Hz, 2H), 5.00-5.09 (m, 1H), 7.12 (m, J=8.1 Hz, 2H), 7.15-7.24 (m, 3H), 7.43 (t, J=7.8 Hz, 2H), 7.70 (m, J=8.5 Hz, 2H), 8.38 (s, 1H):

¹³C NMR (125 MHz, CD₃OD) δ: 25.0, 30.3, 47.9, 51.2, 54.3, 59.3, 79.0, 97.4, 120.2, 120.8, 125.4, 127.3, 131.3, 131.5, 151.7, 155.9, 166.9.

HR-MS (m/z): calculated for C₂₅H₂₅N₆O₄S [M+H]+: 505.1658; found: [M+H]+: 481.1652.

### Example 2

### Sulfamate Acetamides as Covalent Inhibitors

To show that sulfamates can work as electrophilic warheads in targeted covalent inhibitors, Ibrutinib was chosen, an acrylamide-based covalent inhibitor for Bruton's tyrosine kinase (BTK), and replaced its acrylamide electrophile with sulfamate acetamides. Ibrutinib is an FDA-approved drug for B-cell malignancies and inhibits BTK phosphorylation by forming an irreversible bond at Cys481. The synthesis of Ibrutinib-based sulfamate acetamides (FIG. 2A; 3c-3e) is provided in Example 1. In addition to sulfamates, chloro-(3a), sulfonate-(3b) and sulfone (3f and 3g) acetamides analogs of Ibrutinib were synthesized.

To assess their covalent labeling efficiency, an intact protein mass spectrometry experiments with recombinant BTK (2 μM) and compounds 3a-3g (2 μM; buffer: 20 mM tris, 50 mM NaCl, pH 8; 25° C.) was conducted. All three sulfamate- (3c-3e) and sulfonate-(3b) acetamides labeled BTK by more than 95% within 10 minutes with the elimination of sulfamic acid or sulfonic acid leaving groups (FIG. 2B, 2C). The labeling efficiency of these compounds is similar to Ibrutinib and the chloroacetamide 3a. The two sulfone acetamides (3f and 3g) failed to react covalently with BTK under the reaction conditions.

To understand the potential of these compounds as BTK inhibitors, in vitro kinase activity assays were conducted for all Ibrutinib derivatives against BTK (FIG. 2D). The alkyl sulfamate compounds (3c and 3d) showed similar IC₅₀ to Ibrutinib (around 10 nM; FIGS. 2D, 2E) whereas the phenyl sulfamate (3e) showed a 10-fold weaker IC₅₀ (100 nM). 3a and 3b also showed potent BTK inhibition. The presumably non-covalent sulfone compound (3g) showed poor inhibition of BTK with IC₅₀=0.5 μM.

To understand the importance of covalent bond formation and off-target selectivity, the same assay was conducted with ibrutinib, 3a, 3c, 3d, and 3e against BTK C₄₈₁S mutant and EGFR, a therapeutically relevant off-target of ibrutinib. Compounds 3a, 3c and 3d lost 30-85 fold potency against the C₄₈₁S mutant (Table 1), indicating their dependence on covalent bond formation. While the chloroacetamide 3a showed only ˜10-fold selectivity against EGFR, the sulfamates showed 20-30 fold selectivity as presented in Table 1:

TABLE 1
In vitro kinase activity assay of Ibrutinib analogs. Activity assay was performed
with 0.5 nM BTK, and 5 μM ATP with all the Ibr-sulfamates (3a-3g). For compounds
Ibrutinib, 3a, 3c, 3d, and 3e, kinase assay was conducted with BTK C481S mutant and EGFR
also. The IC₅₀ values and the ratios of off-target/BTK are given in the table.

| Compound | BTK (IC₅₀ μM) | C481S (IC₅₀ μM) | EGFR (IC₅₀ μM) | C481S/BTK | EGFR/BTK |
|---|---|---|---|---|---|
| Ibrutinib | 0.012 | 2.000 | 1.670 | 160.0 | 133.6 |
| 3a | 0.029 | 0.795 | 0.261 | 27.0 | 8.9 |
| 3b | 0.046 | — | — | — | — |
| 3c | 0.0086 | 0.740 | 0.241 | 85.6 | 27.9 |
| 3d | 0.024 | 1.280 | 0.533 | 52.8 | 22.0 |
| 3e | 0.095 | 0.275 | 1.960 | 2.9 | 20.6 |
| 3g | 0.024 | — | — | — | — |

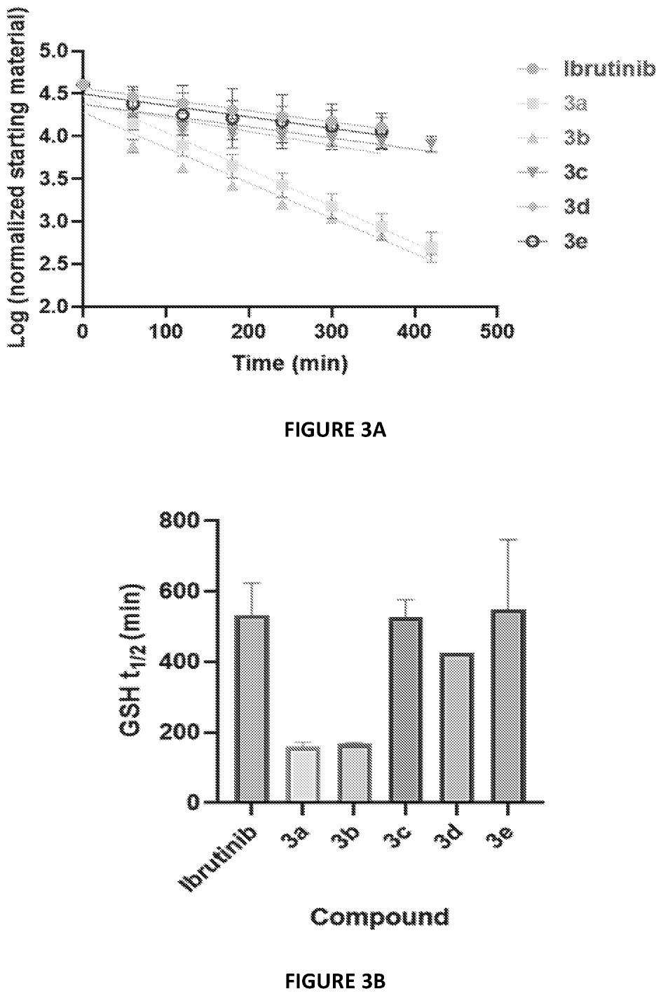

To assess the thiol reactivity of these analogs, a GSH consumption assay was performed and found that these compounds follow a similar reactivity pattern to the model compounds (1a-1 h). The sulfamate compounds (3c-3e) showed GSH half-lives (t_{1/2}˜8 h) similar to Ibrutinib (FIG. 2E and FIG. 3A). On the other hand, 3a and 3b showed a 4-fold higher reactivity than Ibr-sulfamates (t_{1/2}=2 h; FIG. 2E and FIG. 3B). When correlating the reactivity of these compounds with their kinase activities, we found that the sulfamate compounds 3c and 3d are potent inhibitors with relatively low thiol reactivity. Further, we have also found that these sulfamate electrophiles show high buffer stability (<5% hydrolysis) compared to chloro- (25% hydrolysis) and sulfonate (75% hydrolysis) electrophiles (37° C.; 4 days). Moreover, the sulfamate analogs displayed improved metabolic stability when incubated with human liver microsomes (FIGS. 4A-4C). In particular, over 30% of methyl sulfamate (3c) remained intact after a five-minute incubation whereas Ibrutinib was completely degraded (<5%).

### Example 3

Sulfamate Acetamides are Compatible with Cells and In Vivo Administration

To assess the cellular efficacy of these compounds, the inhibition of BTK autophosphorylation in Mino cells was followed. After one hour pre-incubation with the inhibitors and B-cell activation with an anti-IgM antibody, BTK autophosphorylation was followed by Western blot. All of the compounds completely inhibited phosphorylation at 100 nM except 3g. The dose-dependent treatment of compounds 3c (IC₅₀=3.6 nM) and 3b (IC₅₀=6 nM) showed excellent inhibition, similar to Ibrutinib (IC₅₀=2.1 nM; FIG. 5A). The structurally similar and more reactive analog 3a showed 20-fold less potency in cellular pBTK inhibition (IC₅₀=86 nM) potentially due to reaction with off-targets.

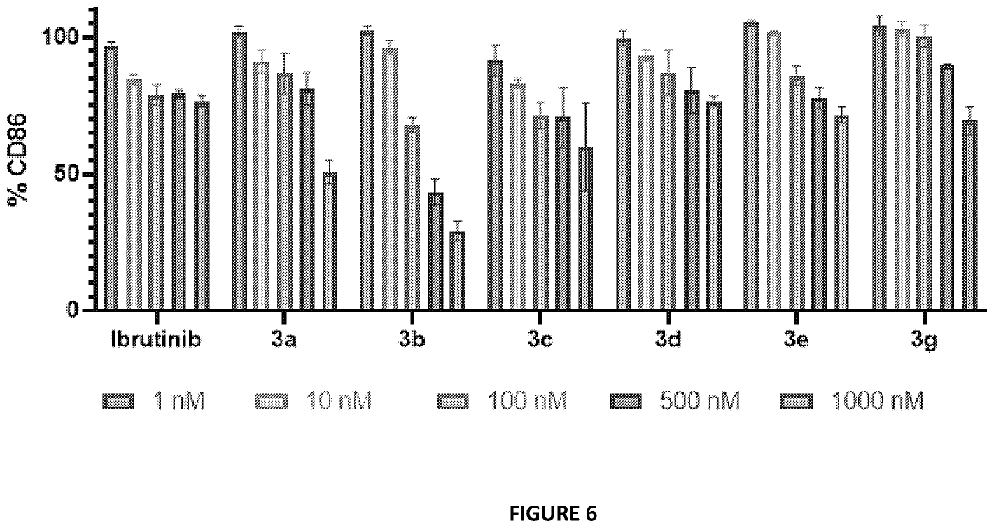

B-cell receptor signaling inhibition in primary mouse B-cells by Ibrutinib was evaluated as well as four of its analogs. Mouse splenocytes were incubated (24 h; 37° C.) with the inhibitors at various concentrations and treated with anti-IgM. To examine the effect specifically on B-cells, B220+ cells were gated and assessed activation by flow cytometry detection of CD86 expression. All five inhibitors with sulfamate acetamides and sulfonates (3a-3e) showed similar inhibition in B-cell activation to Ibrutinib (FIG. 5B & FIG. 6).

To assess the efficiency of these new inhibitors in a clinically relevant model, we tested the potency of the three sulfamate analogs (3c-3e) along with Ibrutinib and 3a in chronic lymphocytic leukemia (CLL) primary patient samples for the inhibition of BTK phosphorylation and its downstream pathway targets pPLCγ2, pAkt and pERK⁴¹. CLL cells (20×10⁶/mL) were incubated with DMSO, Ibrutinib, or Ibrutinib-based sulfamates (3c-3e) at the indicated doses at 37° C. After 2 hours of incubation, the cells were stimulated for 15 minutes or left untreated. Cell lysates were extracted and analyzed by Western blot. All of the compounds inhibited p-BTK (>80%) at 100 and 1000 nM concentrations (FIG. 5C). Downstream targets p-PLCγ2, p-Akt and p-ERK were also dose-dependently downregulated. Compared to Ibrutinib, 3c and 3d, as well as 3a showed similar p-ERK and p-Akt inhibition at 1 μM (FIG. 5C).

Taken together these three cellular experiments indicate that sulfamate acetamides showed target engagement in cells, stability to cellular conditions and comparable potency to Ibrutinib (FIG. 5A-5C).

The effect of the Ibrutinib sulfamates in vivo was evaluated. compound 3c was tested in the TCL1 adoptive transfer mouse model for CLL. In this experiment, immune-competent healthy mice received an adoptive transfer of 4×10⁷ TCL1 splenocytes, obtained from full leukemic TCL1 transgenic mice by intravenous injection (FIG. 5D). IgM+/CD5+ cells were monitored weekly and treatment (or mock treatment) was started four weeks after transplantation when CD5+ were >30% in the blood. The mice received a solution containing sulfamate 3c (0.16 mg/mL in 1% cyclodextrin water) ad libitum in the drinking water. The CD5+ cells count significantly decreased following treatment with 3c compared to untreated mice in which the cell count increased (p=0.002; FIG. 5E). Moreover, spleens were isolated from the mice after two weeks of treatment and quantified. The spleens isolated from treated mice were visually smaller than untreated mice (not shown). The spleens isolated from untreated mice were visually larger than untreated mice. To evaluate BTK engagement of the probe in vivo, the dissected spleens were extracted with RIPA buffer and incubated with an ibrutinib-alkyne analog followed by the click reaction with TAMRA-azide, and imaged via gel fluorescence (FIG. 5F). The three untreated mice showed a prominent BTK band while 3c treated mice do not show probe labeling which confirms engagement of BTK by 3c. These results suggest that the sulfamate acetamide electrophile is compatible with in vivo administration, shows oral bioavailability, sufficient exposure and in this model, a pronounced therapeutic effect.

In order to check the cellular selectivity and identify potential off-targets, isoDTB ABPP [Zanon, P. R. A. et al. Isotopically Labeled Desthiobiotin Azide (isoDTB) Tags Enable Global Profiling of the Bacterial Cysteinome. Angew. Chem. Int. Ed Engl. 2020, 59 (7) 2829-2836 which is incorporated herein by reference] experiments were performed with Ibrutinib and compound 3c (FIG. 5G). In this experiment the compounds showed similar proteomic selectivity. Only 11 and 10 off-targets for Ibrutinib and 3c respectively (H/L ratio>2) were detected. Out of which, five proteins were shared between Ibruinib and 3c. However, BTK was not identified in this experiment. In order to identify other relevant kinase off-targets, a competitive pull-down (FIG. 5H) experiment was performed in which Mino cells were first incubated with either Ibrutinib, 3c, 3d (1 μM) or DMSO control, followed by incubation with an Ibrutinib alkyne probe (1 μM). This was followed by a reaction with biotin-azide using CuAAC, enrichment of the labeled proteins, and their quantification by tryptic digestion and LC/MS/MS analysis. This experiment identified BTK as the most prominent target for all three compounds. BLK was also identified as a known off-target. Overall, with a threshold of fold-enrichment>4 and p-value<0.05 only 6, 10 and 20 off-targets were identified for 3c, Ibrutinib and 3d respectively, indicating again that the sulfamates have comparable proteomic selectivity.

### Example 4

### Sulfamate Warheads for Covalent Ligand Directed Release Chemistry

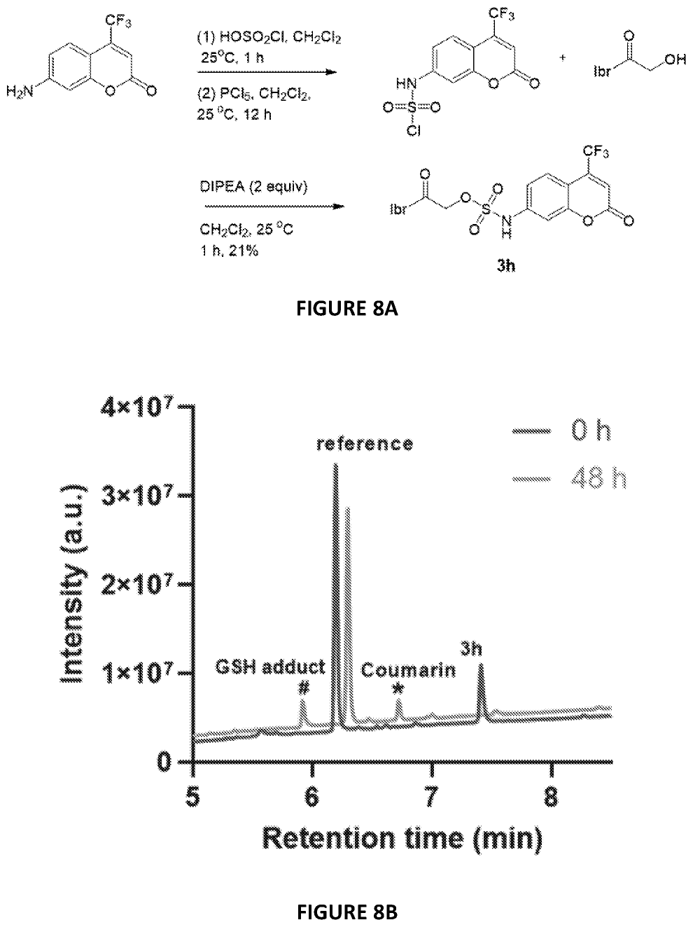

An Ibrutinib attached sulfamate containing 4-trifluoro 7-amino coumarin was synthesized (3 h; FIG. 7A, FIG. 8A). compound 3h (2 μM) was incubated with BTK (2 PM; pH 8) and measured the fluorescence over time at 435 nm. a significant increase in fluorescence (4-fold) was observed in the presence of BTK due to the release of 4-trifluoro 7-amino coumarin (FIG. 7B). Pre-incubation of BTK with Ibrutinib abrogated the increase in fluorescence, demonstrating that binding to BTK active-site and/or Cys481 are required for the release of coumarin. LC/MS measurement at the end of the fluorescence experiment showed an increase in the molecular weight of the protein correlating to the molecular weight of the compound absent the coumarin sulfamate (FIG. 7C). To identify the released coumarin derivative, compound 3h was reacted with GSH (5 mM, pH 8, 37° C., 20 mM tris buffer) and analyzed by LC/MS at 0 h and 48 h. The LC/MS spectrum clearly showed the formation of a GSH adduct and released 4-trifluoro 7-amino coumarin after 48 h (FIG. 8B).

### Example 5

### Traceless Labeling of Endogenous Proteins Using Sulfamate CoLDR

To assess irreversible labeling and validate the ligand release mechanism, sulfamate analogs with a methyl group (3i)- or an alkyne tag (3j) were synthesized (FIG. 7E).

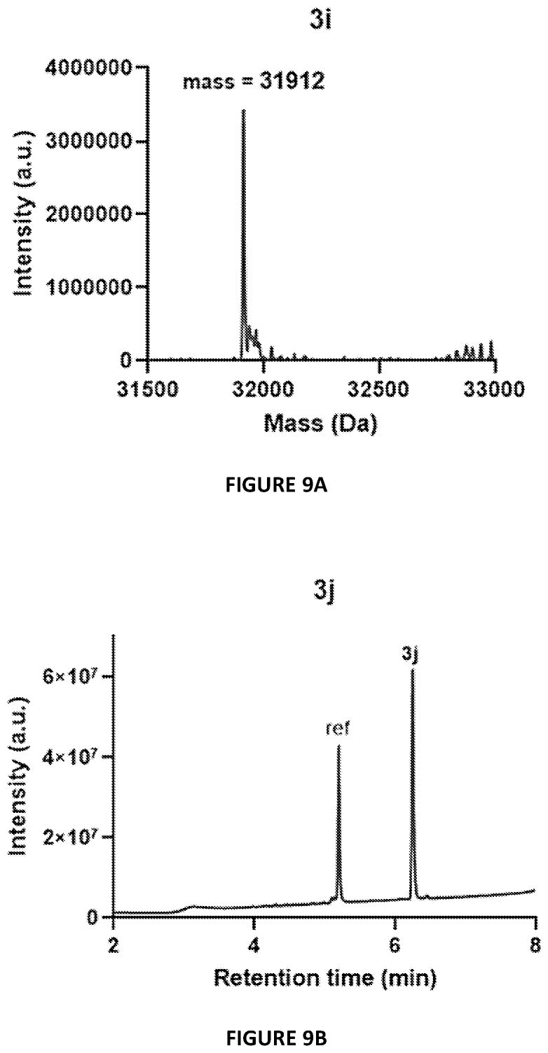

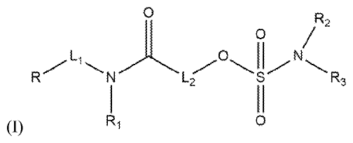

The compounds (3i, 3j) (2 μM) were incubated with recombinant BTK (2 μM) and monitored the reaction via LC/MS. The analysis of the reaction with 3j for example, verified that the shift in mass corresponds to labeling BTK with the alkyne group and release of Ibr-H (FIG. 6F). 3i installed a single methyl on BTK (FIG. 9A). We have assessed the reactivity of 3j using a GSH consumption assay along with Ibrutinib. We have found that, under the same conditions, compound 3j did not undergo any reaction with GSH indicating its low thiol reactivity (FIG. 9B).

In addition to the in vitro labeling of BTK by our probe (compounds), their engagement in cells was tested. Mino cells were incubated with probe 3j for 2 h, followed by CuAAC with TAMRA-azide to the alkyne tag in lysate. The samples were imaged via gel fluorescence. Probe 3j showed BTK labeling (70 kDa) at a concentration of 100 nM (FIG. 7G). When cells were pre-incubated with Ibrutinib, the band at 70 kDa disappeared, confirming that the probe was indeed labeling BTK. Despite its low reactivity, the sulfamate probe 3j has three apparent off-targets which were not competed by Ibrutinib. When conducting this cellular labeling experiment at higher concentrations, to assess the saturation of BTK binding, maximal labeling reached at 1 μM was observed. At 10 μM compound 3j presented much stronger fluorescence than the acrylamide alkyne probe (not shown) which may be the result of the Ibr-H moiety leaving.

In order to examine the effect of BTK modification by these probes on its cellular activity, BTK activity assays were performed. Mino cells were incubated with probe 3j followed by BTK activation using anti-human IgM. BTK autophosphorylation was followed by Western blot to assess its activity. While Ibrutinib completely abolished BTK autophosphorylation, BTK remained active after labeling with 3j. Further, to ensure that the activity did not originate from unlabeled BTK, cells were incubated with 100 nM Ibrutinib for 45 min before activation with IgM. While Ibrutinib alone completely inhibited BTK's activity, the sulfamate CoLDR probe 3j rescued this inhibition which confirms the cellular engagement of the BTK by the probe in its active form. A slight reduction in phosphorylation upon Ibrutinib incubation is observed which may indicate incomplete BTK labeling by the probe (FIG. 7H).

### Example 6

### Reactivity of Sulfamate Acetamide Compounds (Model Compounds)

To investigate the reactivity of sulfamate acetamide compounds, nine sulfo-based model electrophile compounds were synthesized (See Example 7) including two sulfonates (1b and 1c) five sulfamates (1d-1 h), and two sulfones (1i and 1j)

The model compounds are as shown in the below table:

| Compound | |
|---|---|
| number | Structure |
| 1a | 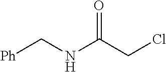 [SMILES: O=C(CCl)NCc1ccccc1] |
| 1b | 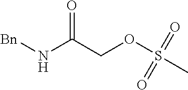 [SMILES: CS(=O)(=O)OCC(=O)NCc1ccccc1] |
| 1c | 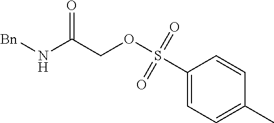 [SMILES: Cc1ccc(S(=O)(=O)OCC(=O)NCc2ccccc2)cc1] |
| 1d | 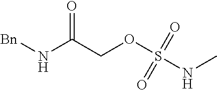 [SMILES: CNS(=O)(=O)OCC(=O)NCc1ccccc1] |
| 1e | 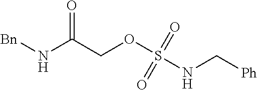 [SMILES: O=C(COS(=O)(=O)NCc1ccccc1)NCc1ccccc1] |
| 1f | 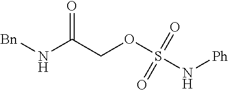 [SMILES: O=C(COS(=O)(=O)Nc1ccccc1)NCc1ccccc1] |
| 1g | 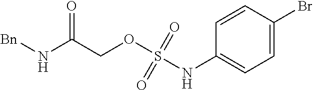 [SMILES: O=C(COS(=O)(=O)Nc1ccc(Br)cc1)NCc1ccccc1] |
| 1h | 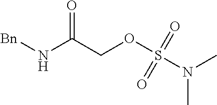 [SMILES: CN(C)S(=O)(=O)OCC(=O)NCc1ccccc1] |
| 1i | 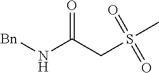 |
| 1j | 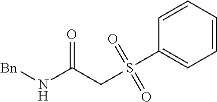 [SMILES: O=C(CS(=O)(=O)c1ccccc1)NCc1ccccc1] |
| 2a | 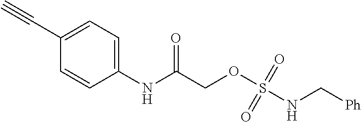 [SMILES: C#Cc1ccc(NC(=O)COS(=O)(=O)NCc2ccccc2)cc1] |
| 2b | 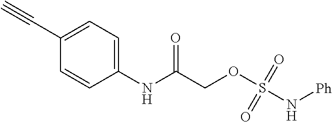 [SMILES: C#Cc1ccc(NC(=O)COS(=O)(=O)Nc2ccccc2)cc1] |
| 2c | 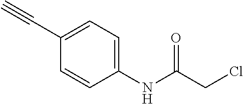 |

GSH consumption assays was conducted (5 mM GSH, 200 μM electrophile at pH 8, 14° C.; 4-nitro cyano-benzene was used as an internal standard) for all the sulfo compounds as well as benzyl acrylamide (BnA) and chloroacetamide (1a) (FIG. 5A). A sample from the reaction mixture was injected to an LC/MS every hour and the decrease in starting material was quantified over the course of the reaction. For example, the LCMS chromatogram (at 220 nm) of sulfamate 1d at t=0 h and at t=5 h shows an increase in GSH adduct and a decrease in starting material (FIG. 4A). The sulfonate esters, mesyl (1b) and tosyl (1c) groups showed similar reactivities to the chloroacetamide (1a) with a half-life of 50 min. On the other hand, methyl sulfamate (1d) and benzyl sulfamate (1e) showed an order of magnitude less reactivity than chloroacetamide (1a) with equal or lower reactivity to that of the unsubstituted acrylamide (BnA; FIG. 4B).

Surprisingly, phenyl sulfamate (if; t½=˜25 h) and 4-bromo phenyl sulfamate (1g; t½>100 h) exhibit much lower reactivity, possibly because the release of electrons from the amine to the sulfur increases conjugation and stabilization (FIG. 4E). Finally, dimethyl sulfamate (1 h) also did not react under these reaction conditions (t½>100 h). α-sulfone acetamides (1i and 1j) did not form a covalent bond with GSH under the assay conditions or even at temperatures up to 37° C. for 24 h (FIG. 6). The thiol reactivities of these model compounds were assessed using a DTNB reactivity assay (FIG. 4C), which showed similar results to the GSH consumption assay (FIG. 5B; FIG. 4D).

Sulfamate model compounds (1d-1j) are an order of magnitude less reactive to GSH than the corresponding chloroacetamide (1a) and sulfonate compounds (1b-1c). Whereas the aryl-substituted and secondary amine substituted sulfamates show even drastically lower reactivity (FIG. 5B). This may be because the electronics of the amine reduces the electrophilicity of the α-carbon of the sulfamate acetamides (FIG. 4E). The proteomic reactivity of the sulfamates reflected similar reactivity trends with the more reactive chloroacetamide (2c) and benzyl sulfamate acetamide (2a) enriching more proteins than the low reactivity phenyl sulfamate acetamide (2b; FIG. 5).

The fact that additional bands were detected with the phenyl sulfamate acetamide compared to the chloroacetamide may suggest that the extra recognition of the phenyl sulfamate mediates additional interactions with some protein targets. Similar to substituted methacrylamides, these compounds also leave identical adducts on proteomically labeled cysteines. Hence, mixtures of sulfamates can serve as probes for quantitative chemoproteomics with potentially increased coverage.

### Example 7

### Synthesis of Model Compounds

### Synthesis of N-benzyl-2-chloroacetamide (1a)

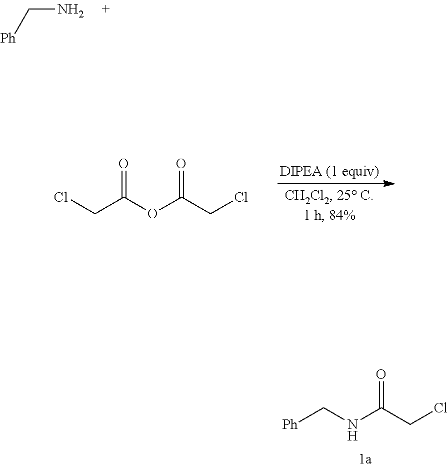 [SMILES: NCc1ccccc1 ; O=C(CCl)OC(=O)CCl ; O=C(CCl)NCc1ccccc1]

To a stirred solution of Bn-NH₂ (108 μL, 1 mmol) in anhydrous DCM (2 mL), DIPEA (178 μL, 1 mmol) and chloroacetic anhydride (170 mg, 1 mmol) were added at 25° C. The reaction mixture was stirred at room temperature for 1 h. After completion of the reaction (as monitored by LC-MS), CH₂Cl₂ was concentrated in vacuo and the crude product was purified using combi flash column chromatography using EtOAc:Hexane as eluent to give 1a in 154 mg (yield=84%).

¹H NMR (500 MHz, CDCl₃): δ 4.12 (s, 2H), 4.51 (d, J=5.8 Hz, 2H), 6.88 (br. s., 1H), 7.28-7.34 (m, 3H), 7.34-7.42 (m, 2H).

¹³C NMR (126 MHz, CDCl₃): δ 42.6, 43.9, 127.8, 127.8, 128.8, 137.2, 165.8.

ESI-MS (m/z): calculated for C₉Hn₁ClNO [M+H]+: 184.05; found: [M+H]+: 184.82.

### Synthesis of N-benzyl-2-hydroxyacetamide (Pre-1)

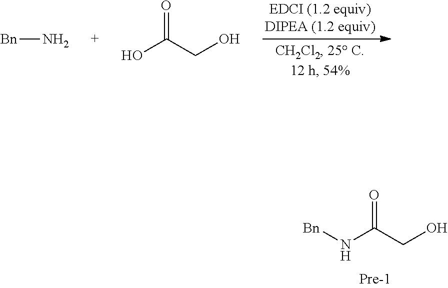 [SMILES: NCc1ccccc1 ; O=C(O)CO ; O=C(CO)NCc1ccccc1]

To a stirred solution of Bn-NH₂ (324 μL, 3 mmol) in anhydrous DCM (6 mL), 1-Ethyl-3-(3-dimethylaminopropyl)carbodiimide hydrochloride (690 mg, 3.6 mmol) and DIPEA (600 μL, 3.6 mmol) were added at 25° C. The reaction mixture was stirred at room temperature for 12 h. After completion of the reaction (as monitored by LC-MS), the reaction mixture was evaporated under vacuo and the crude product was purified using combi flash column chromatography with MeOH:EtOAc (2:8) as eluent to give Pre-1 was colorless solid in 267 mg (yield=54%).

ESI-MS (m/z): calculated for C₉H₁₂NO₂ [M+H]+: 166.08; found: [M+H]+: 166.30.

### Synthesis of 2-(benzylamino)-2-oxoethyl methanesulfonate (1b)

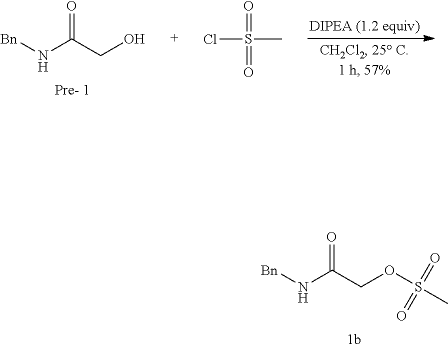

To a stirred solution of Pre-1 (16.5 mg, 0.1 mmol) in CH₂Cl₂ (1 mL), methane sulfonyl chloride (9.2 μL, 0.12 mmol, d=1.48), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1b in 13.8 mg (57% yield).

¹H NMR (400 MHz, CDCl₃) δ: 2.50 (br. s., 1H), 3.07 (s, 3H), 4.47 (d, J=5.7 Hz, 2H), 4.66 (s, 2H), 6.67 (br. s., 1H), 7.19-7.40 (m, 4H).

¹³C NMR (101 MHz, CDCl₃) δ 37.8, 43.4, 66.6, 127.8, 127.8, 128.8, 137.0, 165.2.

ESI-MS (m/z): calculated for C₁₀H₁₄NO₄S [M+H]+: 244.06; found: [M+H]+: 244.64.

### Synthesis of 2-(benzylamino)-2-oxoethyl 4-methylbenzenesulfonate (1c)

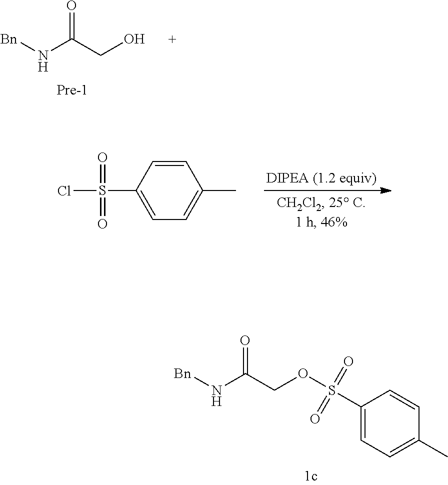 [SMILES: O=C(CO)NCc1ccccc1 ; Cc1ccc(S(=O)(=O)Cl)cc1 ; Cc1ccc(S(=O)(=O)OCC(=O)NCc2ccccc2)cc1]

To a stirred solution of Pre-1 (16.5 mg, 0.1 mmol) in CH₂Cl₂ (1 mL), 4-toluenesulfonyl chloride (22.8 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1c in 14.6 mg (46% yield).

¹H NMR (500 MHz, CDCl₃) δ: 2.47 (s, 3H), 4.46 (d, J=5.9 Hz, 2H), 4.49 (s, 2H), 6.66 (br. s., 1H), 7.25 (d, J=7.0 Hz, 2H), 7.28-7.33 (m, 1H), 7.37 (d, J=8.1 Hz, 2H), 7.34 (d, J=7.6 Hz, 2H), 7.79 (d, J=8.3 Hz, 2H).

¹³C NMR (126 MHz, CDCl₃) δ: 21.7, 43.3, 66.9, 127.8, 128.1, 128.8, 130.2, 131.6, 137.0, 145.9, 165.2.

ESI-MS (m/z): calculated for C₁₆H₁₈NO₄S [M+H]+: 320.10; found: [M+H]+: 320.55.

### Synthesis of 2-(benzylamino)-2-oxoethyl methylsulfamate (1d)

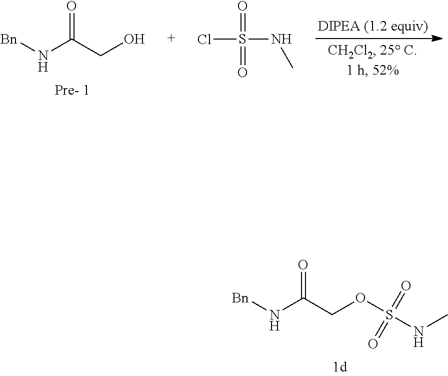

To a stirred solution of Pre-1 (16.5 mg, 0.1 mmol) in CH₂Cl₂ (1 mL), N-methylsulfamoyl chloride (15.4 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1d in 13.4 mg (52% yield).

¹H NMR (400 MHz, CDCl₃) δ: 2.79 (d, J=5.1 Hz, 3H), 4.50 (d, J=5.7 Hz, 2H), 4.59 (s, 2H), 5.24 (d, J=4.6 Hz, 1H), 6.79 (br. s., 1H), 7.18-7.45 (m, 4H)

¹³C NMR (100 MHz, CDCl₃) δ: 29.8, 43.3, 67.2, 127.8, 128.8, 137.0, 166.0.

ESI-MS (m/z): calculated for C₁₀H₁₅N₂O₄S [M+H]+: 259.08; found: [M+H]+: 259.64.

### Synthesis of 2-(benzylamino)-2-oxoethyl benzylsulfamate (1e)

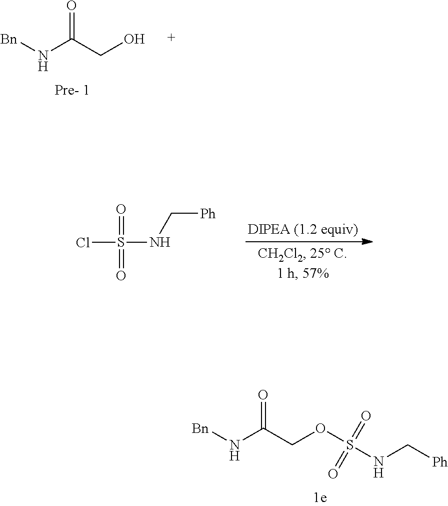 [SMILES: O=C(CO)NCc1ccccc1 ; O=S(=O)(Cl)NCc1ccccc1 ; O=C(COS(=O)(=O)NCc1ccccc1)NCc1ccccc1]

To a stirred solution of Pre-1 (16.5 mg, 0.1 mmol) in CH₂Cl₂ (1 mL), benzylsulfamoyl chloride (24 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1e in 19 mg (57% yield).

¹H NMR (500 MHz, CDCl₃) δ: 4.31 (d, J=5.6 Hz, 2H), 4.45 (s, 2H), 4.49 (s, 2H), 5.40 (t, J=5.4 Hz, 1H), 6.48 (br. s., 1H), 7.24-7.29 (m, 3H), 7.29-7.35 (m, 6H), 7.35-7.40 (m, 2H).

¹³C NMR (126 MHz, CDCl₃) δ: 43.3, 48.0, 67.1, 127.8, 127.8, 128.1, 128.5, 128.8, 129.0, 135.7, 137.0, 165.6.

ESI-MS (m/z): calculated for C₁₆H₁₉N₂O₄S [M+H]+: 335.11; found: [M+H]+: 335.18.

### Synthesis of 2-(benzylamino)-2-oxoethyl phenylsulfamate (1J)

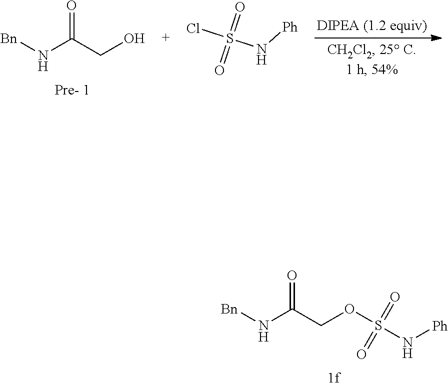 [SMILES: O=C(CO)NCc1ccccc1 ; O=S(=O)(Cl)Nc1ccccc1 ; O=C(COS(=O)(=O)Nc1ccccc1)NCc1ccccc1]

To a stirred solution of Pre-1 (16.5 mg, 0.1 mmol) in CH₂Cl₂ (1 mL), phenylsulfamoyl chloride (22.5 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid if in 17.2 mg (54% yield).

¹H NMR (400 MHz, CDCl₃) δ: 4.42 (d, J=5.7 Hz, 2H), 4.66 (s, 2H), 6.55 (br. s., 1H), 7.21 (d, J=7.5 Hz, 4H), 7.33 (d, J=7.7 Hz, 4H), 7.49 (s, 1H).

¹³C NMR (126 MHz, CDCl₃) δ: 29.3, 42.7, 66.6, 119.7, 121.0, 124.7, 126.8, 127.3, 128.3, 129.1, 135.9, 162.4 (s).

ESI-MS (m/z): calculated for C₁₅H₁₇N₂O₄S [M+H]+: 321.09; found: [M+H]+: 321.57.

### Synthesis of 2-(benzylamino)-2-oxoethyl (4-bromophenyl)sulfamate (1g)

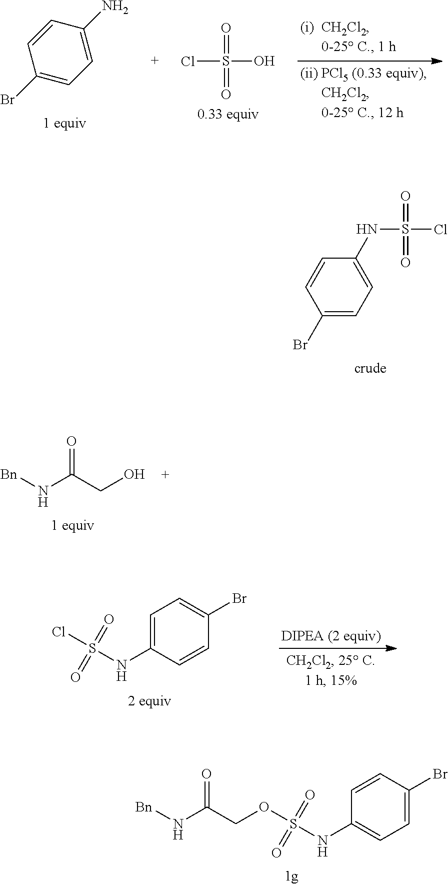 [SMILES: Nc1ccc(Br)cc1 ; O=S(=O)(O)Cl ; O=S(=O)(Cl)Nc1ccc(Br)cc1 ; O=C(CO)NCc1ccccc1 ; O=S(=O)(Cl)Nc1ccc(Br)cc1 ; O=C(COS(=O)(=O)Nc1ccc(Br)cc1)NCc1ccccc1]

To a stirred solution of 4-bromo aniline (172 mg, 1 mmol) in CH₂Cl₂ (1 mL), chloro methane sulfonyl chloride (22 μL, 0.33 mmol,) was added at 0° C. The reaction mixture was stirred at room temperature for 1 h. After completion of the reaction organic layer was concentrated in vacuo. The crude product was dissolved CH₂Cl₂ (1 mL) and PCl₅ (68 mg, 0.33 mmol) was added at 0° C. The reaction mixture was stirred at room temperature for 12 h. After completion of the reaction (as monitored by LC-MS), the reaction mixture is filtered and washed with dichloromethane. The filtrate was concentrated and used as such for the next reaction.

To a stirred solution of Pre-1 (16.5 mg, 0.1 mmol) in CH₂Cl₂ (1 mL), 4-bromo phenylsulfamoyl chloride (0.2 mmol), and DIPEA (35 μL, 0.2 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 1g in 6 mg (15% yield).

¹H NMR (500 MHz, CDCl₃) δ: 4.45 (d, J=5.8 Hz, 2H), 4.66 (s, 2H), 6.36-6.49 (m, 1H), 7.04-7.11 (m, 2H), 7.17 (s, 1H), 7.23 (d, J=6.9 Hz, 2H), 7.32-7.39 (m, 3H), 7.43-7.50 (m, 2H).

¹³C NMR (126 MHz, CDCl₃) δ: 43.7, 68.3, 114.6, 122.8, 128.1, 128.2, 129.2, 133.1, 137.6, 165.8, 173.3.

ESI-MS (m/z): calculated for C₁₅H₁₆Br⁸¹N₂O₄S [M+H]+: 401.00; found: [M+H]+: 401.22. C₁₅H₁₆Br⁹N₂O₄S [M+H]+: 399.00; found: [M+H]+: 399.15.

### Synthesis of 2-(benzylamino)-2-oxoethyl dimethylsulfamate (1 h)

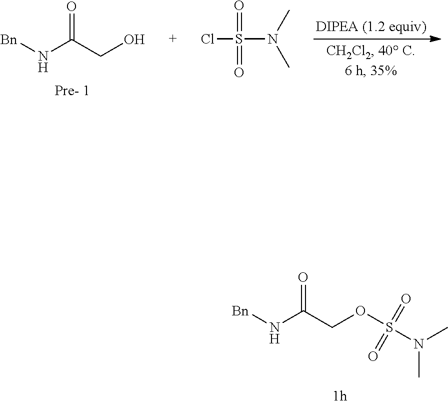

To a stirred solution of Pre-1 (16.5 mg, 0.1 mmol) in CH₂Cl₂ (1 mL), N,N-dimethylsulfamoyl chloride (16.9 mg, 0.12 mmol), and DIPEA (20.4 μL, 0.12 mmol) were added at 40° C. The reaction mixture was stirred at room temperature for 6 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1 h in 9.5 mg (35% yield).

¹H NMR (500 MHz, CD₃OD) δ: 2.92 (s, 6H), 4.46 (s, 2H), 4.63 (s, 2H), 7.23-7.31 (m, 1H), 7.33 (s, 4H).

¹³C NMR (125 MHz, CD₃OD) δ: 38.8, 44.0, 48.6, 68.3, 128.5, 128.8, 129.7, 139.7, and 168.6.

ESI-MS (m/z): calculated for C₁₁H₁₇N₂O₄S [M+H]+: 273.09; found: [M+H]+: 273.86.

### Synthesis of N-benzyl-2-(methylsulfonyl)acetamide (1i)

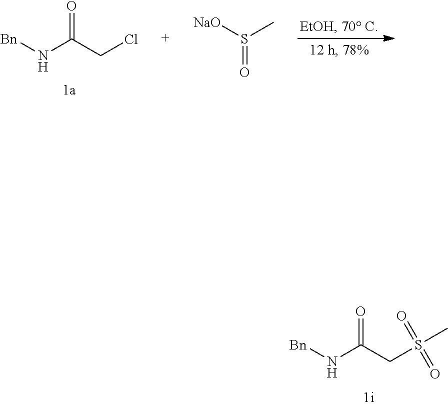

To a stirred solution of 1a (18.3 mg, 0.1 mmol) in ethanol (1 mL), sodium methanesulfinate (20.4 mg, 0.2 mmol) was added at 25° C. The reaction mixture was stirred at room temperature for 12 h at 70° C. After completion of the reaction (as monitored by LC-MS), ethanol was concentrated in vacuo and the crude product was purified by preparative HPLC using water:ACN (0.1% formic acid) solvent gradient to afford white solid 1i in 17.7 mg (78% yield).

ESI-MS (m/z): calculated for C₁₀H₁₄NO₃S [M+H]+: 228.07; found: [M+H]+: 228.22.

### Synthesis of N-benzyl-2-(phenylsulfonyl)acetamide (1j)

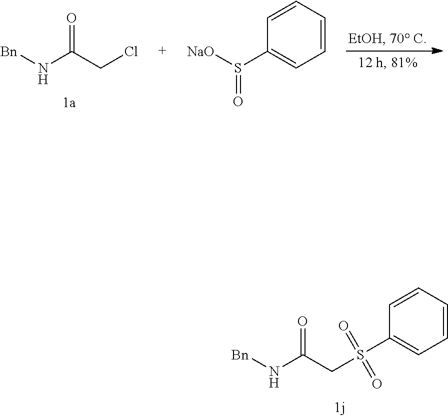

To a stirred solution of 1a (23 mg, 0.1 mmol) in ethanol (1 mL), sodium phenylsulfinate (20.2 mg, 0.2 mmol) was added at 25° C. The reaction mixture was stirred at room temperature for 12 h at 70° C. After completion of the reaction (as monitored by LC-MS), ethanol was concentrated in vacuo and the crude product was purified by preparative HPLC using water:ACN (0.1% formic acid) solvent gradient to afford white solid 1j in 23.4 mg (81% yield).

ESI-MS (m/z): calculated for C₁₅H₁₆NO₃S [M+H]+: 290.09; found: [M+H]+: 290.65.

### Synthesis of N-benzyl-2-hydroxyacetamide (Pre-2)

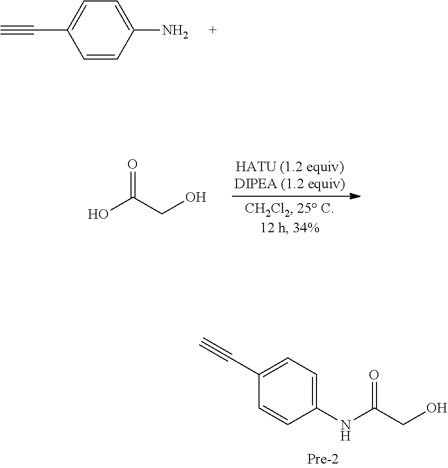

To a stirred solution of 4-ethynylaniline (117 mg, 1 mmol) in anhydrous DCM (6 mL), HATU (456 mg, 1.2 mmol) and DIPEA (200 μL, 1.2 mmol) were added at 25° C. The reaction mixture was stirred at room temperature for 12 h. After completion of the reaction (as monitored by LC-MS), the reaction mixture was evaporated under vacuo and the crude product was purified using combi flash column chromatography with MeOH:EtOAc (2:8) as eluent to give Pre-2 was colorless solid in 59 mg (yield=34%).

¹H NMR (500 MHz, CDCl₃): δ: 3.00 (s, 1H), 3.24 (br. s., 1H), 4.02 (s, 3H), 7.35 (d, J=8.0 Hz, 2H), 7.46 (d, J=8.1 Hz, 2H).

¹³C NMR (126 MHz, CDCl₃) δ: 65.8, 80.8, 87.2, 121.9, 123.4, 136.8, 141.5, 175.3 (s).

ESI-MS (m/z): calculated for C₁₀H₉NO₂S [M+H]+: 176.07; found: [M+H]+: 176.09.

### Synthesis of 2-((4-ethynylphenyl)amino)-2-oxoethyl benzylsulfamate (2a)

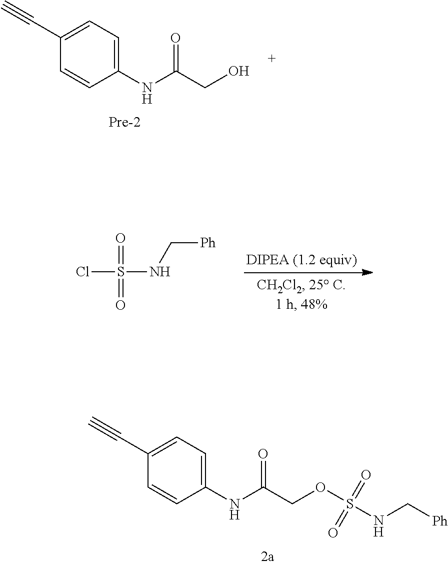 [SMILES: C#Cc1ccc(NC(=O)CO)cc1 ; O=S(=O)(Cl)NCc1ccccc1 ; C#Cc1ccc(NC(=O)COS(=O)(=O)NCc2ccccc2)cc1]

To a stirred solution of Pre-2 (17.5 mg, 0.1 mmol) in CH₂Cl₂ (1 mL), benzylsulfamoyl chloride (24 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 2a in 16.5 mg (48% yield).

¹H NMR (400 MHz, CDCl₃) δ: 3.08 (s, 1H), 4.38 (d, J=5.7 Hz, 2H), 4.54-4.57 (m, 2H), 5.17 (t, J=5.5 Hz, 1H), 7.33-7.41 (m, 4H), 7.42-7.53 (m, 4H), 7.80 (br. s., 1H).

¹³C NMR (100 MHz, CDCl₃) δ: 48.4, 67.6, 83.4, 120.2, 128.4, 129.0, 129.4, 133.3, 135.7, 137.1, 163.7.

ESI-MS (m/z): calculated for C₁₇H₁₇N₂O₄S [M+H]+: 345.09; found: [M+H]+: 345.67.

### Synthesis of 2-((4-ethynylphenyl)amino)-2-oxoethyl phenylsulfamate (2b)

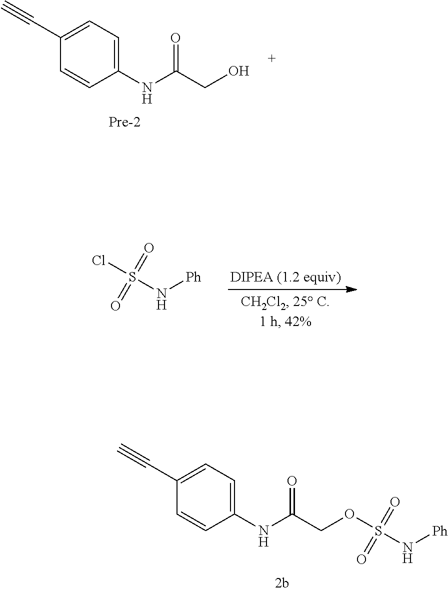 [SMILES: C#Cc1ccc(NC(=O)CO)cc1 ; O=S(=O)(Cl)Nc1ccccc1 ; C#Cc1ccc(NC(=O)COS(=O)(=O)Nc2ccccc2)cc1]

To a stirred solution of Pre-2 (17.5 mg, 0.1 mmol) in CH₂Cl₂ (1 mL), phenylsulfamoyl chloride (22.5 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 2b in 13.8 mg (42% yield).

¹H NMR (500 MHz, CDCl₃) δ: 3.07 (s, 1H), 4.74 (s, 2H), 7.26 (br. s., 2H), 7.28 (br. s., 2H), 7.37-7.42 (m, 3H), 7.44-7.47 (m, 2H).

¹³C NMR (126 MHz, CDCl₃) δ: 67.9, 68.6, 83.0, 119.8, 121.2, 126.5, 129.9, 133.0, 137.3, 141.0, 163.2.

ESI-MS (m/z): calculated for C₁₆H₁₅N₂O₄S [M+H]+: 331.08; found: [M+H]+: 331.97.

### Synthesis of 2-chloro-N-(4-ethynylphenyl)acetamide (2c)

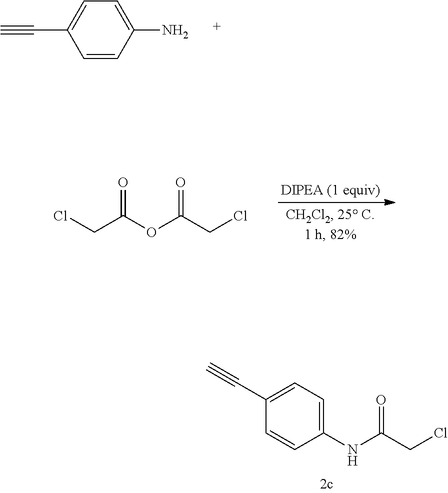

To a stirred solution of 4-ethynylaniline (11.7 mg, 1 mmol) in anhydrous DCM (0.5 mL), DIPEA (17.8 μL, 1 mmol) and chloroacetic anhydride (17 mg, 1 mmol) were added at 25° C. The reaction mixture was stirred at room temperature for 1 h. After completion of the reaction (as monitored by LC-MS), CH₂Cl₂ was concentrated in vacuo and the crude compound was purified by preparative HPLC using water:ACN (0.1% formic acid) solvent gradient to afford white solid 2c in 15.8 mg (yield=82%).

¹H NMR (400 MHz, CDCl₃) δ: 3.08 (s, 1H), 4.20 (s, 2H), 7.41-7.60 (m, 4H), 8.28 (br. s., 1H).

¹³C NMR (100 MHz, CDCl₃) δ: 42.8, 83.0, 118.8, 119.6, 133.0, 137.0, 163.8.

ESI-MS (m/z): calculated for C₁₀H₉ClNO [M+H]+: 194.04; found: [M+H]+: 194.82.

### Example 8

The SARS-CoV-2 main protease (Mpro; or 3CL-protease) is an attractive target for antiviral development due to its essential role in viral replication, a large degree of conservation across coronaviruses, and dissimilarity of its structure and substrate profile to human proteases. Studies during and after the 2003 SARS pandemic established the linkage between Mpro inhibition and antiviral activity in cell culture. This is corroborated by recent in vitro and in vivo studies for SARS-CoV-2 and the recent clinical success of nirmatrelvir (the Mpro inhibitor component of Paxlovid) and ensitrevir (Xocova).

Mpro contains a catalytic Cysteine (C145). Chloroacetamide covalent binders for Mpro (compounds A, B, C; Table 2) were reported previously. This Example provides sulfamate analogs of these chloroacetamide inhibitors which were synthesized and tested to see if the potency against Mpro is improved or maintained.

While all sulfamate analogs retained some activity (Tables 3-5), analogs of compound B were ˜2 fold less active in the biochemical assay (Table 4), and analogs of compound C were significantly less active than their chloroacetamide counterpart (Table 5). For compound A however, we were able to significantly improve potency by switching from a chloroacetamide to a sulfamate electrophile. For example A6 is about 2-fold more potent and A7, A8 are about 7-fold more active than that parent A.

TABLE 2
Original chloroacetamide Mpro inhibitors

| Name | Structure | IC50 (μM) |
|---|---|---|
| A | 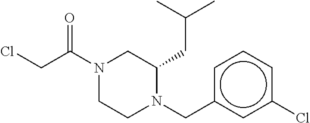 | 0.700 ± 0.0837 (n = 2) |
| B | 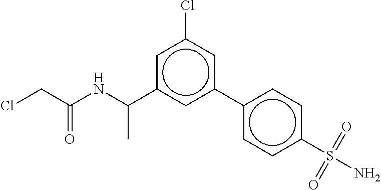 | 0.466 |
| C | 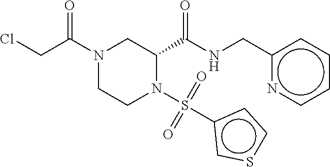 | 0.307 |

TABLE 3
Sulfamate analogs of compound A.

| Name | Structure | IC50 (μM) |
|---|---|---|
| A1 | 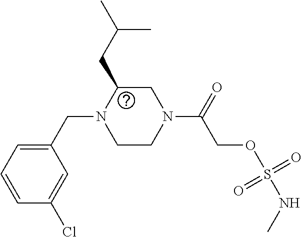 [SMILES: CNS(=O)(=O)OCC(=O)N1CCN(Cc2cccc(Cl)c2)[C@@H](CC(C)C)C1] | 1.67 |
| A2 | 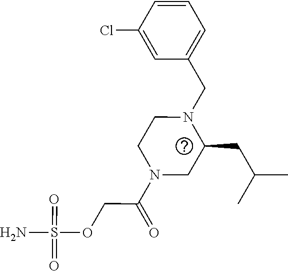 [SMILES: CC(C)C[C@H]1CN(C(=O)COS(N)(=O)=O)CCN1Cc1cccc(Cl)c1] | 6.69 |
| A3 | 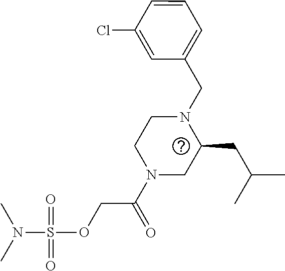 [SMILES: CC(C)C[C@H]1CN(C(=O)COS(=O)(=O)N(C)C)CCN1Cc1cccc(Cl)c1] | 1.029 |
| A4 | 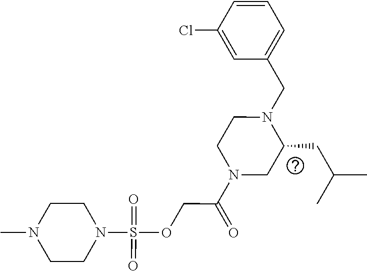 [SMILES: CC(C)C[C@@H]1CN(C(=O)COS(=O)(=O)N2CCN(C)CC2)CCN1Cc1cccc(Cl)c1] | >99.500 |
| A5 | 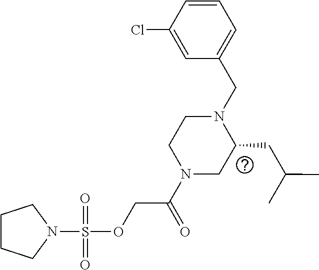 [SMILES: CC(C)C[C@@H]1CN(C(=O)COS(=O)(=O)N2CCCC2)CCN1Cc1cccc(Cl)c1] | 1.096 1.484 |
| A6 | 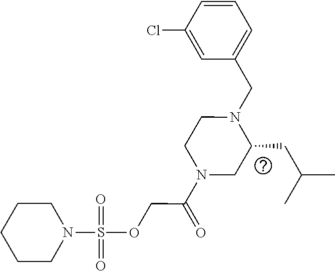 [SMILES: CC(C)C[C@@H]1CN(C(=O)COS(=O)(=O)N2CCCCC2)CCN1Cc1cccc(Cl)c1] | 0.316 0.392 |
| A7 | 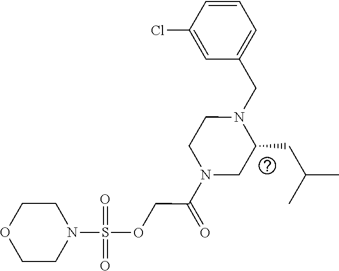 [SMILES: CC(C)C[C@@H]1CN(C(=O)COS(=O)(=O)N2CCOCC2)CCN1Cc1cccc(Cl)c1] | 0.063 0.120 |
| A8 | 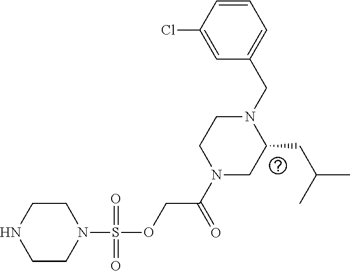 [SMILES: CC(C)C[C@@H]1CN(C(=O)COS(=O)(=O)N2CCNCC2)CCN1Cc1cccc(Cl)c1] | 0.120 |
| A9 | 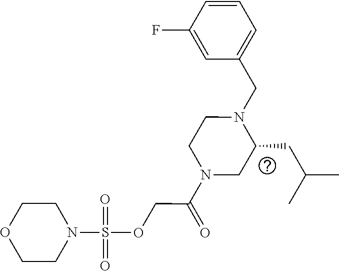 [SMILES: CC(C)C[C@@H]1CN(C(=O)COS(=O)(=O)N2CCOCC2)CCN1Cc1cccc(F)c1] | 0.088* 0.445 1.258 |
| A10 | 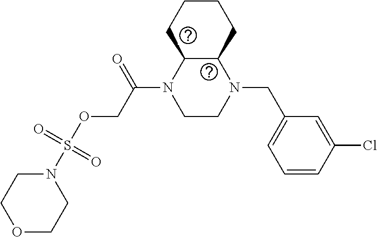 [SMILES: O=C(COS(=O)(=O)N1CCOCC1)N1CCN(Cc2cccc(Cl)c2)[C@@H]2CCCC[C@@H]21] | 0.096* 0.494 5.88 |
| A11 | 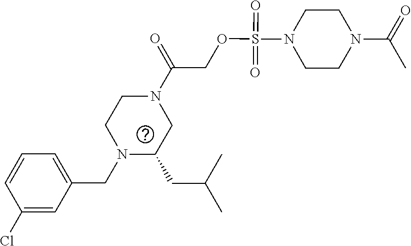 [SMILES: CC(=O)N1CCN(S(=O)(=O)OCC(=O)N2CCN(Cc3cccc(Cl)c3)[C@@H](CC(C)C)C2)CC1] | 0.072* 0.477 1.258 |

*Compounds were measured on various days and we saw a right shift in potency which we attribute to DMSO instability, the lowest value is the most representative
**Compounds were all tested as racemates and not enantiopure isomers.

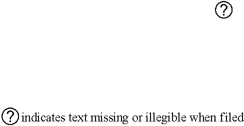

TABLE 4
Sulfamate analogs of compound B.

| | | IC50 |
|---|---|---|
| Name | Structure | (μM) |
| B1 | 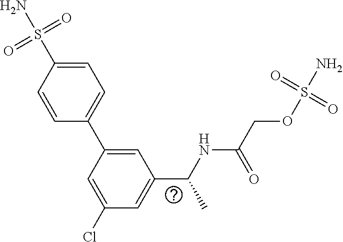 [SMILES: C[C@@H](NC(=O)COS(N)(=O)=O)c1cc(Cl)cc(-c2ccc(S(N)(=O)=O)cc2)c1] | 1.771 |
| B2 | 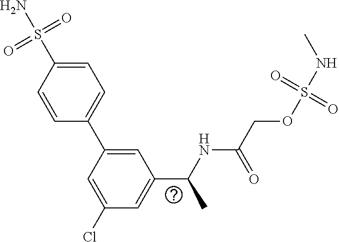 [SMILES: CNS(=O)(=O)OCC(=O)N[C@@H](C)c1cc(Cl)cc(-c2ccc(S(N)(=O)=O)cc2)c1] | 1.732 |

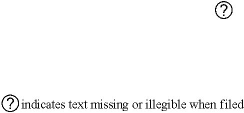

TABLE 5
Sulfamate analogs of compound C.

| Name | Structure | IC50 (μM) |
|---|---|---|
| C1 | 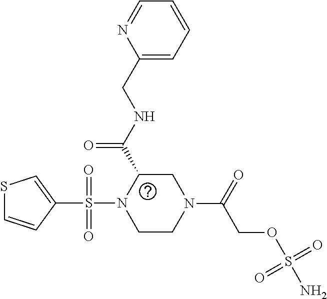 [SMILES: NS(=O)(=O)OCC(=O)N1CCN(S(=O)(=O)c2ccsc2)[C@H](C(=O)NCc2ccccn2)C1] | 80.399 |
| C2 | 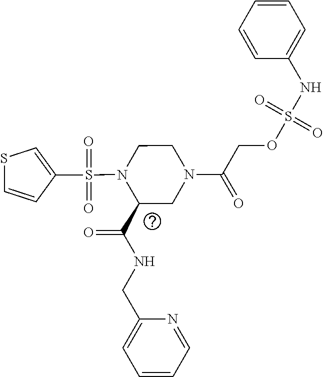 [SMILES: O=C(NCc1ccccn1)[C@@H]1CN(C(=O)COS(=O)(=O)Nc2ccccc2)CCN1S(=O)(=O)c1ccsc1] | 17.59 |
| C3 | 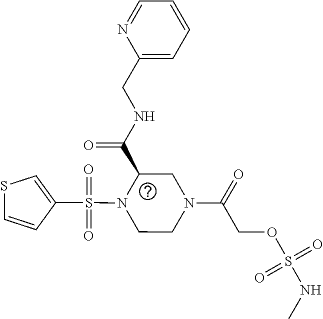 [SMILES: CNS(=O)(=O)OCC(=O)N1CCN(S(=O)(=O)c2ccsc2)[C@@H](C(=O)NCc2ccccn2)C1] | 16.302 |

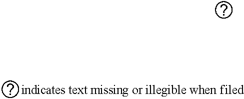

While certain features and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure herein.

1. A Covalent Ligand Directed Releasing (CoLDR) compound or pharmaceutically acceptable salt thereof, wherein the compound is represented by the structure of formula I: 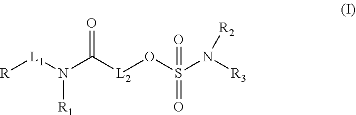
wherein:
R is a protein binding ligand, wherein the nitrogen (NR₁) is linked to the protein binding ligand via L₁ linker or L₁ is a bond and the nitrogen is an atom within the protein binding ligand;
R₁ and R₂ are each independently H, substituted or unsubstituted: linear or branched alkyl, linear or branched alkenyl, linear or branched alkynyl, aryl, alkylaryl, cycloalkyl, heterocycloalkyl, heteroaryl;
R₃ is H, substituted or unsubstituted: linear or branched alkyl, linear or branched alkenyl, linear or branched alkynyl, aryl, alkyl aryl, cycloalkyl, heterocycloalkyl, heteroaryl a fluorescent probe, a chemiluminescent probe or a radiolabeled probe or a bioactive group;
L₁ is a bond, or a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl or an ether group; and
L₂ is a bond, a linker comprising substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl or an ether group.

2. The CoLDR compound according to claim 1, wherein the protein binding ligand is ibrutinib and the compound is represented by the structure of formula II:

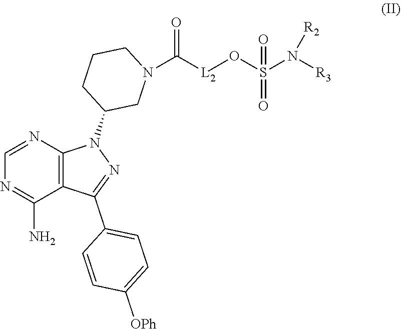

3. The CoLDR compound according to claim 2, wherein the compound is represented by the structure of formula III:

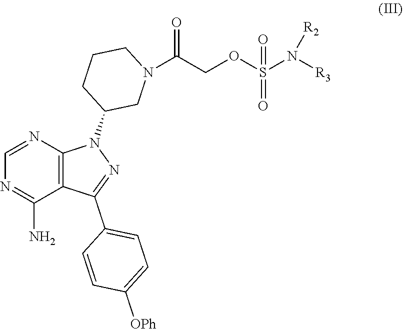

4. The CoLDR compound according to claim 1 wherein R₂ is H.

5. The CoLDR compound according claim 1, wherein L₂ is methylene (—CH₂—).

6. The CoLDR compound according to claim 1, wherein the compounds are selected from:

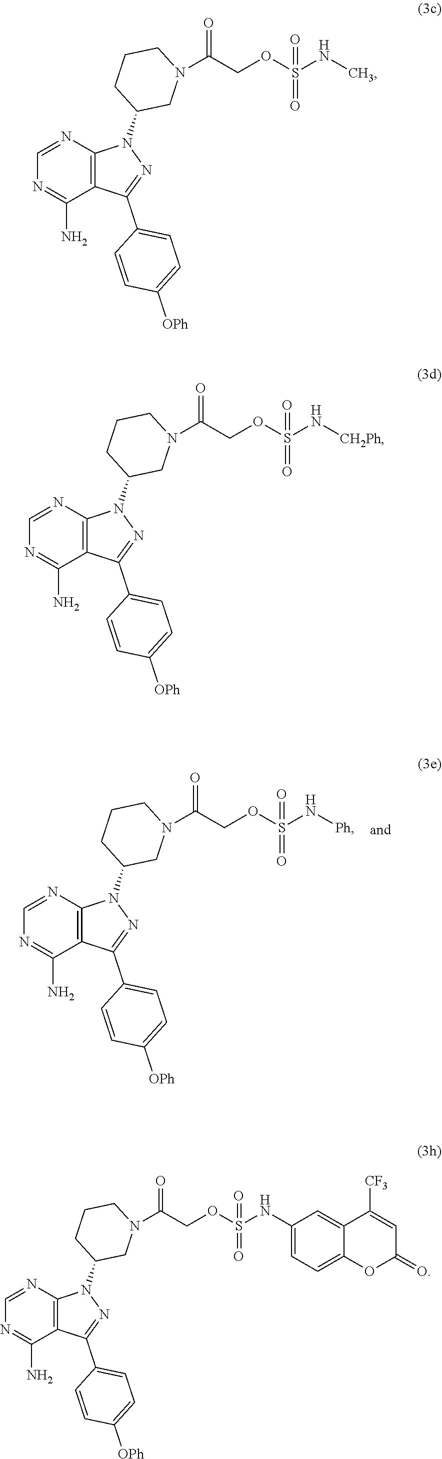

7. The CoLDR compound according to claim 1 wherein upon interaction between a protein and the protein binding ligand, a functionalized amine [NH(R₂)(R₃)] and sulfur trioxide are released.

8. The CoLDR compound according to claim 1, wherein a covalent bond is formed between a protein and the protein binding ligand.

9. The CoLDR compound according to claim 8, wherein the covalent bond is formed via a nucleophilic moiety of the protein being a thiol, an amine or a hydroxyl group and the alpha sulfamate acetamide of the compounds of formula I, II or III.

10. A pharmaceutical composition comprising the compounds according to claim 1 and a pharmaceutical acceptable carrier.

11. A protein sensor or a protein label comprising a Covalent Ligand Directed Releasing (CoLDR) compound according to claim 1, wherein R3 is a fluorescent probe or a chemiluminescent probe, wherein, upon interaction between a protein and the protein binding ligand, the fluorescent probe or the chemiluminescent probe is released and the protein binding ligand is covalently attached to the protein and thereby results in change in fluorescence or chemiluminescence of the probes.

12. The protein sensor according to claim 11, wherein a covalent bond is formed between the protein and the protein binding ligand.

13. The protein sensor according to claim 12, wherein the covalent bond is formed via a nucleophilic group of the protein being a thiol, an amine or a hydroxyl group and the alpha sulfamate acetamide of the CoLDR compound of formula I, II or III.

14. The CoLDR compound according claim 2, wherein L₂ is methylene (—CH₂—).
